(19) United States
(12) Patent Application Publication
(10) Pub. No.: US 2026/0308125 A1
(43) Pub. Date: Oct. 8, 2026

(54) HETEROCYCLIC CARBOXYLIC ACID HYDRAZIDE LIGAND AND USE THEREOF IN COPPER-CATALYZED ARYL HALIDE COUPLING REACTION

(71) Applicant: SHANGHAI INSTITUTE OF ORGANIC CHEMISTRY, CHINESE ACADEMY OF SCIENCES, Shanghai (CN)

(72) Inventors: Dawei MA, Shanghai (CN); Lanting XU, Shanghai (CN); Sailuo LI, Shanghai (CN); Hua SHAO, Shanghai (CN); Xiang LI, Shanghai (CN)

(21) Appl. No.: 19/478,998

(22) PCT Filed: Apr. 17, 2024
(86) PCT No.: PCT/CN2024/088310

§ 371 Date: Oct. 27, 2025

(30) Foreign Application Priority Data
Apr. 25, 2023 (CN) 202310455450.9

(51) Int. Cl. C07D 213/81; C07B 43/04; C07D 401/12; C07D 401/14; C07D 403/12; C07D 405/12; C07D 417/12; C07D 471/04; C07D 491/048

(52) U.S. Cl. CPC C07D 213/81; C07B 43/04; C07D 401/12; C07D 401/14; C07D 403/12; C07D 405/12; C07D 417/12; C07D 471/04; C07D 491/048

(57) ABSTRACT

A heterocyclic carboxylic acid hydrazide ligand and the use thereof in copper-catalyzed aryl halide coupling reaction are provided. Specifically, the compound of formula 1 can be used as a ligand in the copper-catalyzed aryl halide coupling reaction, and is used for catalyzing a coupling reaction for forming aryl halides such as C—N, C—O, C—S and C—C bonds.

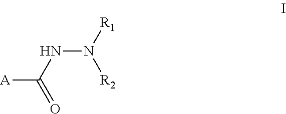

### TECHNICAL FIELD

The present invention relates to the field of organic synthesis. Specifically, the present invention provides a copper-catalyzed aryl halide coupling reaction promoted by heterocyclic carboxylic acid hydrazide ligands, particularly the reaction of coupling to generate C—N, C—O, C—S, C—C bonds.

### BACKGROUND

Transition metal-catalyzed cross-coupling reactions are one of the most fundamental methods in organic synthesis chemistry and the creation of functional molecules. By performing corresponding coupling reaction of aryl halides with suitable nucleophiles, such as organic amines, active methylene compounds, alkynes, phenols, alcohols, water, thiophenols, sodium sulfinates, sodium sulfides, and phosphate esters, under the action of a transition metal, a series of C—N, C—C, C—O, C—S, and C—P bonds can be efficiently constructed, which are widely used in fields such as medicine, pesticides, fine chemicals, and the synthesis of new materials.

In recent years, ligand-promoted copper-catalyzed Ullmann coupling reactions have achieved very significant development, forming an excellent complement to the classic palladium- and nickel-catalyzed coupling reactions. (Beletskaya, I. P.; Cheprakov, A. V. Organometallics 2012, 31, 7753). In comparison, copper catalysts are inexpensive, chemically stable, and have better biocompatibility, aligning with the needs of sustainable development and green chemistry, and thus possess greater potential for large-scale industrial applications. The development history of copper-catalyzed coupling reactions can be roughly divided into three stages: (1) traditional Ullmann coupling reaction. Early copper catalyzed cross coupling reactions required harsh reaction conditions and large amounts of catalyst (the temperature was typically above 200° C., most reactions required stoichiometric or sub-stoichiometric copper), leading to issues such as poor functional group tolerance, limited substrate applicability, numerous byproducts, and low yields. (2) The discovery and use of first generation bidentate ligands, including amino acids, phenanthrolines, diamines, diketones, Schiff bases, and other types of ligands. The addition of this first generation of ligands effectively improved the catalytic activity and selectivity of the reaction, avoided the use of stoichiometric copper catalysts, lowered the temperatures required for the reaction, and expanded the applicability of substrates, heralding a new era for copper-catalyzed coupling reactions. However, the first generation ligands and copper catalysts are generally used in high amounts (5-20 mol %), and the substrates are more concentrated in bromine/iodoaromatic hydrocarbons. For coupling reactions involving chlorinated compounds with smaller molecular weights and cheaper prices, there are almost no relevant reports except for a few special substrates (Docampo, M. L.; Pell ó n, R. F.); Estevez-Braun, A.; Ravelo, A. G. Eur. J. Org. Chem. 2007, 4111; Xia, N.; Taillefer, M. Chem. Eur. J. 2008, 14, 6037.). (3) The discovery and use of second-generation oxalyldiamide-type ligands. These ligands, for the first time, enabled the copper-catalyzed coupling of inexpensive and readily available aryl chlorides under mild conditions and reduced the catalyst dosages for coupling reactions of aryl bromides/iodides by two to three orders of magnitude. (Bhunia, S.; Pawar, G. G.; Kumar, S. V.; Jiang, Y.; Ma, D. Angew. Chem. Int. Ed. 2017, 56, 16136; Yang, Q.; Zhao, Y.; Ma, D. Org. Process Res. Dev. 2022, 26, 1690-1750.). Although oxalyldiamide-type ligands achieved a breakthrough in solving the problem of aryl chloride coupling, copper-catalyzed Ullmann coupling reactions still face challenges such as relatively high reaction temperatures, limited substrate applicability, and catalyst dosage that are too large to be comparable to palladium catalysts (the reaction temperature for chlorinated substrates is generally 120° C., and the catalyst dosage is generally 5-10 mol %, the reaction temperature for brominated substrates is generally around 80° C.). Copper catalyzed coupling reactions at room temperature remain rare (Kim, S.-T.; Strauss, M. J.; Cabr é, A.; Buchwald, S. L. J. Am. Chem. Soc. 2023, 145, 6966).

In summary, after a century of development and a path of revival, the copper-catalyzed Ullmann coupling reaction has become one of the most fundamental and important organic synthesis reactions for forming bonds between carbon atoms and nitrogen, oxygen, carbon, sulfur, and other atoms. (Dai Lixin. Ullmann Reaction Centenary and Recent Revival—Formation of Carbon Heteroatomic Bonds [J]. Advances in Chemistry, 2018, 30, 1257.). However, the activation of aryl chlorides/bromides under mild conditions, the achievement of ultra-low ligand and catalyst usages, and practical industrial applications remain significant challenges in this field. Developing highly efficient copper-catalyzed Ullmann coupling reactions to play a greater role in the creation of pharmaceutical and material molecules holds important academic research value and broad practical application prospects. The fundamental core to solving this problem lies in the development of more efficient and highly reactive ligands.

Overall, there is still a lack of a catalytic system for copper-catalyzed coupling reactions in the art that is widely applicable, industrially competitive, operates under mild conditions, uses low catalyst dosages, and is more environmentally friendly.

### SUMMARY OF THE PRESENT INVENTION

The purpose of the present invention is to provide a catalytic system that can be used for copper-catalyzed coupling reactions of aryl halides, in particular aryl chlorides under mild conditions, and aryl bromides under ultra-low catalyst dosage conditions.

In the first aspect of the present invention, provided is a use of a compound as shown in formula I, and an isomer thereof:

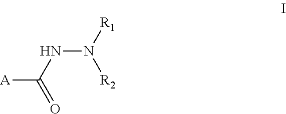

wherein, A is a saturated or unsaturated C5-C6 carbocyclyl, or a saturated or unsaturated 5- or 6-membered carbon heterocarbocyclyl, or a benzo saturated or unsaturated 5- or 6-membered carbon heterocarbocyclyl, wherein the carbon heterocarbocyclyl has 1 to 5 heteroatoms selected from the group consisting of: N, O, and S; A is optionally substituted by substituents selected from the group consisting of: hydroxyl, nitro, —NRaRb, halogen, C1-4 alkyl, C1-4 alkoxy, phenyl, and -oxo (i.e., two hydrogen atoms on the same carbon atom on the group are replaced by ═O);
Ra and Rb are each independently selected from the group consisting of: H, hydroxyl, amino, halogen, and C1-4 alkyl;
R₁ and R₂ are each independently selected from the group consisting of: H, substituted or unsubstituted C1-C6 alkyl, substituted or unsubstituted C1-C6 alkoxy, substituted or unsubstituted C3-C6 cycloalkyl, substituted or unsubstituted 3-6 membered heterocyclyl, substituted or unsubstituted C6-C20 aryl, substituted or unsubstituted 5-20 membered heteroaryl, substituted or unsubstituted benzyl; or, R₁ and R₂, together with the N atom to which they attached, form a 3-20 membered saturated or unsaturated carbocycle or heterocycle (such as substituted or unsubstituted pyrrole, indole, azoles, benzazole, and aromatic heterocycle);
as a ligand for copper-catalyzed coupling reactions of aryl halide; wherein, the aryl halide is selected from the group consisting of: aryl chloride, aryl bromide, aryl iodide, or a combination thereof;
unless otherwise specified, the “substituted” refers to one or more hydrogen atoms on the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl (alkyl-CO—), sulfonate, or two hydrogen atoms on two adjacent carbon atoms on the group are replaced by —(CH₂)n-(n is 1, 2, 3, 4, 5 or 6).

In another preferred embodiment, R₁ and R₂ are each independently selected from the group consisting of: H, substituted or unsubstituted C1-C6 alkyl, substituted or unsubstituted C1-C6 alkoxy, substituted or unsubstituted C6-C10 aryl, substituted or unsubstituted 5-10 membered heteroaryl, substituted or unsubstituted benzyl; or, R₁ and R₂, together with the N atom to which they attached, form a 3-20 membered substituted or unsubstituted saturated or unsaturated carbocycle or heterocycle.

In another preferred embodiment, R₁ and R₂ are each independently selected from the group consisting of: H, substituted or unsubstituted C1-C6 alkyl, substituted or unsubstituted phenyl, substituted or unsubstituted benzyl; or, R₁ and R₂, together with the N atom to which they attached, form a 3-20-membered carbocycle or heterocycle selected from the group consisting of: substituted or unsubstituted pyrrole, substituted or unsubstituted indole, substituted or unsubstituted azoles, substituted or unsubstituted benzazole, and substituted or unsubstituted aromatic heterocycle.

In another preferred embodiment, A is selected from the group consisting of: substituted or unsubstituted indolyl, substituted or unsubstituted benzimidazolyl, substituted or unsubstituted imidazolyl, substituted or unsubstituted thiazolyl, substituted or unsubstituted benzothiazolyl, substituted or unsubstituted thienyl, substituted or unsubstituted benzothienyl, substituted or unsubstituted furyl, substituted or unsubstituted benzofuranyl, substituted or unsubstituted saturated or unsaturated 5- to 6-membered heterocyclyl containing 1 or 2 N atoms (such as pyridyl, pyrazinyl, pyrimidinyl, pyridazinyl, azolyl, tetrahydropyrrolyl, hexahydropiperidinyl), substituted or unsubstituted phenyl, substituted or unsubstituted naphthyl, substituted or unsubstituted benzyl, substituted or unsubstituted quinolinyl, and substituted or unsubstituted isoquinolinyl;
the “substituted” refers to one or more hydrogen atoms being substituted with substituents selected from the group consisting of: hydroxyl, nitro, —NRaRb, halogen, C1-4 alkyl, C1-4 alkoxy, phenyl, and oxo (i.e., two hydrogen atoms on the same carbon atom in the group are replaced by ═O), wherein, Ra and Rb are each independently selected from the group consisting of: H, hydroxyl, amino, halogen, and C1-4 alkyl.

In another preferred embodiment, the compound has a structure shown in the following formula II:

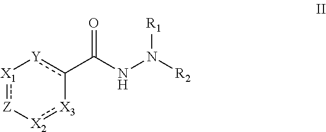

R₁ and R₂ are each independently selected from the group consisting of: H, substituted or unsubstituted C1-C6 alkyl, substituted or unsubstituted C6-C10 aryl, substituted or unsubstituted benzyl; R₁ and R₂, together with the N atom to which they attached form a 3-20 membered saturated or unsaturated carbocycle or heterocycle;
X₁ is selected from CR₃, N, and NH;
X₂ is selected from CR₄, and N;
X₃ is selected from CR₅, and N;
Y is selected from CR₆, NH, N, O, and S;
Z is selected from CₙR₇, N, and NH, where n is 0 or 1;
R₃, R₄, R₅, R₆, and R₇ are each independently H, halogen, hydroxyl, nitro, C₁₋₃ alkoxy (C₁₋₃ alkyl-O—), —NRaRb, oxo- (i.e. two hydrogen atoms on the same carbon atom on the group are replaced by ═O); wherein, Ra and Rb are each independently selected from the group consisting of: H, hydroxyl, amino, halogen, and C₁₋₄ alkyl;
or, any two of R₃, R₄, R₅, R₆, and R₇ located on adjacent ring atoms together with the carbon atom to which they attached form a substituted or unsubstituted C6-C10 aryl, or a substituted or unsubstituted 5-8 membered heteroaryl;
the “substituted” refers to one or more hydrogen atoms on the group being substituted with substituents selected from the group consisting of: C1-C6 alkyl, C1-C6 alkoxy, C6-C10 aryl, 3-20 membered heteroaryl, C6-C10 aryl-O—, halogen, nitro, hydroxyl, cyano, C2-C10 ester group (alkyl-COO—), C2-C10 acyl group (alkyl-CO—), sulfonate, oxo (i.e. two hydrogen atoms on the same carbon atom on the group are replaced by ═O), or two hydrogen atoms on two adjacent carbon atoms on the group are replaced by —(CH₂)n- (n is 1, 2, 3, 4, 5 or 6).

In another preferred embodiment, A is 3-12 membered saturated or unsaturated heterocyclyl, wherein the heterocyclyl contains 1-4 heteroatoms independently selected from N, O, and S.

In another preferred embodiment, A is 6-membered saturated heterocyclyl containing one N atom.

In another preferred embodiment, A is 5-membered saturated or unsaturated heterocyclyl, wherein the heterocyclyl contains 1-2 heteroatoms independently selected from N, O, and S.

In another preferred embodiment, the compound is selected from:

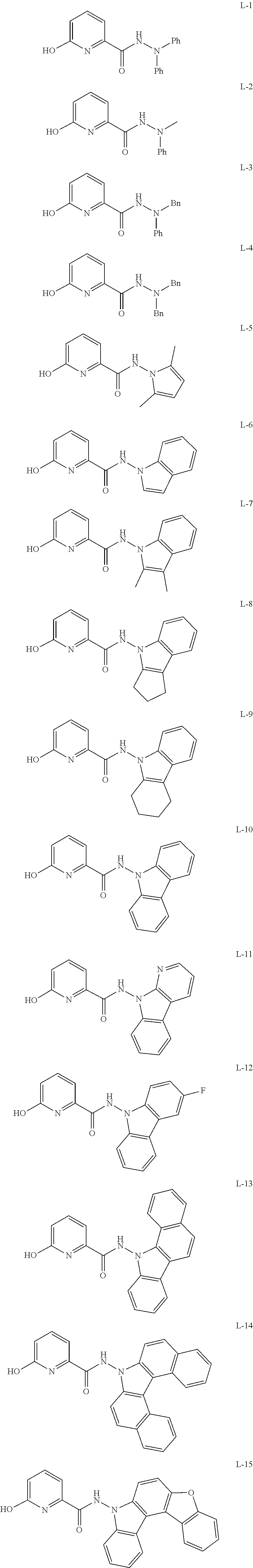

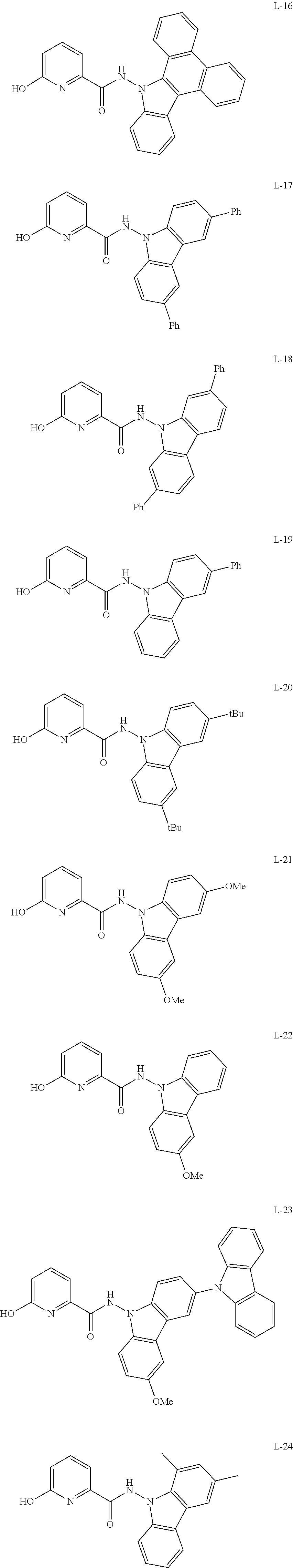

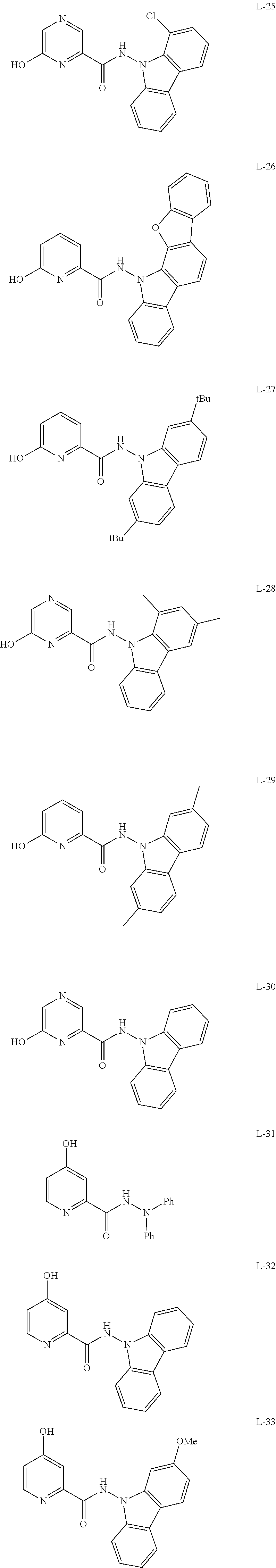

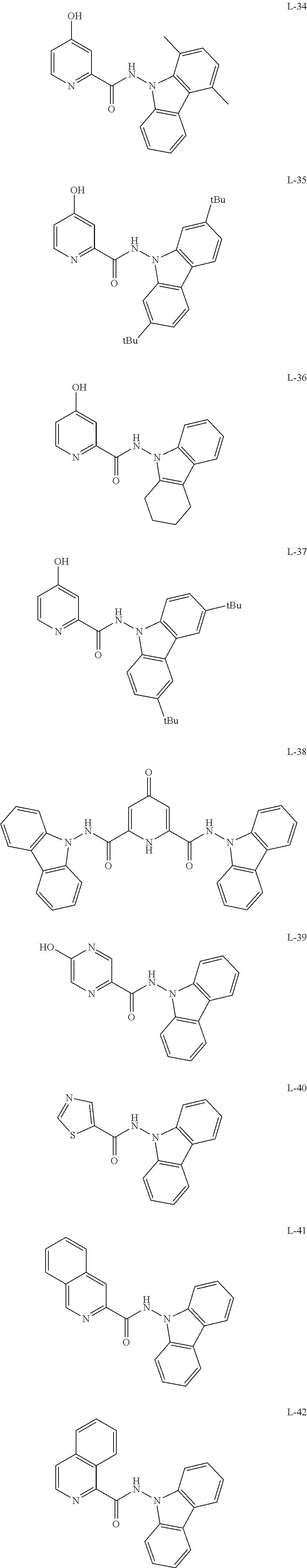

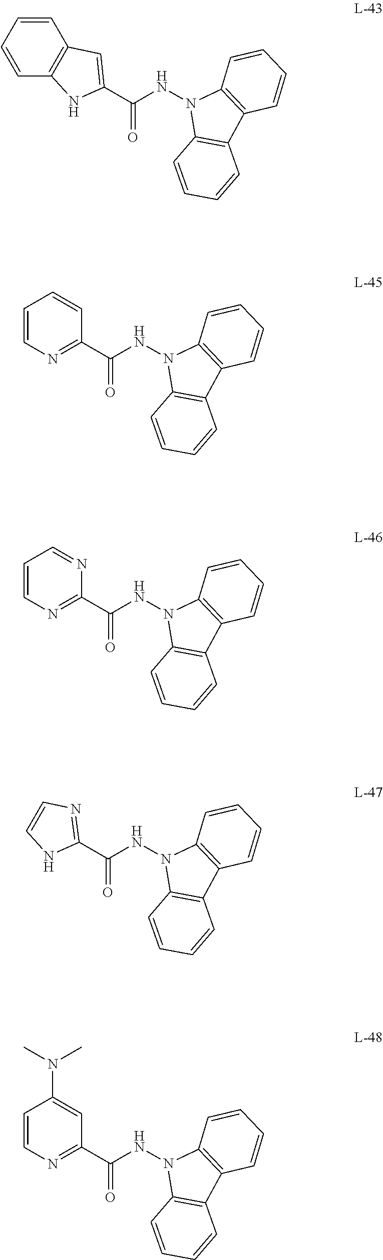 [SMILES: O=C(Nn1c2ccccc2c2ccccc21)c1cc2ccccc2[nH]1 ; O=C(Nn1c2ccccc2c2ccccc21)c1ccccn1 ; O=C(Nn1c2ccccc2c2ccccc21)c1ncccn1 ; O=C(Nn1c2ccccc2c2ccccc21)c1ncc[nH]1 ; CN(C)c1ccnc(C(=O)Nn2c3ccccc3c3ccccc32)c1]

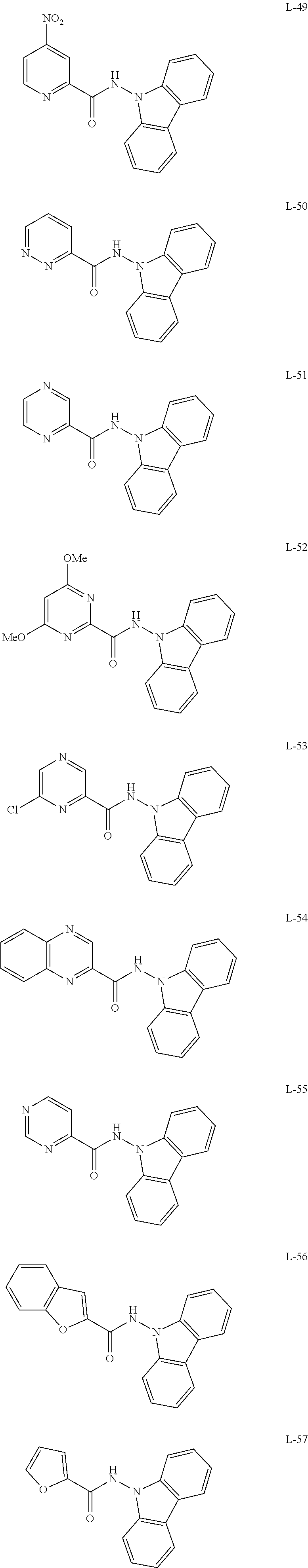

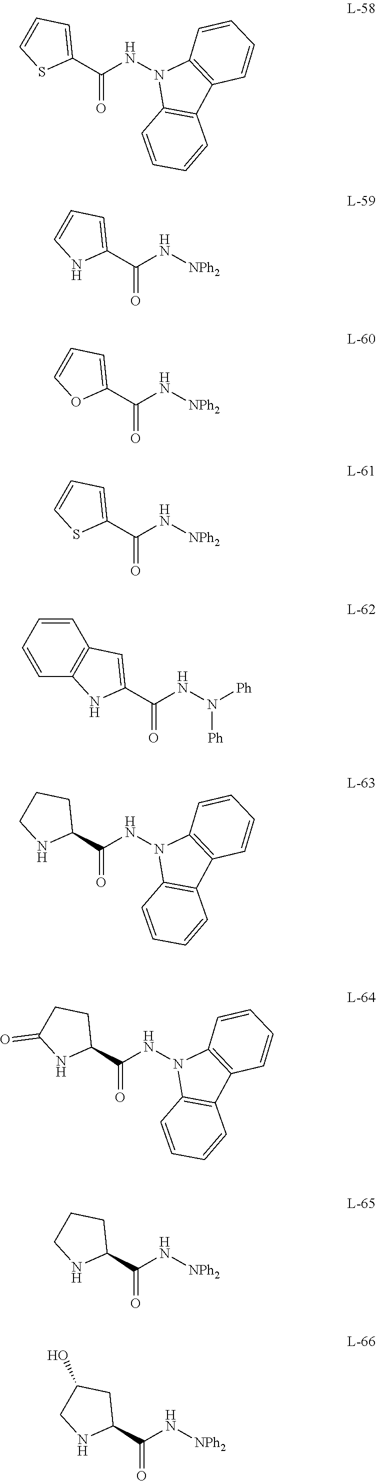

In the second aspect pf the present invention, provided is a method for coupling reaction of an aryl halide, comprising: using a copper catalyst and using a compound as shown in formula I as a ligand, carrying out a coupling reaction between the aryl halide and a coupling reagent:

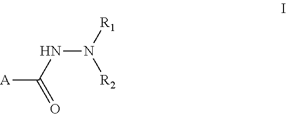

wherein, A, R₁, and R₂ are respectively as described in the first aspect of the present invention;
the aryl halide is selected from the group consisting of: aryl chloride, aryl bromide, or a combination thereof.

In another preferred embodiment, the reaction is carried out in the presence of a base.

In another preferred embodiment, the reaction comprises:

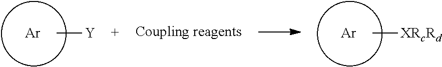

reacting

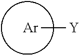

with a coupling reagent in an inert solvent to obtain a compound of

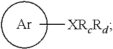

wherein, X is selected from the group consisting of: N, O, and S;
Y is selected from the group consisting of: Cl, Br, and I;

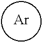

is selected from the group consisting of: substituted or unsubstituted C6-C20 aryl, substituted or unsubstituted 3-20 membered heteroaryl; wherein, the “substituted” refers to one or more hydrogen atoms in

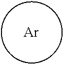

being substituted with substituents selected from the group consisting of: halogen, nitro, cyano, amino that is unsubstituted or substituted with one or two C1-C6 alkyl or C2-C10 acyl (alkyl-CO—), hydroxyl, unsubstituted or halogenated C1-C6 alkyl, C1-C6 alkoxy, C6-C10 aryl, 3-20 membered heteroaryl, C6-C10 aryl-O—, C2-C10 ester group (alkyl-COO—), C2-C10 acyl (alkyl-CO—), C2-C10 acyl-alkoxy (alkyl-OOC—), C2-C10 amide (alkyl-NHC(O)—, aryl-NHC(O)—), —COOH, hydroxy-C1-C10 alkylene, MeS—, sulfonyl and sulfonamide;
wherein, two hydrogen atoms on two adjacent carbon atoms in

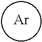

are replaced by —(CH₂)ₙ— (n is 1, 2, 3, 4, 5 or 6);
the coupling reagent is selected from the group consisting of: ammonia water, ammonia gas, ammonium salt (preferably ammonium chloride, ammonium carbonate, ammonium sulfate, ammonium hydrogen phosphate, or a combination thereof)/hydroxide solution (preferably potassium hydroxide solution),

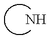

(the number of carbon atoms is 2-20), RₑC(O)NHR_{d}, R_{c}SO₂M (preferably, M is sodium or potassium), sodium azide, NHR_{c}R_{d}, R_{c}OH, R_{c}SH, hydroxides and salts that can hydrolyze to produce hydroxide, cyanides and cyanide salts that can ionize to form cyanide ions, aryl/heteroaryl terminal alkynes, alkyl terminal alkynes;
R_{c}, R_{d}, and Rₑ are each independently selected from the group consisting of: none, H, substituted or unsubstituted C1-C6 alkyl, substituted or unsubstituted C1-C6 alkenyl, substituted or unsubstituted C6-C20 aryl, substituted or unsubstituted 3- to 20-membered heteroaryl, substituted or unsubstituted C7-C25 alkyl-aryl, substituted or unsubstituted C1-C5 alkyl-3- to 20-membered heteroaryl, substituted or unsubstituted C3-C20 cycloalkyl, substituted or unsubstituted C1-C5 alkyl-C3-C20 cycloalkyl, substituted or unsubstituted 3- to 20-membered heterocyclyl, or substituted or unsubstituted C1-C5 alkyl-3- to 20-membered heterocyclyl;
or R_{c} and R_{d} together form a substituted or unsubstituted C3-C20 cycloalkyl, a substituted or unsubstituted 3- to 20-membered heterocyclyl;
or Rₑ and R_{d} together form a substituted or unsubstituted C3-C20 cycloalkyl, a substituted or unsubstituted 3- to 20-membered heterocyclyl;
wherein, the heteroaryl or heterocyclyl contains 1 to −5 heteroatoms selected from the group consisting of: N, O, and S; the cycloalkyl or heterocyclyl may be a monocyclic, polycyclic, spirocyclic, or bridged ring structure;
the “substituted” refers to one or more hydrogen atoms in the group being substituted with substituents selected from the group consisting of: halogen, cyano, oxo (i.e. two hydrogen atoms on the same carbon atom in the group are replaced by ═O), C1-C6 alkyl, halogenated C1-C6 alkyl, C1-C6 alkoxy, C6-C10 aryl, C6-C10 aryl-O—, C2-C10 ester group (alkyl-COO—), C2-C10 acyl-alkoxy (alkyl-OOC—), C2-C10 acyl (alkyl-CO—), C2-C10 amide (alkyl/aryl-NHC(O)—), —COOH, nitro, hydroxyl, amino, amino that is substituted by one or two C1-C6 alkyl, and C1-C6 alkyl-S—.

In another preferred embodiment, in the reaction, the molar ratio of the ligand to the reactant aryl halide is (0.01 to 50): 100, preferably is (0.01 to 5): 100; and/or
the molar ratio of the ligand to the copper catalyst is (1 to 5): 1, preferably is (1 to 2): 1.

In another preferred embodiment, the inert solvent is selected from the group consisting of: water, sulfone solvent, amide solvent, C1-C6 nitrile solvent, C2-C8 ether solvent, C1-C6 alcoholic solvent, C2-C8 ketone solvent, C2-C6 ester solvent, C6-C12 aromatic solvent, or combinations thereof.

In another preferred embodiment, the inert solvent is selected from the group consisting of: DMSO, DMF, DMA, NMP, acetonitrile, ethanol, ethylene glycol, tert butanol, polyethylene glycol-200, isopropanol, 2-methoxyethanol, toluene, methyl isobutyl ketone, propylene carbonate, water, sulfolane, 1-butanol, 1,4-dioxane, tetrahydrofuran, DME, toluene, or a combination thereof.

In another preferred embodiment, the reaction temperature is 40 to 180° C., preferably 40 to 120° C.

In another preferred embodiment, the reaction comprises the following (1), (2), (3), or (4):
(1) In an inert solvent, reacting

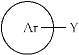

with NHR_{c}R_{d} to obtain

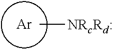

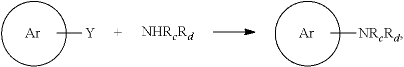

wherein, the definitions of each group are as described above;
(2) In an inert solvent, reacting

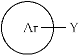

with H₂O to obtain

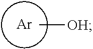

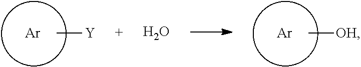

wherein, the definitions of each group are as described above;
(3) In an inert solvent, reacting

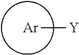

with R_{c}SO₂M to obtain

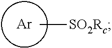

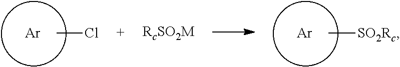

wherein, the definitions of each group are as described above.

In another preferred embodiment, in reaction (1), the ligand is preferably L1, L13, L16, L18, L24, L26, L27, and L29.

In another preferred embodiment, in reaction (1), the base is selected from potassium phosphate, potassium carbonate, cesium carbonate, sodium carbonate, lithium carbonate, sodium tert butoxide, potassium tert butoxide, triethylamine; preferably, potassium phosphate and potassium carbonate.

In another preferred embodiment, in reaction (1), the inert solvent is selected from the group consisting of: DMSO, Sulfolane, DMAc, NMP, DMI, tert butanol, 1,4-dioxane, 2-methoxyethanol, ethylene glycol, diethylene glycol, ethanol, PEG-200, or a combination thereof, preferably 2-methoxyethanol, diethylene glycol, PEG-200.

In another preferred embodiment, in reaction (1), the reaction temperature is 90 to 130° C., preferably 100° C.

In another preferred embodiment, in reaction (2), the ligand is preferably: L1, L5, L6, L7, L8, L9, L10, L11, L12, L13, L15, L16, L18, L20, L21, L22, L24, L25, L27, L28, L29, L32, orL39.

In another preferred embodiment, in reaction (2), the base is selected from the group consisting of: potassium carbonate, potassium phosphate, cesium carbonate, potassium hydroxide, sodium hydroxide, potassium tert butoxide, sodium tert butoxide, or combinations thereof, preferably potassium hydroxide and potassium tert butoxide.

In another preferred embodiment, in reaction (2), the water-mixed solvent is selected from the group consisting of: DMSO, DMA, DMF, 2-methoxyethanol, ethanol, THF, acetone, 1,4-dioxane, tert butanol, methanol, NMP, sulfolane, toluene, methyl isobutyl ketone, propylene carbonate, ethyl acetate, 1-butanol, ethylene glycol, and PEG-200.

In another preferred embodiment, in reaction (2), the copper salt is selected from CuBr, CuCl, CuI, Cu(acac)₂, Cu₂O, and Cu(OAc)₂, preferably, Cu₂O.

In another preferred embodiment, in reaction (2), the reaction temperature is 30 to 100° C.

In another preferred embodiment, reaction (2) has one or more of the following features:
(a) The reaction temperature is 30 to 45° C.; and/or
(b) The ligand is L−24, L-27, L-16, L-13, L-15, or L-10; preferably is L-24, or L-27; and/or
(c) The copper catalyst is Cu₂O; and/or
(d) The base is tBuOK, NaOH, KOH, or t-BuONa; and/or
(e) The solvent is selected from the group consisting of: polyethylene glycol, C1-C6 ketone solvents, C1-C6 alcoholic solvents, or a combination thereof; preferably is PEG-200, methyl isobutyl ketone, 2-methoxyethanol, or EtOH, or a combination thereof, and/or
(f) The molar ratio of copper salt to ligand is 1:(1±0.2).

In another preferred embodiment, in reaction (3), the ligand is preferably L32, L33, L34, L35, or L37.

In another preferred embodiment, in reaction (3), the copper catalyst is preferably CuBr, CuCl, CuI, CuTc, Cu, Cu₂O, CuCN, CuBr₂, Cu(OTf)₂, or Cu(acac)₂; Preferably is CuTc, CuBr, CuCl, CuI, or Cu.

In another preferred embodiment, in reaction (3), the reaction temperature is 120 to 140° C.

In another preferred embodiment, in reaction (3), R_{c}SO₂M is selected from the group consisting of: sodium C1-C6 alkyl sulfinate or sodium C6-C10 aryl sulfinate.

In another preferred embodiment, in reaction (3), R_{c}SO₂M is selected from sodium phenylsulfinate, sodium methylphenylsulfinate or sodium methanesulfinate.

In the third aspect of the present invention, provided is a catalytic system for aryl coupling reaction, comprising: copper catalyst, ligand, base, and inert solvent;
wherein, the copper catalyst is selected from the group consisting of: Cu, CuI, CuBr, CuCl, CuTc, Cu(OAc)₂, CuSO₄, Cu₂O, CuBr₂, CuCl₂, CuO, CuSCN, CuCN, Cu(OTf)₂, Cu(acac)₂, or a combination thereof,
the base is selected from the group consisting of: potassium carbonate, cesium carbonate, potassium phosphate, sodium bicarbonate, potassium bicarbonate, sodium carbonate, sodium hydroxide, potassium hydroxide, lithium hydroxide, sodium acetate, sodium methoxide, sodium ethoxide, sodium tert butoxide, potassium tert butoxide, or combinations thereof;
the inert solvent is selected from the group consisting of: DMSO, DMF, DMA, NMP, acetonitrile, ethanol, ethylene glycol, tert butanol, polyethylene glycol-200, isopropanol, 2-methoxyethanol, toluene, methyl isobutyl ketone, propylene carbonate, water, sulfolane, 1-butanol, acetone, 1,4-dioxane, tetrahydrofuran, DME, methanol, toluene, or combinations thereof;
the ligand has a structure shown in formula (I):

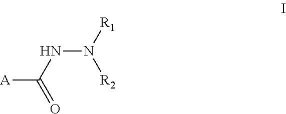

wherein, A, R₁, and R₂ are respectively as described in the first aspect of the present invention.

In the fourth aspect of the present invention, provided is a compound shown in formula I;

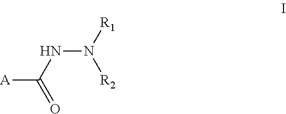

wherein, A is a saturated or unsaturated C5-C6 carbocyclyl, or a saturated or unsaturated 5- or 6-membered carbon heterocarbocyclyl, or a benzo saturated or unsaturated 5- or 6-membered carbon heterocarbocyclyl, wherein the carbon heterocarbocyclyl has 1 to 5 heteroatoms selected from the group consisting of: N, O, and S; A is optionally substituted by substituents selected from the group consisting of: hydroxyl, nitro, —NRaRb, halogen, C1-4 alkyl, C1-4 alkoxy, phenyl, and oxo- (i.e., two hydrogen atoms on the same carbon atom in the group are replaced by ═O);
Ra and Rb are each independently selected from the group consisting of: H, hydroxyl, amino, halogen, and C1-4 alkyl;
R₁ and R₂ are each independently selected from the group consisting of: H, substituted or unsubstituted C1-C6 alkyl, substituted or unsubstituted C1-C6 alkoxy, substituted or unsubstituted C3-C6 cycloalkyl, substituted or unsubstituted 3- to 6-membered heterocyclyl, substituted or unsubstituted C6-C20 aryl, substituted or unsubstituted 5- to 20-membered heteroaryl, substituted or unsubstituted benzyl; or, R₁ and R₂, together with the N atom to which they attached, form a 3-to 20-membered saturated or unsaturated carbocycle or heterocycle;
unless otherwise specified, the “substituted” refers to one or more hydrogen atoms in the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl (alkyl-CO—), sulfonate, or two hydrogen atoms on two adjacent carbon atoms in the group are replaced by —(CH₂)ₙ— (n is 1, 2, 3, 4, 5 or 6).

In another preferred embodiment, the compound of formula I is selected from the group consisting of:

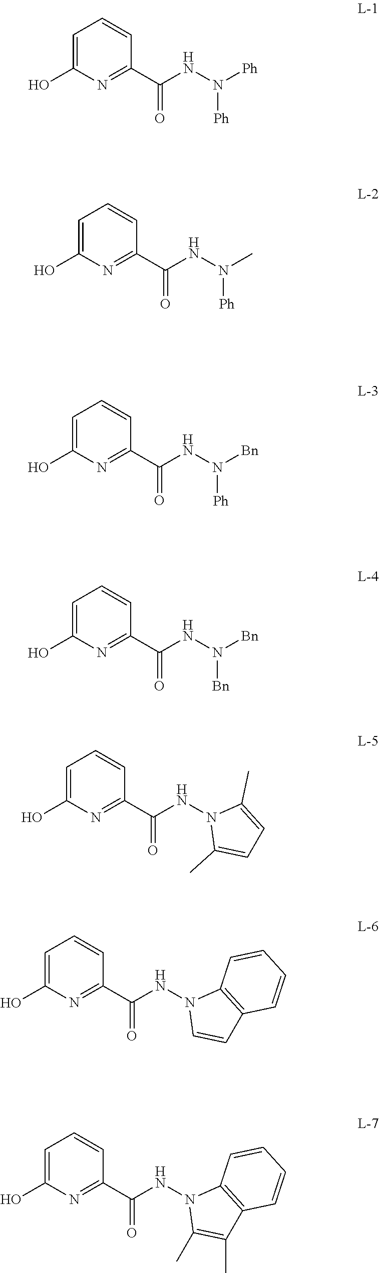

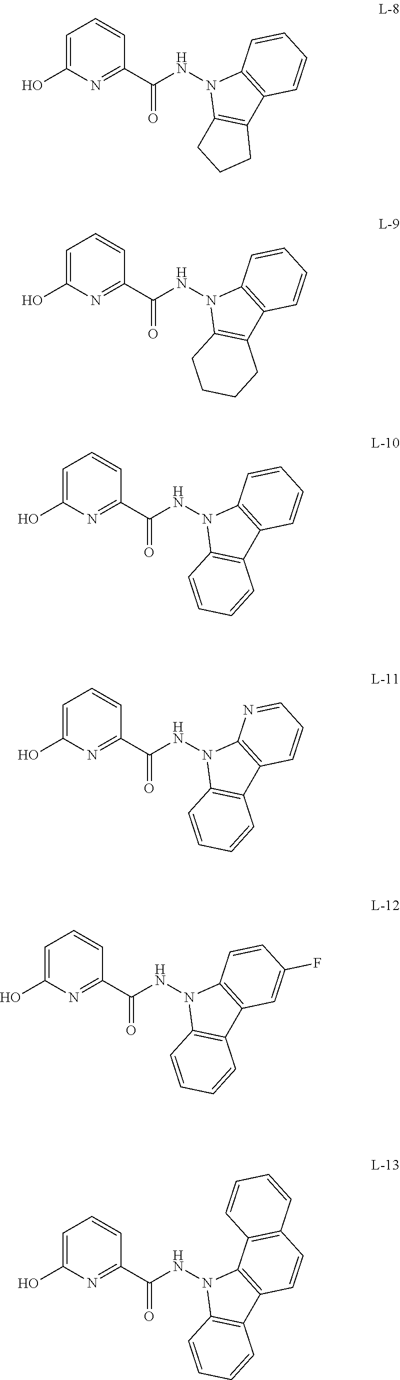 [SMILES: O=C(Nn1c2c(c3ccccc31)CCC2)c1cccc(O)n1 ; O=C(Nn1c2c(c3ccccc31)CCCC2)c1cccc(O)n1 ; O=C(Nn1c2ccccc2c2ccccc21)c1cccc(O)n1 ; O=C(Nn1c2ccccc2c2cccnc21)c1cccc(O)n1 ; O=C(Nn1c2ccccc2c2cc(F)ccc21)c1cccc(O)n1 ; O=C(Nn1c2ccccc2c2ccc3ccccc3c21)c1cccc(O)n1]

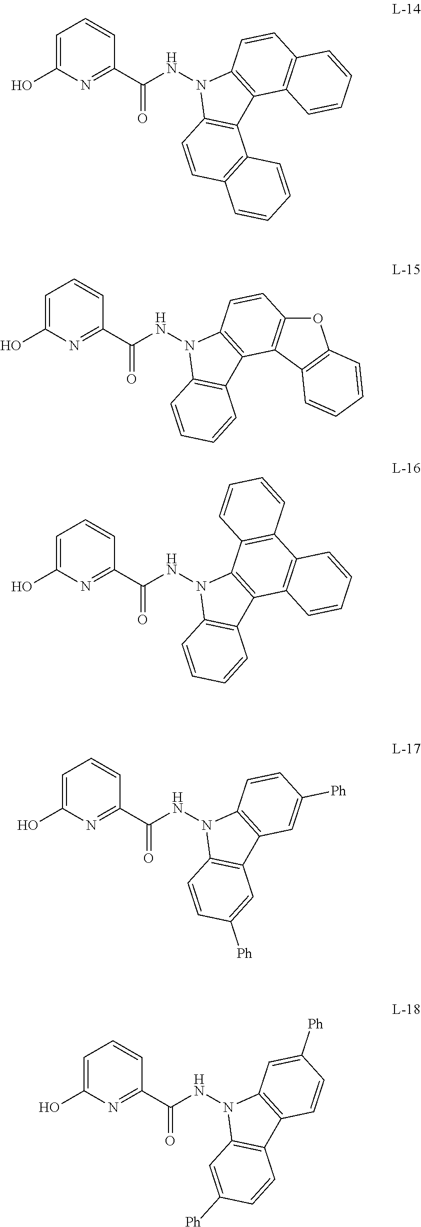

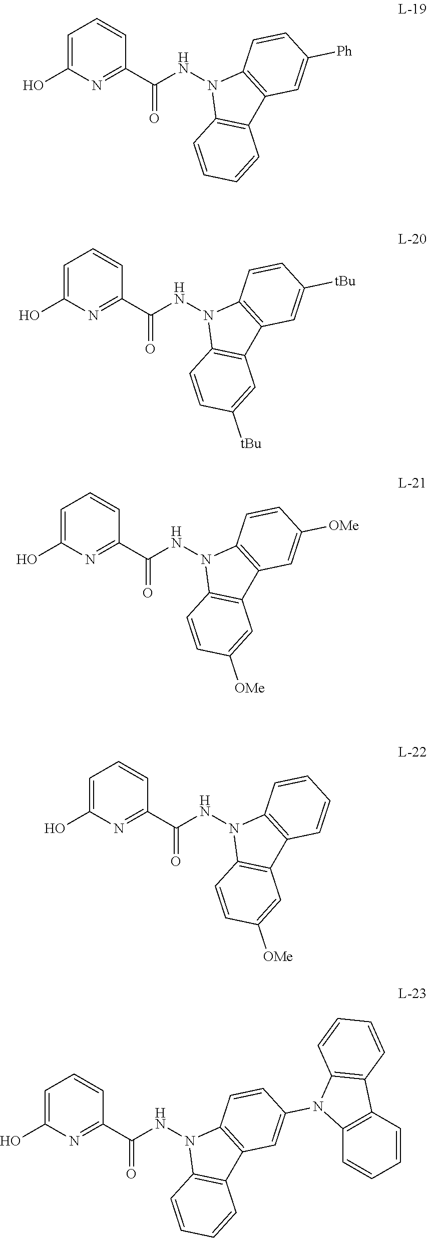 [SMILES: O=C(Nn1c2ccccc2c2cc(-c3ccccc3)ccc21)c1cccc(O)n1 ; CC(C)(C)c1ccc2c(c1)c1cc(C(C)(C)C)ccc1n2NC(=O)c1cccc(O)n1 ; COc1ccc2c(c1)c1cc(OC)ccc1n2NC(=O)c1cccc(O)n1 ; COc1ccc2c(c1)c1ccccc1n2NC(=O)c1cccc(O)n1 ; O=C(Nn1c2ccccc2c2cc(-n3c4ccccc4c4ccccc43)ccc21)c1cccc(O)n1]

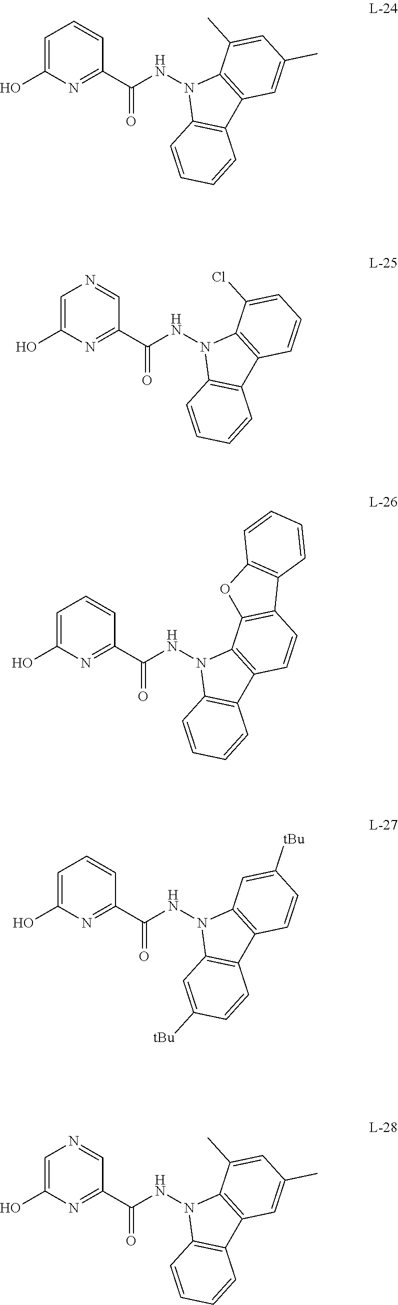 [SMILES: Cc1cc(C)c2c(c1)c1ccccc1n2NC(=O)c1cccc(O)n1 ; O=C(Nn1c2ccccc2c2cccc(Cl)c21)c1cncc(O)n1 ; O=C(Nn1c2ccccc2c2ccc3c4ccccc4oc3c21)c1cccc(O)n1 ; CC(C)(C)c1ccc2c3ccc(C(C)(C)C)cc3n(NC(=O)c3cccc(O)n3)c2c1 ; Cc1cc(C)c2c(c1)c1ccccc1n2NC(=O)c1cncc(O)n1]

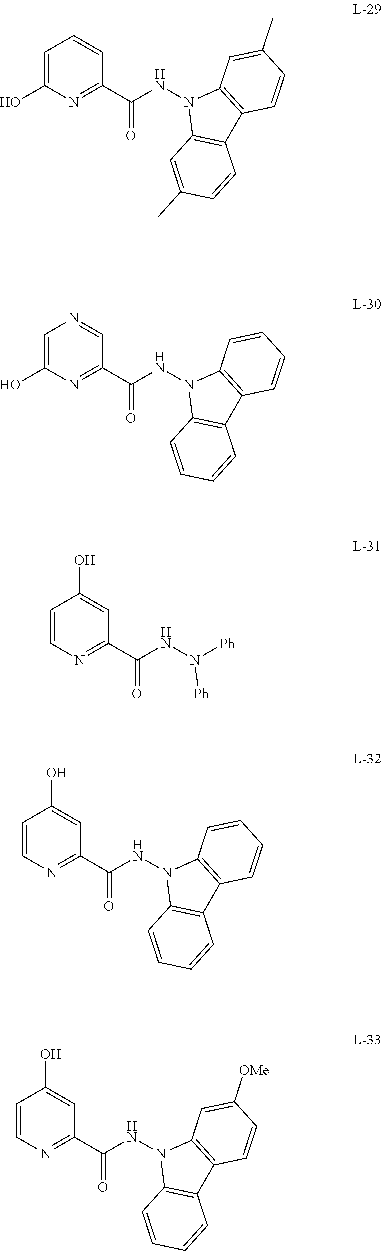 [SMILES: Cc1ccc2c3ccc(C)cc3n(NC(=O)c3cccc(O)n3)c2c1 ; O=C(Nn1c2ccccc2c2ccccc21)c1cncc(O)n1 ; O=C(NN(c1ccccc1)c1ccccc1)c1cc(O)ccn1 ; O=C(Nn1c2ccccc2c2ccccc21)c1cc(O)ccn1 ; COc1ccc2c3ccccc3n(NC(=O)c3cc(O)ccn3)c2c1]

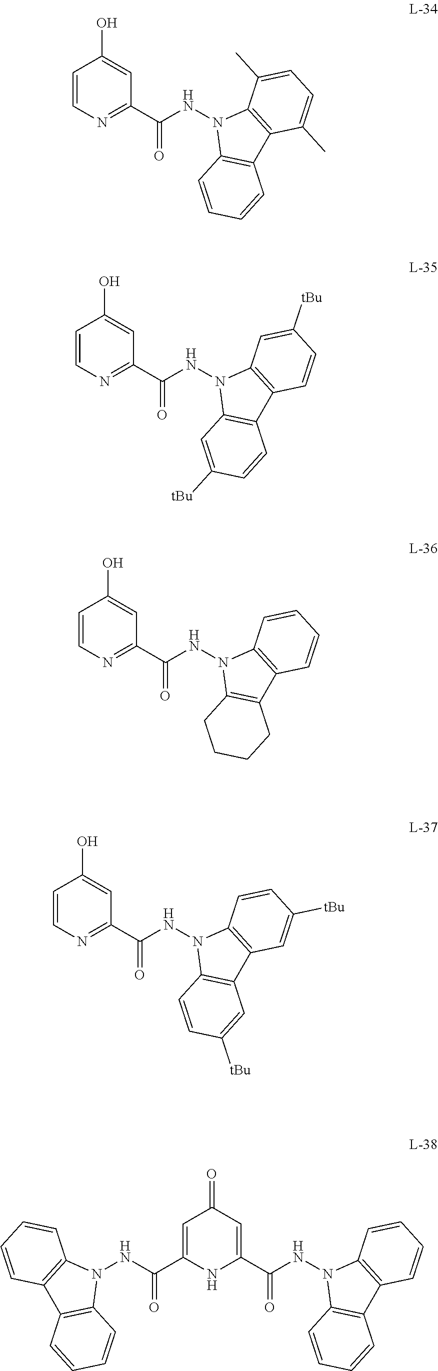 [SMILES: Cc1ccc(C)c2c1c1ccccc1n2NC(=O)c1cc(O)ccn1 ; CC(C)(C)c1ccc2c3ccc(C(C)(C)C)cc3n(NC(=O)c3cc(O)ccn3)c2c1 ; O=C(Nn1c2c(c3ccccc31)CCCC2)c1cc(O)ccn1 ; CC(C)(C)c1ccc2c(c1)c1cc(C(C)(C)C)ccc1n2NC(=O)c1cc(O)ccn1 ; O=C(Nn1c2ccccc2c2ccccc21)c1cc(=O)cc(C(=O)Nn2c3ccccc3c3ccccc32)[nH]1]

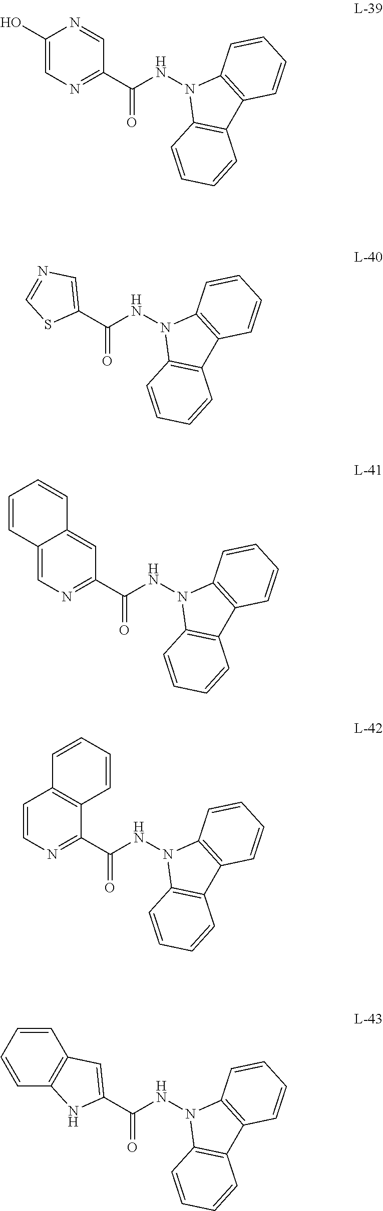 [SMILES: O=C(Nn1c2ccccc2c2ccccc21)c1cnc(O)cn1 ; O=C(Nn1c2ccccc2c2ccccc21)c1cncs1 ; O=C(Nn1c2ccccc2c2ccccc21)c1cc2ccccc2cn1 ; O=C(Nn1c2ccccc2c2ccccc21)c1nccc2ccccc12 ; O=C(Nn1c2ccccc2c2ccccc21)c1cc2ccccc2[nH]1]

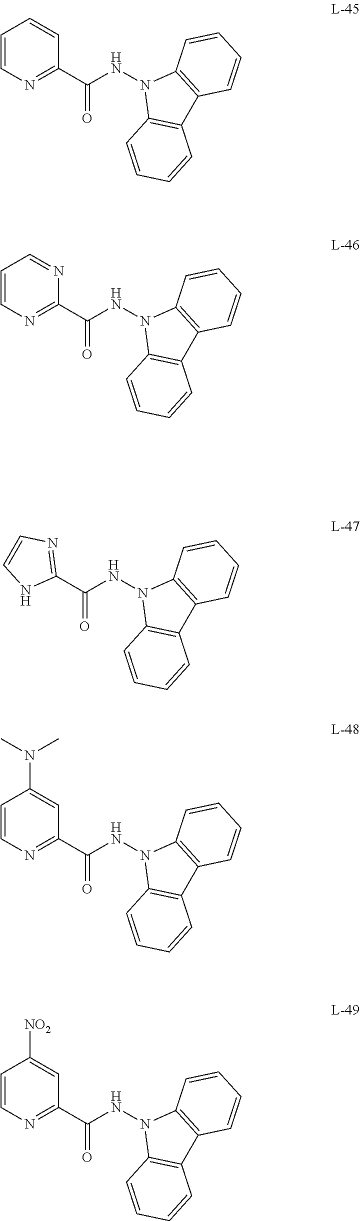 [SMILES: O=C(Nn1c2ccccc2c2ccccc21)c1ccccn1 ; O=C(Nn1c2ccccc2c2ccccc21)c1ncccn1 ; O=C(Nn1c2ccccc2c2ccccc21)c1ncc[nH]1 ; CN(C)c1ccnc(C(=O)Nn2c3ccccc3c3ccccc32)c1 ; O=C(Nn1c2ccccc2c2ccccc21)c1cc([N+](=O)[O-])ccn1]

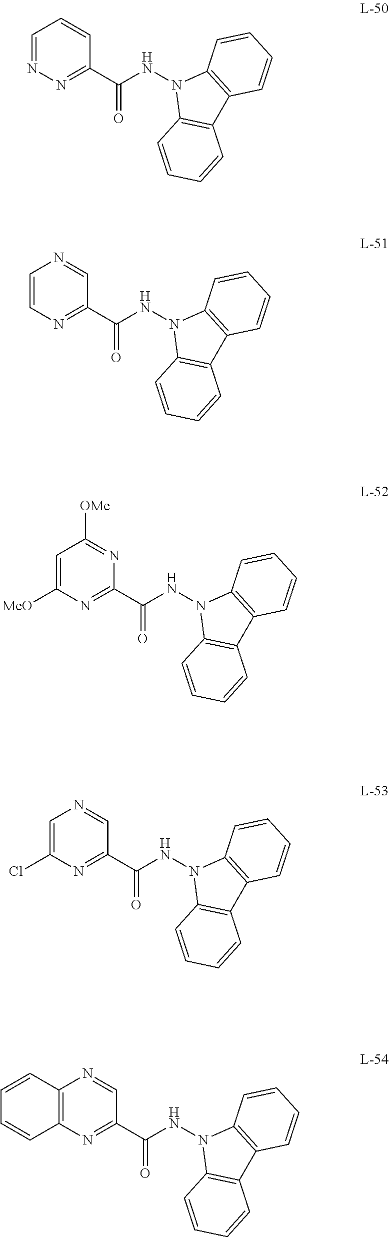 [SMILES: O=C(Nn1c2ccccc2c2ccccc21)c1cccnn1 ; O=C(Nn1c2ccccc2c2ccccc21)c1cnccn1 ; COc1cc(OC)nc(C(=O)Nn2c3ccccc3c3ccccc32)n1 ; O=C(Nn1c2ccccc2c2ccccc21)c1cncc(Cl)n1 ; O=C(Nn1c2ccccc2c2ccccc21)c1cnc2ccccc2n1]

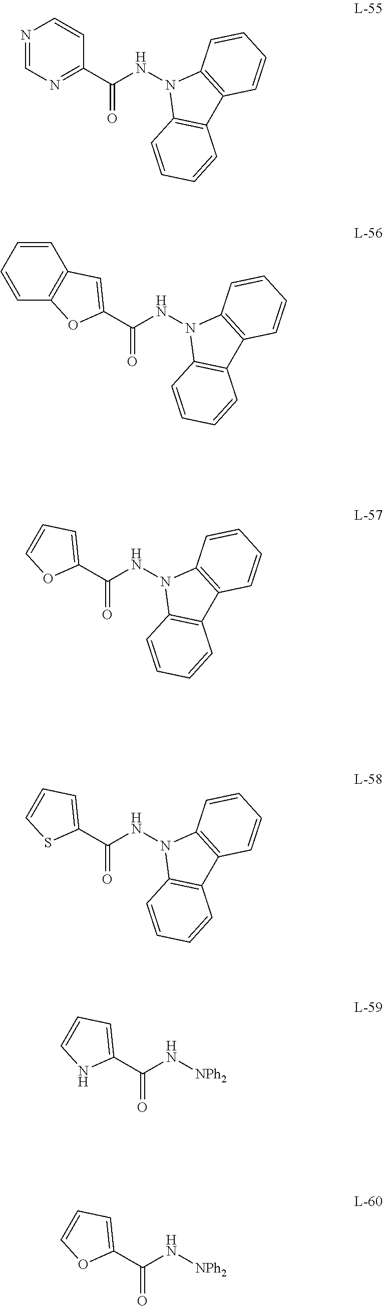

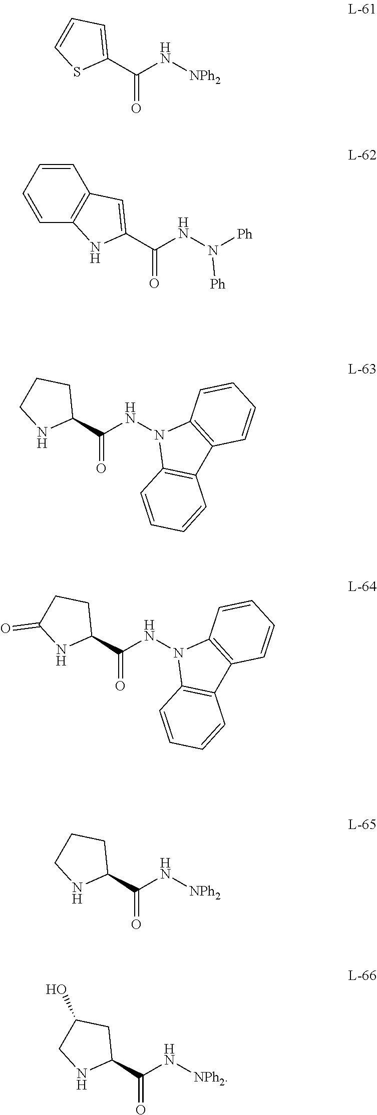

It should be understood that within the scope of the present invention, the above-mentioned technical features of the present invention and the specific technical features described in the following embodiments can be combined with each other to form a new or preferred technical solution. Due to space limitations, it will not be elaborated here.

### DETAILED DESCRIPTION OF THE INVENTION

After long-term and in-depth research, the inventor has provided a class of heterocyclic carboxylic acid hydrazide ligands suitable for copper catalyzed coupling reactions of aryl chloride/bromide. A suitable catalytic system comprising the ligands, copper reagents, bases, and solvents can be used for copper-catalyzed coupling reactions of aryl halides, especially for effectively promoting the copper-catalyzed coupling of aryl chloride/bromide with various nucleophiles, which is difficult to occur under conventional conditions, to generate C—N, C—O, C—S, C—C bonds. Furthermore, the catalytic system can effectively lower the reaction temperature, reduce the amount of catalyst and ligand, and synthesize numerous useful small molecule compounds. The method has mild reaction conditions, low catalyst ligand dosage, wide application range and good industrial application prospects.

### Term

As used herein, the term “halogen” refers to fluorine, chlorine, bromine, or iodine.

The term “halogenated” refers to one or more hydrogen atoms in a group being replaced by halogens.

The term “alkyl” refers to straight or branched alkyl groups. When there is a limit on the number of carbon atoms (such as C1-C6) in front of the alkyl group, it means that the alkyl contains 1 to 6 carbon atoms, such as methyl, ethyl, propyl, isopropyl, butyl, isobutyl, sec-butyl, tert-butyl, or similar groups.

The term “cycloalkyl” refers to a bicyclic or tricyclic (fused, bridged, or spiro) ring system with saturated or partially saturated units. The cycloalkyl may have 3 to 20 carbon atoms. When there is a limit on the number of carbon atoms (e.g., C3-C20) before the cycloalkyl group, it means that the cycloalkyl contains 3 to 20 carbon atoms, such as cyclopropyl, cyclobutyl, cyclopentyl, cycloheptyl, or the like. The cycloalkyl can be in the form of a monocyclic, polycyclic, spirocyclic, or bridged ring.

As used herein, the term “alkoxy” refers to an alkyl group connected by an oxygen atom (e.g., —O-alkyl, wherein the alkyl group is as described above), such as (but not limited to) methoxy, ethoxy, propoxy, isopropoxy, butoxy, isobutoxy, sec-butoxy, tert-butoxy, or similar groups. When there is a limit on the number of carbon atoms (such as C1-C6) before the alkoxy, it means that the cycloalkyl contains 1 to 6 carbon atoms.

The term “aryl” refers to a monocyclic, bicyclic, or fused aromatic hydrocarbon group, which can be substituted or unsubstituted. When there is a limit on the number of carbon atoms (such as C6-C20) before an aryl, it means that the aryl contains 6 to 20 carbon atoms. Examples of aryl include (but are not limited to): phenyl, biphenyl, naphthyl, or similar groups (wherein each carbon atom can be arbitrarily substituted).

The term “heteroaryl” refers to a monocyclic, bicyclic, or fused aromatic group comprising at least one heteroatom selected from N, O, and S. The heteroaryl can be a 3- to 20 membered aromatic ring group containing 1-5 heteroatoms independently selected from N, O, and S. Examples of heteroaryl include (but are not limited to) pyridine, pyrimidine, pyrrole, indazole, indole, furane, benzofurane, thiophene, or similar groups.

The term “heterocyclyl” refers to a monocyclic or fused saturated or partially saturated substituent, which includes at least one identical or different heteroatoms selected from N, O, and S. The heterocyclyl can be a 3- to 20 membered heterocyclyl having 1 to 5 heteroatoms independently selected from N, O, and S. Examples of heterocyclyl include (but are not limited to): nitrogen-containing heterocyclyl, oxygen-containing heterocyclyl, sulfur-containing heterocyclyl, nitrogen- and oxygen-containing heterocyclyl, etc.

The term “ester group” refers to a group having a structure of “alkyl-COO-”, wherein the alkyl is defined as described above.

The term “acyl” refers to a group having a structure of “alkyl-CO-”, wherein the alkyl is defined as described above.

The term “amido” refers to a functional group having a structure of “alkyl-NHC(O)—” or “aryl-NHC(O)—”, wherein alkyl and aryl are defined as above.

### Ligand

Unless otherwise specified, the ligands mentioned herein refer to ligands used in copper catalyzed coupling reactions of aryl chloride/bromide.

The ligands available in the present invention have a structure as shown in formula (I) above. In a preferred embodiment of the present invention, the ligands have the following structure (the definitions of each functional group are as described above):

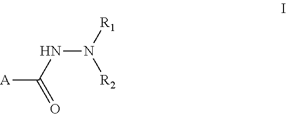

wherein, A is a saturated or unsaturated C5-C6 carbocyclyl, or a saturated or unsaturated 5- or 6-membered carbon heterocarbocyclyl, or a benzo saturated or unsaturated 5- or 6-membered carbon heterocarbocyclyl, wherein the carbon heterocarbocyclyl has 1 to 5 heteroatoms selected from the group consisting of: N, O, and S; A is optionally substituted by substituents selected from the group consisting of: hydroxyl, nitro, —NRaRb, halogen, C1-4 alkyl, C1-4 alkoxy, phenyl, and oxo- (i.e., two hydrogen atoms on the same carbon atom in the group are replaced by ═O);
Ra and Rb are each independently selected from the group consisting of: H, hydroxyl, amino, halogen, and C1-4 alkyl;
R₁ and R₂ are each independently selected from the group consisting of: H, substituted or unsubstituted C1-C6 alkyl, substituted or unsubstituted C1-C6 alkoxy, substituted or unsubstituted C3-C6 cycloalkyl, substituted or unsubstituted 3- to 6-membered heterocyclyl, substituted or unsubstituted C6-C20 aryl, substituted or unsubstituted 5- to 20-membered heteroaryl, substituted or unsubstituted benzyl; or, R₁ and R₂, together with the N atom to which they attached, form a 3-to 20-membered saturated or unsaturated carbocycle or heterocycle (such as substituted or unsubstituted pyrrole, indole, azoles, benzazole, and aromatic heterocycle);
as a ligand for copper-catalyzed coupling reactions of aryl halide; wherein, the aryl halide is selected from the group consisting of: aryl chloride, aryl bromide, aryl iodide, or a combination thereof;
unless otherwise specified, the “substituted” refers to one or more hydrogen atoms in the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl (alkyl-CO—), sulfonate, or two hydrogen atoms on two adjacent carbon atoms in the group are replaced by —(CH₂)ₙ— (n is 1, 2, 3, 4, 5 or 6).

Preferably, A is selected from the group consisting of: substituted or unsubstituted indolyl, substituted or unsubstituted benzimidazolyl, substituted or unsubstituted imidazolyl, substituted or unsubstituted thiazolyl, substituted or unsubstituted benzothiazolyl, substituted or unsubstituted thienyl, substituted or unsubstituted benzothienyl, substituted or unsubstituted furyl, substituted or unsubstituted benzofuranyl, substituted or unsubstituted saturated or unsaturated 5- to 6-membered heterocyclyl containing 1 or 2 N atoms (such as pyridyl, pyrazinyl, pyrimidinyl, pyridazinyl, azolyl, tetrahydropyrrolyl, hexahydropiperidinyl), substituted or unsubstituted phenyl, substituted or unsubstituted naphthyl, substituted or unsubstituted benzyl, substituted or unsubstituted quinolinyl, substituted or unsubstituted isoquinolinyl;
the “substituted” refers to one or more hydrogen atoms being substituted with substituents selected from the group consisting of: hydroxyl, nitro, —NRaRb, halogen, C1-4 alkyl, C1-4 alkoxy, phenyl, and oxo (i.e., two hydrogen atoms on the same carbon atom in the group are replaced by ═O), wherein, Ra and Rb are each independently selected from the group consisting of: H, hydroxyl, amino, halogen, and C1-4 alkyl.

Preferably, R₁ and R₂ are each independently selected from the group consisting of: H, substituted or unsubstituted C1-C6 alkyl, substituted or unsubstituted C1-C6 alkoxy, substituted or unsubstituted C6-C10 aryl, substituted or unsubstituted 5- to 10-membered heteroaryl, substituted or unsubstituted benzyl; or, R₁ and R₂, together with the N atom to which they attached, form a 3- to 20-membered substituted or unsubstituted saturated or unsaturated carbocycle or heterocycle.

More preferably, R₁ and R₂ are each independently selected from the group consisting of: H, substituted or unsubstituted C1-C6 alkyl, substituted or unsubstituted phenyl, substituted or unsubstituted benzyl; or, R₁ and R₂, together with the N atom to which they attached, form a 3- to 20-membered carbocycle or heterocycle selected from the group consisting of: substituted or unsubstituted pyrrole, substituted or unsubstituted indole, substituted or unsubstituted azoles, substituted or unsubstituted benzazole, and substituted or unsubstituted aromatic heterocycle.

In a preferred embodiment, the structure of the compound is as shown in formula II below:

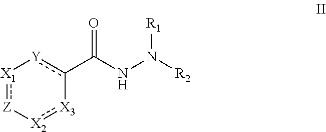

R₁ and R₂ are each independently selected from the group consisting of: H, substituted or unsubstituted C1-C6 alkyl, substituted or unsubstituted C6-C10 aryl, substituted or unsubstituted benzyl; R₁ and R₂, together with the N atom to which they attached form a 3- to 20-membered saturated or unsaturated carbocycle or heterocycle;
X₁ is selected from CR₃, N, and NH;
X₂ is selected from CR₄, and N;
X₃ is selected from CR₅, and N;
Y is selected from CR₆, NH, N, O, and S;
Z is selected from CₙR₇, N, and NH, where n is 0 or 1;
R₃, R₄, R₅, R₆, and R₇ are each independently H, halogen, hydroxyl, nitro, C₁₋₃ alkoxy (C₁₋₃ alkyl-O—), —NRaRb, oxo- (i.e. two hydrogen atoms on the same carbon atom in the group are replaced by ═O); wherein, Ra and Rb are each independently selected from the group consisting of: H, hydroxyl, amino, halogen, and C₁₋₄ alkyl;
or, any two of R₃, R₄, R₅, R₆, and R₇ located on adjacent ring atoms together with the carbon atom to which they attached form a substituted or unsubstituted C₆-C₁₀ aryl, or a substituted or unsubstituted 5- to 8-membered heteroaryl;
the “substituted” refers to one or more hydrogen atoms in the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl group (alkyl-CO—), sulfonate, oxo (i.e. two hydrogen atoms on the same carbon atom in the group are replaced by ═O), or two hydrogen atoms on two adjacent carbon atoms in the group are replaced by —(CH₂)ₙ— (n is 1, 2, 3, 4, 5 or 6).

Preferably, A is 3- to 12-membered saturated or unsaturated heterocyclyl, wherein the heterocyclyl contains 1 to 4 heteroatoms independently selected from N, O, and S.

Preferably, A is 6-membered saturated heterocyclyl containing one N atom.

Preferably, A is 5-membered saturated or unsaturated heterocyclyl, wherein the heterocycle contains 1 to 2 heteroatoms independently selected from N, O, and S.

Each of the above-mentioned ligands can be obtained through commercial channels or prepared according to the preferred method provided by the present invention.

It should be understood that since the bond energy of the C—I bond is smaller than that of the C—Cl/Br bond, under the same conditions, the coupling reaction of aryl iodides is more likely to occur than that of aryl chlorides/bromides, the amount of catalyst and ligand required is lower, and the conditions are milder. Therefore, the above ligands can be used not only for the coupling reaction of aryl chlorides, but also for the coupling reaction of conventional aryl iodides in this field.

### Copper Catalyzed Coupling Reactions of Aryl Chloride/Bromide Compounds

The present invention also provides a kind of copper-catalyzed coupling reaction method for aryl chloride/bromide compounds, which comprises carrying the above reaction by using the compound of formula (I) as a ligand as described previously.

Generally, aryl iodides/bromides have higher reactivity and can achieve corresponding coupling reactions well under the catalysis of transition metals such as palladium, copper, and nickel. However, the C—Cl bond has high bond energy, which makes it difficult for aryl chlorides to react under conventional catalytic conditions for aryl iodides/bromides, requiring higher reaction temperatures and larger dosage of catalyst and ligand. Secondly, although the reactivity of aryl bromides is higher than that of chlorides, copper-catalyzed coupling reactions under ultra-low catalyst dosage conditions (0.01 mol %) are still rare. Compared with iodinated aromatic hydrocarbons, chlorinated/brominated aromatic hydrocarbons are cheaper and more commercially available. An efficient coupling reaction of aryl chlorides/bromides (with low reaction temperature, low catalyst and ligand dosage, good substrate universality) has greater application prospects in medicinal chemistry, fine chemical engineering, and the production of bulk chemicals.

For different reactants, the ligand and reaction conditions can be optimized within the disclosure of the present invention, so as to select the most suitable ligand type and reaction conditions (such as temperature, solvent, ratio of reactants, reaction time, etc.). After reading the disclosed content of the present application, the above optimization is within the skill of those skilled in the art.

The most preferred copper catalyzed coupling reactions of aryl chloride are as follows:

### 1. Heterocyclic Carboxylic Acid Hydrazide Ligand-Promoted Copper-Catalyzed C—N Coupling Reaction of Aryl Chlorides

In the above reactions, the selection of the coupling reagent has no specific limitation and can be corresponding primary or secondary amines. The specific reaction process is as follows:

In the case that the coupling reagent is a primary or secondary amine, the reaction is as follows:

In an inert solvent, reacting

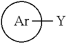

with NHR_{c}R_{d} to obtain

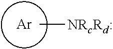

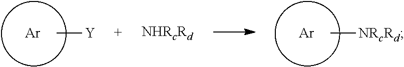

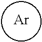

is selected from the group consisting of: substituted or unsubstituted C6-C20 aryl, substituted or unsubstituted 3-20 membered heteroaryl; wherein, the “substituted” refers to one or more hydrogen atoms in

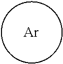

being substituted with substituents selected from the group consisting of: halogen, nitro, cyano, amino that is unsubstituted or substituted with one or two C1-C6 alkyl or C2-C10 acyl (alkyl-CO—), hydroxyl, unsubstituted or halogenated C1-C6 alkyl, C1-C6 alkoxy, C6-C10 aryl, 3- to 20-membered heteroaryl, C6-C10 aryl-O—, C2-C10 ester group (alkyl-COO—), C2-C10 acyl (alkyl-CO—), C2-C10 acyl-alkoxy (alkyl-OOC—), C2-C10 amido (alkyl-NHC(O)—, aryl NHC(O)—), —COOH, hydroxy-C1-C10 alkylene, MeS—, sulfonyl and sulfonamide;
wherein, two hydrogen atoms on two adjacent carbon atoms in

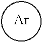

can be replaced by —(CH₂)ₙ— (n is 1, 2, 3, 4, 5 or 6);
R_{c} and R_{d} are each independently selected from the group consisting of: H, substituted or unsubstituted C1-C6 alkyl, substituted or unsubstituted C1-C6 alkenyl, substituted or unsubstituted C6-C20 aryl, substituted or unsubstituted 3- to 20-membered heteroaryl, substituted or unsubstituted C7-C25 alkyl-aryl, substituted or unsubstituted C1-C5 alkyl-3- to 20-membered heteroaryl, substituted or unsubstituted C3-C20 cycloalkyl, substituted or unsubstituted C1-C5 alkyl-C3-C20 cycloalkyl, substituted or unsubstituted 3- to 20-membered heterocyclyl, or substituted or unsubstituted C1-C5 alkyl-3- to 20-membered heterocyclyl; or, R_{c} and R_{d} together form a substituted or unsubstituted C3-C20 cycloalkyl or a substituted or unsubstituted 3- to 20-membered heterocyclyl.

There is no particular limitation on the ligand, and the ligand can be any of the ligands mentioned above, preferably L1, L13, L16, L18, L24, L26, L27, |L29.

Preferable bases can be potassium phosphate, potassium carbonate, cesium carbonate, sodium carbonate, lithium carbonate, sodium tert-butoxide, potassium tert-butoxide, triethylamine; preferably, potassium phosphate, or potassium carbonate.

Preferable inert solvents can be DMSO, sulfolane, DMAc, NMP, DMI, tert-butanol, 1,4-dioxane, 2-methoxyethanol, ethylene glycol, diethylene glycol, ethanol, PEG-200, or a combination thereof, preferably 2-methoxyethanol, diethylene glycol, PEG-200.

The reaction temperature is 100-125° C., preferably is 100° C.

### 2. Heterocyclic Carboxylic Acid Hydrazide Ligand-Promoted Copper-Catalyzed C—O Coupling Reaction of Aryl Chlorides

In an inert solvent, reacting

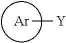

with H₂O to obtain

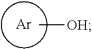

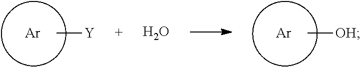

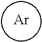

is selected from the group consisting of: substituted or unsubstituted C6-C20 aryl, substituted or unsubstituted 3- to 20-membered heteroaryl; wherein, the “substituted” refers to one or more hydrogen atoms in

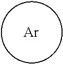

being substituted with substituents selected from the group consisting of: halogen, nitro, cyano, amino that is unsubstituted or substituted with one or two C1-C6 alkyl or C2-C10 acyl (alkyl-CO—), hydroxyl, unsubstituted or halogenated C1-C6 alkyl, C1-C6 alkoxy, C6-C10 aryl, 3- to 20-membered heteroaryl, C6-C10 aryl-O—, C2-C10 ester group (alkyl-COO—), C2-C10 acyl (alkyl-CO—), C2-C10 acyl-alkoxy (alkyl-OOC—), C2-C10 amido (alkyl-NHC(O)—, aryl NHC(O)—), —COOH, hydroxy-C1-C10 alkylene, MeS—, sulfonyl and sulfonamide;
wherein, two hydrogen atoms on two adjacent carbon atoms in

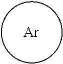

can be replaced by —(CH₂)ₙ— (n is 1, 2, 3, 4, 5 or 6);

There is no particular limitation on the ligand, and the ligand can be any of the ligands mentioned above, preferably L1, L5, L6, L7, L8, L9, L10, L11, L12, L13, L15, L16, L18, L20, L21, L22, L24, L25, L27, L28, L29, L32, L39; more preferably L-24, L-27, L-16, L-13, L-15, L-10; most preferably L-24 or L-27.

The copper catalyst can be CuBr, CuCl, CuI, Cu(acac)₂, Cu₂O, Cu(OAc)₂; preferably Cu₂O.

Base can be potassium carbonate, potassium phosphate, cesium carbonate, potassium hydroxide, sodium hydroxide, potassium tert-butoxide, sodium tert-butoxide, or a combination thereof, preferably potassium hydroxide or potassium tert-butoxide.

Water mixed solvents can be DMSO, DMA, DMF, 2-methoxyethanol, ethanol, THF, acetone, 1,4-dioxane, tert-butanol, methanol, NMP, sulfolane, toluene, methyl isobutyl ketone, propylene carbonate, ethyl acetate, 1-butanol, ethylene glycol, or PEG-200.

The reaction temperature is 30-100° C.; preferably 30-45° C.

The molar ratio of copper salt to ligand is 1:(1±0.2).

### 3. Heterocyclic Carboxylic Acid Hydrazide Ligand-Promoted Copper-Catalyzed C—S Coupling Reaction of Aryl Chlorides

In an inert solvent, reacting

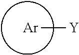

with R_{c}SO₂M to obtain

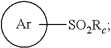

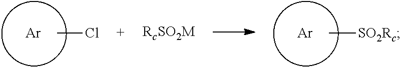

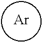

is selected from the group consisting of: substituted or unsubstituted C6-C20 aryl, substituted or unsubstituted 3- to 20-membered heteroaryl; wherein, the “substituted” refers to one or more hydrogen atoms in

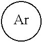

being substituted with substituents selected from the group consisting of: halogen, nitro, cyano, amino that is unsubstituted or substituted by one or two C1-C6 alkyl or C2-C10 acyl (alkyl-CO—), hydroxyl, unsubstituted or halogenated C1-C6 alkyl, C1-C6 alkoxy, C6-C10 aryl, 3- to 20-membered heteroaryl, C6-C10 aryl-O—, C2-C10 ester group (alkyl-COO—), C2-C10 acyl (alkyl-CO—), C2-C10 acyl-alkoxy (alkyl-OOC—), C2-C10 amido (alkyl NHC(O)—, aryl NHC(O)—), —COOH, hydroxy-C1-C10 alkylene, MeS—, sulfonyl and sulfonamide;
wherein, the two hydrogen atoms on the two adjacent carbon atoms in

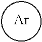

can be replaced by —(CH₂)ₙ— (wherein, n is 1, 2, 3, 4, 5, or 6).

R_{c} is selected from the group consisting of: H, substituted or unsubstituted C1-C6 alkyl, substituted or unsubstituted C1-C6 alkenyl, substituted or unsubstituted C6-C20 aryl, substituted or unsubstituted 3- to 20-membered heteroaryl, substituted or unsubstituted C7-C25 alkyl-aryl, substituted or unsubstituted C1-C5 alkyl-3- to 20-membered heteroaryl, substituted or unsubstituted C3-C20 cycloalkyl, substituted or unsubstituted C1-C5 alkyl-C3-C20 cycloalkyl, substituted or unsubstituted 3- to 20-membered heterocyclyl, or substituted or unsubstituted C1-C5 alkyl-3-to 20-membered heterocyclyl;
There is no particular limitation on the ligand, and the ligand can be any of the ligands mentioned above, preferably L32, L33, L34, L35, L37.

The copper catalyst is preferably: CuBr, CuCl, CuI, CuTc, Cu, Cu₂O, CuCN, CuBr₂, Cu(OTf)₂, or Cu (acac)₂.

The reaction temperature is 120-140° C.

R_{c}SO₂M can be sodium C1-C6 alkyl sulfinate or sodium C6-C10 aryl sulfinate; Preferably, R_{c}SO₂M is selected from sodium phenylsulfinate, sodium methylphenylsulfinate, or sodium methylsulfinate.

Compared with Existing Technologies, the Main Advantages of the Present Invention Include:
1. The present invention provided a class of catalytic systems capable of efficiently carrying out copper-catalyzed coupling reactions of aryl chloride/bromide, wherein in the catalytic systems, the reaction can occur at room temperature, with milder conditions, less ligand dosage, and good substrate compatibility. The catalytic system has a wide range of applications.
2. Compared to the coupling reaction methods of aryl chloride in the prior art, the method of the present invention adopts a lower cost copper catalyst system, mild reaction conditions (ligand and catalyst dosage can be as low as 1.0 mol %, and the temperature can reach 100° C.). The ligand structure is simple and easy to be prepared, the dosage used is further reduced, and the reaction is economical.
3. Compared with the coupling reaction methods of aryl bromide in the prior art, the method of the present invention adopts a low-cost copper catalyst system, and the reaction can occur at room temperature under milder reaction conditions. The ligand is used in an ultra-low amount (0.01 mol %), greatly reducing the cost of the reaction.
4. The raw materials of aryl chlorides/bromides, compared to aryl iodides, used in the catalytic system of the present invention, have the advantages of low raw material cost, wide availability, and commercial accessibility. The efficient catalytic system, on the one hand, reduces the temperature of the reaction, further expanding the applicability of the substrate, on the other hand, reduces the dosage of catalyst and ligand, and further lowers the cost of the reaction. It will have broad application prospects in medicinal chemistry, fine chemical engineering, and the production of bulk chemicals in the future.

The present invention will be further explained in conjunction with specific examples. It should be understood that these examples are only used to illustrate the present invention and not to limit the scope of the present invention. The experimental methods without specific conditions specified in the following examples are usually carried out under conventional conditions or conditions recommended by the manufacturer. Unless otherwise specified, percentages and parts are calculated by weight.

### Example 1: Ligand Preparation Method I

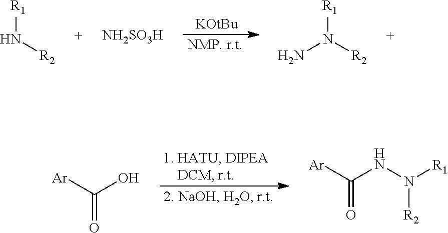

substituted or unsubstituted pyrrole/indole/azoles/benzazole and other nitrogen-containing aromatic heterocycles, etc. (1.0 eq) were placed in a three necked bottle. NMP was added and the mixture was stirred at room temperature. The bottle was plugged with a rubber stopper, inserted with a balloon, and a solution of aminosulfonic acid (1.5 eq) in NMP and a solution of potassium tert butoxide (2.0 eq) in NMP were added simultaneously to the system. After dropwise addition, the reaction was stirred at room temperature for 3 hours, switched to a distillation apparatus, and distilled under reduced pressure with an oil pump. NMP was recovered for recycling. The residue was added with water, and extracted with EA. The organic phases were combined, than washed with saturated sodium chloride and water, dried over anhydrous sodium sulfate, and further pumped dried with oil pump after removing excess solvent by rotary evaporation, which was directly subjected to the next condensation reaction without separation.

The resulting residue of the previous step was transferred to a single necked bottle, added with heterocyclic carboxylic acid (1.0 eq), DIPEA (2.0 eq), HATU (2.0 eq), and DCM (0.2 M), reacted overnight at room temperature, the reaction was filtered through diatomaceous earth, washed with DCM, and spun to remove DCM solvent. H₂O and 5% NaOH were added to adjust the pH of the system to 14; after the aqueous phase was washed with DCM to remove impurities, the aqueous phases were combined and adjusted to pH=6 by adding 2M hydrochloric acid. At this point, solids precipitated in the aqueous phase and the mixture was filtered directly. The filter cake was washed with DCM, EtOH, etc., and dried to obtain the solid (if impure, separate it by column chromatography).

### Example 2: Ligand Preparation Method II

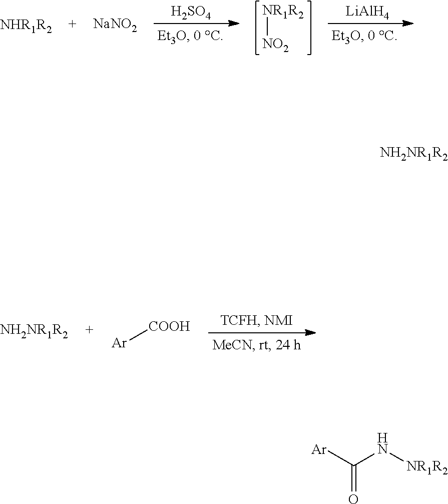

Substituted or unsubstituted pyrrole/indole/azoles/benzazole and other nitrogen-containing aromatic heterocycles as well as di-substituted amine compounds were dispersed in ether and added with 50% sulfuric acid. Sodium nitrite (2.0 eq) was dissolved in water (0.25 volume) and the mixture was dropped into a dispersion of ether-sulfuric acid containing the substrate at 0° C. After about 20 minutes, the mixture was transferred into room temperature, fractioned, and the aqueous phase was extracted with ether. The ether solution was dried over anhydrous sodium sulfate and added with a small amount of sodium bicarbonate to remove acid, then the ether solution was concentrated under low temperature and reduced pressure.

Lithium aluminum hydride (2.0 eq) was taken and added to ether (1.25 volume), the concentrated ether solution was slowly added at 0° C., and maintained for 5 minutes, and then the mixture was transferred into room temperature and reacted for 30-50 minutes. The reaction was quenched with water, added with KOH solution to precipitate metal, and extracted with ethyl acetate. The organic phases were combined, dried over anhydrous sodium sulfate, filtered, and concentrated. The residue was directly subjected to the next condensation reaction without separation.

Aromatic heterocyclic-2-carboxylic acid (1.0 eq.), the residue obtained in the previous step, and tetramethyl chlorouronium hexafluorophosphate (1.2 eq.) were dissolved in acetonitrile. N-methylimidazole (3.5 eq.) was added and the mixture was stirred at room temperature for 24 h. After the reaction was completed, a certain amount of water was added to the system. After stirring for a period of time, precipitates were formed and filtered, the solids were washed with a small amount of methanol or ether, and dried to obtain the corresponding carboxylic acid hydrazide ligand.

### Example 3: Ligand Preparation Method III

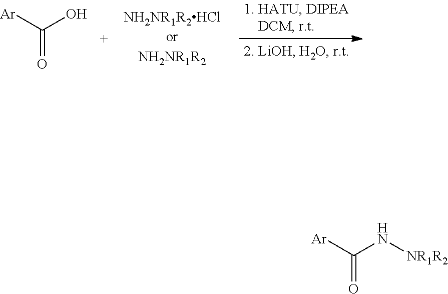

To a bottle was added carboxylic acid (1.0 eq.), HATU (2.0 eq.), DIPEA (2.0 eq., 3.0 eq. required for hydrochloride) and DCM. After stirring for 10 minutes, N-aminopyrrole/indole/azoles/benzazole/other nitrogen-containing aromatic heterocycles or disubstituted hydrazine/disubstituted hydrazine hydrochloride (1.0 eq.) was added. The reaction was reacted at room temperature overnight. The reaction solution was filtered to remove insoluble substances and spun dried to remove the solvent. The residue was added with LiOH aqueous solution (2.0 eq.) to adjust the pH of the solution to 14. The mixture was added with a small amount of water and washed twice with DCM. The aqueous phase was added with 2M hydrochloride to adjust the pH to 6, and a large amount of insoluble substances precipitated. After filtration, the filter cake was washed with cold water and ether, and dried to obtain the target carboxylic acid hydrazide compound (if impure, separate it by column chromatography).

### Example 4: Ligand Preparation Method IV

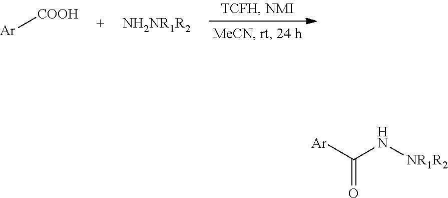

Aromatic heterocyclic-2-carboxylic acid (1.0 eq.), N-aminopyrrole/indole/azoles/benzazole/other nitrogen-containing aromatic heterocycles or disubstituted hydrazine (1.0 eq.) and tetramethyluronium chloride hexafluorophosphate (1.2 eq.) were dissolved in acetonitrile, and N-methylimidazole (3.5 eq.) was added. The mixture was stirred and reacted at room temperature for 24 h. After the reaction was completed, a certain amount of water was added to the system. After stirring for a period of time, precipitates were formed and filtered. The solids were washed with a small amount of methanol or ether, and dried to obtain the corresponding heterocyclic-2-carboxylic acid hydrazide.

### Example 5: Ligand Preparation Method V

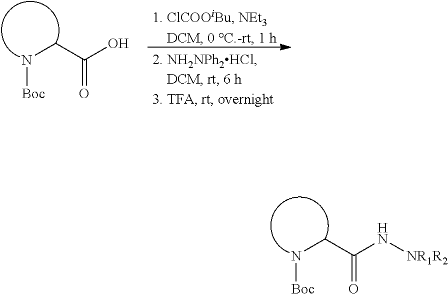

To a bottle was added N-Boc protected carboxylic acid (1.0 eq.), and added with triethylamine (1.3 eq.) and ultra dry DCM using a syringe. Pre-prepared isobutyl chloroformate/DCM solution (1.3 eq.) was dripped slowly under stirring in an ice bath. After addition, the reaction was moved to room temperature and stirred for 1 hour. Pre-prepared solutions of N-aminopyrrole/indole/azoles/benzazole/other nitrogen-containing aromatic heterocycles or disubstituted hydrazine (1.0 eq) and triethylamine (1.2 eq) in DCM was added slowly under stirring and in an ice bath. After addition, the reaction was moved to room temperature and stirred for 6 hour. Excess TFA was added and the reaction continued at room temperature overnight. The reaction solution was evaporated to remove the solvent, then treated with 10% ammonia water until turning alkaline, and filtered. The filter cake was pulped with DCM and separated to obtain the target hydrazide compound.

### Example 6: Ligand Preparation Method VI

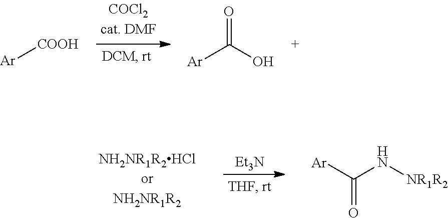

Heterocyclic-2-carboxylic acid (1 eq.) was dissolved in anhydrous dichloromethane, added with 2 drops of DMF, and slowly added with oxalyl chloride (3 eq.) under stirring. A large amount of bubbles were generated in the system. After the addition was complete, the solid in the system gradually dissolved. The reaction was stirred and reacted at room temperature for 2 h. The system was directly concentrated to dryness to obtain heterocyclic carboxylic acid chloride solid, which was directly used in the next reaction.

N-aminopyrrole/indole/azoles/benzazole/other nitrogen-containing aromatic heterocycles or disubstituted hydrazine/disubstituted hydrazine hydrochloride was taken and dissolved in tetrahydrofuran (0.3 M), and triethylamine (3 eq.) was added. The heterocyclic carboxylic acid chloride obtained in the previous step was dissolved in tetrahydrofuran under stirring, and the mixture was added dropwise to the aniline system, and stirred at room temperature for 1 hour. The system produced a large amount of solid, concentrated directly to dryness, diluted with water, and filtered. The filter cake was washed with cold ether or methanol, and dried to obtain the corresponding heterocyclic carboxylic acid hydrazide (if impure, separate it by column chromatography).

| | | Preparation |
|---|---|---|
| Ligand structure | Structural characterization data | method |
| 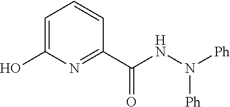 [SMILES: O=C(NN(c1ccccc1)c1ccccc1)c1cccc(O)n1] | ¹H NMR (500 MHz, DMSO-d₆) δ 11.18 (d, J = 10.3 Hz, 2H), 7.71 (t, J = 7.9 Hz, 1H), 7.28 (dd, J = 8.6, 7.2 Hz, 5H), 7.17-7.10 (m, 4H), 7.02-6.94 (m, 2H), 6.78 (d, J = 8.6 Hz, 1H). ¹³C NMR (151 MHz, DMSO- d₆) δ 162.56, 162.40, 145.56, 143.73, 140.25, 129.05, 122.31, 118.92, 117.78, | III |
| L-1, yield 78% | 111.14. HRMS (ESI) m/z calculated for | |
| | C₁₈H₁₅N₃NaO₂⁺ 328.1059 (M + Na)⁺, found | |
| | 328.1056. | |
| 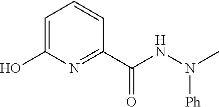 [SMILES: CN(NC(=O)c1cccc(O)n1)c1ccccc1] | ¹H NMR (500 MHz, DMSO-d₆) δ 7.72-7.69 (m, 1H), 7.24 (d, J = 7.0 Hz, 1H), 7.23-7.20 (m, 2H), 6.83 (d, J = 8.0 Hz, 2H), 6.79-6.76 (m, 2H), 3.17 (s, 3H). ¹³C NMR (126 MHz, DMSO-d₆) δ = 162.37, 161.94, 149.63, 143.48, 140.28, 128.88, 118.54, 117.96, 112.54, 110.54, 40.09. | III |
| L-2, yield 80% | | |
| 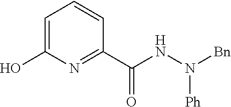 [SMILES: O=C(NN(Cc1ccccc1)c1ccccc1)c1cccc(O)n1] | ¹H NMR (500 MHz, DMSO-d₆) δ 11.22 (brs, 1H), 10.60 (brs, 1H), 7.70 (t, J = 7.7 Hz, 1H), 7.49 (d, J = 7.4 Hz, 2H), 7.35 (t, J = 7.4 Hz, 2H), 7.27 (t, J = 7.3 Hz, 1H), 7.20 (t, J = 8.5 Hz, 3H), 6.83 (d, J = 8.1 Hz, 2H), 6.78 (t, J = 7.2 Hz, 2H), 4.76 (s, 2H). δ¹³C NMR (126 MHz, DMSO-d₆) δ = 162.42, | III |
| L-3, yield 73% | 162.13, 148.84, 140.33, 138.04, 128.94, | |
| | 128.37, 127.70, 127.13, 118.77, 112.76, | |
| | 56.88. | |
| 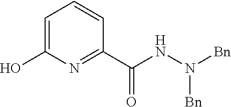 [SMILES: O=C(NN(Cc1ccccc1)Cc1ccccc1)c1cccc(O)n1] | ¹H NMR (500 MHz, DMSO-d₆) δ 9.36 (brs, 1H), 7.60 (t, J = 7.8 Hz, 1H), 7.43 (d, J = 7.6 Hz, 4H), 7.31 (t, J = 7.4 Hz, 4H), 7.23 (t, J = 7.2 Hz, 2H), 7.04 (brs, 1H), 6.68 (d, J = 8.6 Hz, 1H), 4.08 (s, 4H). ¹³C NMR (126 MHz, DMSO-d₆) δ = 162.40, 160.91, 146.35, 140.17, 137.81, 128.63, 128.18, 127.15, | III |
| L-4, yield 77% | 118.59, 110.30, 59.52. | |
| 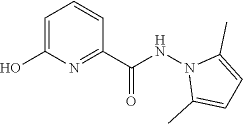 [SMILES: Cc1ccc(C)n1NC(=O)c1cccc(O)n1] | ¹H NMR (500 MHz, DMSO-d₆) δ 11.25 (brs, 1H), 11.23 (brs, 1H), 7.77-7.75 (m, 1H), 7.34 (d, J = 5.3 Hz, 1H), 6.85 (d, J = 7.2 Hz, 1H), 5.69 (s, 2H), 2.01 (s, 6H). ¹³C NMR (151 MHz, DMSO-d₆) δ = 162.92, 162.60, 144.01, 140.29, 127.01, 117.11, 111.96, 103.17. HRMS (ESI) m/z calculated for C₁₂H₁₃N₃NaO₂⁺ 254.0905 (M + Na)⁺, | III |
| L-5, yield 67% | found 254.0900. | |
| 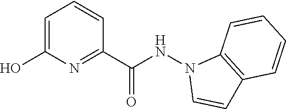 [SMILES: O=C(Nn1ccc2ccccc21)c1cccc(O)n1] | ¹H NMR (500 MHz, DMSO-d₆) δ 7.79 (brs, 1H), 7.68-7.65 (m, 1H), 7.52 (d, J = 7.8 Hz, 1H), 7.34 (d, J = 8.2 Hz, 1H), 7.17 (d, J = 7.0 Hz, 1H), 7.09 (t, J = 7.53 Hz, 1H), 7.00 (t, J = 7.4 Hz, 1H),. 6.64 (d, J = 8.6 Hz, 1H), 6.40 (d, J = 3.1 Hz, 1H). | III |
| L-6, yield 43% | | |
| 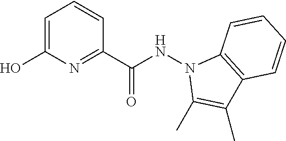 [SMILES: Cc1c(C)n(NC(=O)c2cccc(O)n2)c2ccccc12] | ¹H NMR (500 MHz, DMSO-d₆) δ 11.48 (brs, 2H), 7.80 (t, J = 7.7 Hz, 1H), 7.47 (d, J = 7.4 Hz, 1H), 7.44 (d, J = 6.7 Hz, 1H), 7.15 (d, J = 7.6 Hz, 1H0, 7.10-7.04 (m, 2H), 6.91 (d, J = 8.4 Hz, 1H), 2.23 (s, 3H), 2.20 (s, 3H). ¹³C NMR (126 MHz, DMSO-d₆) δ = 163.56, 162.79, 144.26, 140.42, 135.46, 132.95, 126.51, 121.08, 119.37, 117.76, 117.09, 112.50, 108.26, 104.65, 9.04, 8.72. | I |
| L-7, yield 45% | HRMS (ESI) m/z calculated for | |
| | C₁₆H₁₅N₃NaO₂⁺ 304.1060 (M + Na)⁺, found | |
| | 304.1056. | |
| 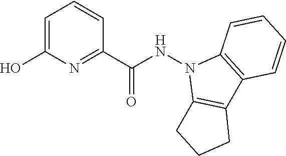 [SMILES: O=C(Nn1c2c(c3ccccc31)CCC2)c1cccc(O)n1] | ¹H NMR (500 MHz, DMSO-d₆) δ 11.52 (brs, 2H), 7.82 (d, J = 7.8 Hz, 1H), 7.45 (d, J = 7.0 Hz, 1H), 7.41 (d, J = 7.1 Hz, 1H), 7.20 (d, J = 7.5 Hz, 1H), 7.11-7.06 (m, 2H), 6.93 (d, J = 8.5 Hz, 1H), 2.85 (t, J = 6.4 Hz, 2H), 2.79 (t, J = 6.7 Hz, 2H), 2.51-2.47 (m, 2H). ¹³C NMR (126 MHz, DMSO-d₆) δ = 163.40, 162.80, 145.58, 144.22, 140.52, 140.41, 122.61, 120.58, 119.75, 118.34, 116.90, 116.31, 112.60, 109.41. HRMS (ESI) m/z | I |
| L-8, yield 40% | calculated for C₁₇H₁₅N₃NaO₂⁺ 316.1059 | |
| | (M + Na)⁺, found 316.1056. | |
| 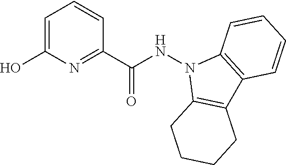 [SMILES: O=C(Nn1c2c(c3ccccc31)CCCC2)c1cccc(O)n1] | ¹H NMR (500 MHz, DMSO-d₆) δ 11.40 (brs, 2H), 7.78 (t, J = 6.4 Hz, 1H), 7.43 (d, J = 7.6 Hz, 1H), 7.40 (d, J = 6.4 Hz, 1H), 7.16 (d, J = 7.9 Hz, 1H), 7.09-7.06 (m, 1H), 7.05- 7.02 (m, 1H), 6.89 (d, J = 8.5 Hz, 1H), 2.66 (t, J = 5.2 Hz, 2H), 2.54-2.50 (m, 2H), 1.84- 1.80 (m, 4H). ¹³C NMR (151 MHz, DMSO- d₆) δ = 163.44, 162.67, 144.25, 140.33, 135.79, 135.72, 125.29, 121.00, 119.32, 117.54, 117.06, 112.31, 108.35, 107.64, | I |
| L-9, yield 52% | 22.75, 22.30, 20.54, 20.45. HRMS (ESI) | |
| | m/z calculated for C₁₈H₁₇N₃NaO₂⁺ 330.1215 | |
| | (M + Na)⁺, found 330.1213. | |
| 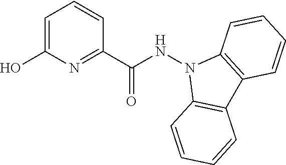 [SMILES: O=C(Nn1c2ccccc2c2ccccc21)c1cccc(O)n1] | ¹H NMR (500 MHz, DMSO-d₆) δ 11.77 (s, 1H), 11.32 (s, 1H), 8.18 (d, J = 7.7 Hz, 2H), 7.82 (s, 1H), 7.43 (ddd, J = 8.1, 7.1, 1.2 Hz, 3H), 7.35 (d, J = 8.1 Hz, 2H), 7.25 (t, J = 7.4 Hz, 2H), 6.90 (s, 1H). ¹³C NMR (151 MHz, DMSO-d₆) δ = 163.82, 163.23, 144.77, 140.89, 140.27, 126.61, 121.26, 120.88, 120.49, 117.36, 113.23, 109.28. HRMS (ESI) m/z calculated for C₁₈H₁₃N₃NaO₂⁺ 326.0902 (M + Na)⁺, found 326.0900. | III |
| L-10, yield 73% | | |
| 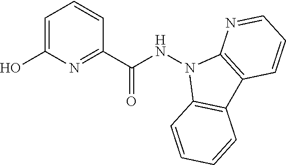 [SMILES: O=C(Nn1c2ccccc2c2cccnc21)c1cccc(O)n1] | ¹H NMR (500 MHz, DMSO-d₆) δ 11.73 (brs, 1H), 11.36 (brs, 1H), 8.61 (dd, J = 1.4, 7.7 Hz, 1H), 8.44 (dd, J = 1.4, 4.8 Hz, 1H), 8.26 (d, J = 7.8 Hz, 1H), 7.9 (t, J = 7.7 Hz, 1H), 7.53 (t, J = 7.6 Hz, 1H), 7.49 (brs, 1H), 7.45 (d, J = 8.1 Hz, 1H), 7.35-7.32 (m, 2H), 6.94 (d, J = 7.9 Hz, 1H). ¹³C NMR (151 MHz, DMSO-d₆) δ = 163.38, 162.75, 150.14, 146.30, 140.43, 139.32, 128.97, 127.24, 121.39, 120.84, 118.42, 116.60, | I |
| L-11, yield 80% | 114.21, 113.26, 109.16. HRMS (ESI) m/z | |
| | calculated for C₁₇H₁₂N₄NaO₂⁺ 327.0849 | |
| | (M + Na)⁺, found 327.0852. | |
| 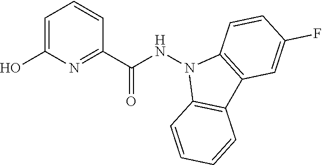 [SMILES: O=C(Nn1c2ccccc2c2cc(F)ccc21)c1cccc(O)n1] | ¹H NMR (500 MHz, DMSO-d₆) δ 11.62 (brs, 2H), 8.21 (d, J = 7.8 Hz, 1H), 8.07 (dd, J = 2.5, 9.2 Hz, 1H), 7.83 (t, J = 6.4 Hz, 1H), 7.49-7.47 (m, 2H), 7.40-7.37 (m, 2H), 7.32-7.26 (m, 2H), 6.93 (d, J = 8.5 Hz, 1H). HRMS (ESI) m/z calculated for C₁₈H₁₂FN₃NaO₂⁺ 344.0804 (M + Na)⁺, found 344.0806. | I |
| L-12, yield 75% | | |
| 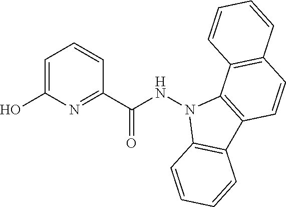 [SMILES: O=C(Nn1c2ccccc2c2ccc3ccccc3c21)c1cccc(O)n1] | ¹H NMR (500 MHz, DMSO-d₆) δ 12.45 (brs, 1H), 11.42 (brs, 1H), 8.71-8.69 (m, 1H), 8.33 (d, J = 8.5 Hz, 1H), 8.28 (d, J = 7.8 Hz, 1H), 8.09-8.08 (m, 1H), 7.90 (t, J = 7.8 Hz, 1H), 7.79 (d, J = 8.5 Hz, 1H), 7.57-7.54 (m, 3H), 7.48 (d, J = 3.86 Hz, 2H), 7.37- 7.34 (m, 1H), 7.02 (d, J = 8.3 Hz, 1H). ¹³C NMR (151 MHz, DMSO-d₆) δ = 163.71, 162.98, 145.10, 140.57, 139.66, 133.56, 132.78, 128.93, 125.74, 125.33, 125.27, 121.19, 121.13, 121.07, 120.95, 120.69, 119.84, 119.22, 116.95, 116.23, 113.69, 109.30. HRMS (ESI) m/z calculated for | I |
| L-13, yield 49% | C₂₂H₁₅N₃NaO₂⁺ 376.1053 (M + Na)⁺, | |
| | found 376.1056. | |
| 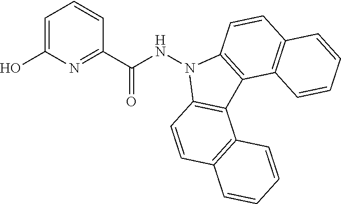 [SMILES: O=C(Nn1c2ccc3ccccc3c2c2c3ccccc3ccc21)c1cccc(O)n1] | ¹H NMR (500 MHz, DMSO-d₆) δ 9.18 (brs, 1H), 9.16 (brs, 1H), 8.15 (d, J = 7.8 Hz, 2H), 8.02 (d, J = 8.6 Hz, 2H), 7.87 (t, J = 7.6 Hz, 1H), 7.80-7.77 (m, 4H), 7.59-7.56 (m, 3H). 6.99 (d, J = 8.4 Hz, 1H). ¹³C NMR (126 MHz, DMSO-d₆) δ = 163.85, 163.01, 144.57, 140.58, 136.80, 130.03, 129.55, 128.40, 127.11, 126.00, 124.42, 123.52, 117.06, 114.72, 112.79, 111.03. HRMS (ESI) m/z calculated for C₂₆H₁₇N₃NaO₂⁺ 426.1215 (M + Na)⁺, found 426.1213. | I |
| L-14, yield 58% | | |
| 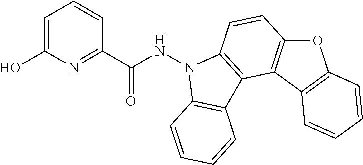 [SMILES: O=C(Nn1c2ccccc2c2c3c(ccc21)oc1ccccc13)c1cccc(O)n1] | ¹H NMR (500 MHz, DMSO-d₆) δ 11.85 (brs, 1H), 11.51 (brs, 1H), 8.38 (d, J = 7.7 Hz, 1H), 8.20 (d, J = 8.5 Hz, 1H), 8.18 (d, J = 7.5 Hz, 1H), 7.87 (d, J = 8.1 Hz, 2H), 7.57-7.42 (m, 7H), 6.96 (d, J = 7.7 Hz, 1H). HRMS (ESI) m/z calculated for C₂₄H₁₅N₃NaO₃⁺ 416.0999 (M + Na)⁺, found 416.1006. | I |
| L-15, yield 55% | | |
| 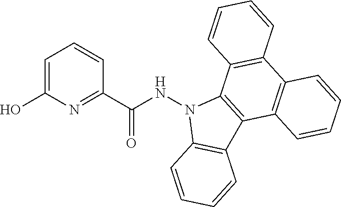 [SMILES: O=C(Nn1c2ccccc2c2c3ccccc3c3ccccc3c21)c1cccc(O)n1] | ¹H NMR (500 MHz, DMSO-d₆) δ 12.55 (brs, 1H), 11.43 (brs, 1H), 9.00 (d, J = 7.99 Hz, 1H), 8.97-8.95 (m, 2H), 8.90 (d, J = 8.12 Hz, 1H), 8.72 (d, J = 8.1 Hz, 1H), 7.91 (t, J = 7.8 Hz, 1H), 7.87-7.84 (m, 1H), 7.73- 7.70 (m, 1H), 7.69-7.66 (m, 2H), 7.57-7.54 (m, 2H), 7.52-7.49 (m, 1H), 7.47-7.44 (m, 1H), 7.04 (d, J = 7.9 Hz, 1H). ¹³C NMR (151 MHz, DMSO-d₆) δ = 163.70, 163.01, 145.43, 140.59, 139.65, 132.61, 130.02, 128.89, 127.93, 127.00, 126.73, 126.61, 124.56, 124.41, 124.10, 123.97, 123.68, 122.38, 121.74, 121.70, 121.65, 121.27, | I |
| L-16, yield 48% | 116.09, 113.74, 111.43, 109.78. HRMS | |
| | (ESI) m/z calculated for C₂₆H₁₇N₃NaO₂⁺ | |
| | 426.1211 (M + Na)⁺, found 426.1213. | |
| 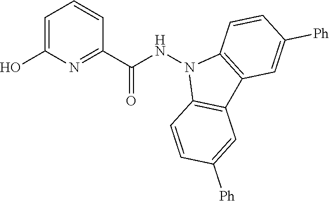 [SMILES: O=C(Nn1c2ccc(-c3ccccc3)cc2c2cc(-c3ccccc3)ccc21)c1cccc(O)n1] | ¹H NMR (500 MHz, DMSO-d₆) δ 11.81 (brs, 1H), 11.55 (brs, 1H), 8.69 (d, J = 1.3 Hz, 2H), 7.87-7.85 (m, 1H), 7.83-7.82 (m, 4H), 7.80 (dd, J = 1.7, 8.5 Hz, 2H), 7.51 (t, J = 7.74 Hz, 5H), 7.47 (d, J = 8.4 Hz, 2H), 7.36 (t, J = 7.4 Hz, 2H), 6.96 (d, J = 8.4 Hz, 1H). ¹³C NMR (151 MHz, DMSO-d₆) δ = 163.43, 162.78, 144.41, 140.88, 140.44, 139.88, 132.66, 128.92, 126.76, 126.70, 125.34, 121.73, 119.01, 116.60, 112.90, 109.39. HRMS (ESI) m/z calculated for C₃₀H₂₁N₃NaO₂⁺ 478.1514 (M + Na)⁺, found 478.1526. | I |
| L-17, yield 63% | | |
| 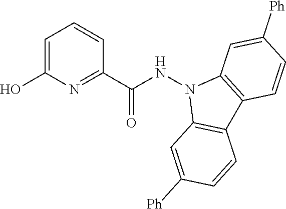 [SMILES: O=C(Nn1c2cc(-c3ccccc3)ccc2c2ccc(-c3ccccc3)cc21)c1cccc(O)n1] | ¹H NMR (500 MHz, DMSO-d₆) δ 11.98 (brs, 1H), 11.47 (brs, 1H), 8.29 (d, J = 8.1 Hz, 2H), 7.82 (t, J = 7.8 Hz, 1H), 7.70 (d, J = 7.3 Hz, 4H), 7.04 (s, 2H), 7.61 (dd, J = 1.5, 8.1 Hz, 2H), 7.49-7.47 (m, 5H), 7.37 (t, J = 7.4 Hz, 2H), 6.94 (d, J = 7.89 Hz, 1H). ¹³C NMR (151 MHz, DMSO-d₆) δ = 163.15, 162.70, 143.54, 141.03, 140.61, 140.37, 138.58, 128.98, 127.37, 127.04, 121.08, 119.99, 119.42, 117.62, 112.45, 106.76. HRMS (ESI) m/z calculated for C₃₀H₂₁N₃NaO₂⁺ 478.1514 (M + Na)⁺, found 478.1526. | I |
| L-18, yield 38% | | |
| 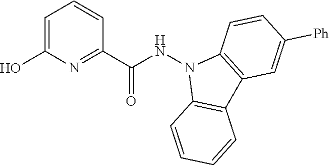 [SMILES: O=C(Nn1c2ccccc2c2cc(-c3ccccc3)ccc21)c1cccc(O)n1] | ¹H NMR (500 MHz, DMSO-d₆) δ 11.87 (brs, 1H), 11.38 (brs, 1H), 8.54 (d, J = 1.5 Hz, 1H), 8.31 (d, J = 7.7 Hz, 1H), 7.86-7.83 (m, 1H), 7.80-7.89 (m, 2H), 7.77 (dd, J = 1.7, 8.5 Hz, 1H), 7.50 (t, J = 7.7 Hz, 3H), 7.46 (t, J = 8.0 Hz, 2H), 7.40 (d, J = 8.1 Hz, 1H), 7.36 (t, J = 7.4 Hz, 1H), 7.31-7.29 (m, 1H), 6.95 (d, J = 8.5 Hz, 1H). ¹³C NMR (151 MHz, DMSO-d₆) δ = 163.39, 162.76, 144.34, 140.97, 140.43, 140.31, 139.38, | I |
| L-19, yield 45% | 132.62, 128.91, 126.82, 126.66, 126.37, | |
| | 125.22, 121.49, 120.99, 120.72, 120.17, | |
| | 118.65, 116.96, 112.74, 109.26, 108.95. | |
| | HRMS (ESI) m/z calculated for | |
| | C₂₄H₁₇N₃NaO₂⁺ 402.1212 (M + Na)⁺, | |
| | found 402.1213. | |
| 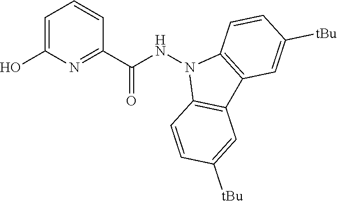 [SMILES: CC(C)(C)c1ccc2c(c1)c1cc(C(C)(C)C)ccc1n2NC(=O)c1cccc(O)n1] | ¹H NMR (500 MHz, DMSO-d₆) δ 11.58 (brs, 2H), 8.27 (s, 2H), 7.82 (t, J = 7.6 Hz, 1H), 7.50-7.45 (m, 3H), 7.26 (d, J = 8.4 Hz, 2H), 6.93 (d, J = 8.2 Hz, 1H), 1.43 (s, 18H). ¹³C NMR (126 MHz, DMSO-d₆) δ = 163.28, 162.77, 142.44, 140.42, 138.50, 123.55, 120.95, 116.68, 116.63, 112.47, 108.25, 34.55, 31.93. HRMS (ESI) m/z calculated for C₂₆H₂₉N₃NaO₂⁺ 438.2153 (M + Na)⁺, found 428.2152. | I |
| L-20, yield 54% | | |
| 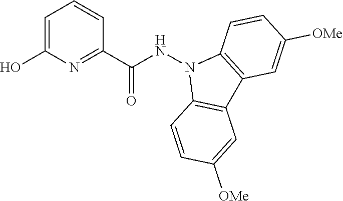 [SMILES: COc1ccc2c(c1)c1cc(OC)ccc1n2NC(=O)c1cccc(O)n1] | ¹H NMR (500 MHz, DMSO-d₆) δ 11.58 (s, 1H), 8.38-8.35 (m, 1H), 8.12 (dd, J = 0.48, 7.5 Hz, 1H), 7.81 (d, J = 2.4 Hz, 2H), 7.76 (d, J = 8.3 Hz, 1H), 7.28 (d, J = 8.8 Hz, 2H), 7.06 (dd, J = 2.5, 8.8 Hz, 2H), 3.88 (s, 6H). ¹³C NMR (126 MHz, DMSO-d₆) δ 161.69, 158.40, 156.66, 153.33, 146.51, 143.18, 134.68, 120.73, 115.55, 114.61, 109.22, 102.90, 55.07. HRMS (ESI) m/z calculated for C₂₀H₁₇N₃NaO₄⁺ 386.1110 (M + Na)⁺, found 386.1111. | I |
| L-21, yield 33% | | |
| 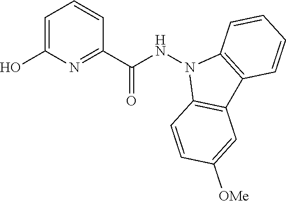 [SMILES: COc1ccc2c(c1)c1ccccc1n2NC(=O)c1cccc(O)n1] | ¹H NMR (500 MHz, DMSO-d₆) δ 11.76 (brs, 2H), 8.08-8.05 (m, 2H), 7.82-7.80 (m, 1H), 7.46 (d, J = 6.9 Hz, 1H), 7.35 (t, J = 7.5 Hz, 1H), 7.29 (d, J = 8.0 Hz, 1H), 7.22 (t, J = 7.4 Hz, 1H), 6.93 (d, J = 8.6 Hz, 1H), 6.88-6.86 (m, 2H), 3.82 (s, 3H). ¹³C NMR (151 MHz, DMSO-d₆) δ = 163.21, 162.92, 159.08, 144.06, 141.37, 140.33, 139.81, 124.67, 121.30, 121.02, 120.03, 119.48, 117.30, 114.40, 112.29, 108.80, 108.60, 92.76, 55.50. HRMS (ESI) m/z calculated for C₁₉H₁₅N₃NaO₃⁺ 356.1003 (M + Na)⁺, found 356.1006. | I |
| L-22, yield 49% | | |
| 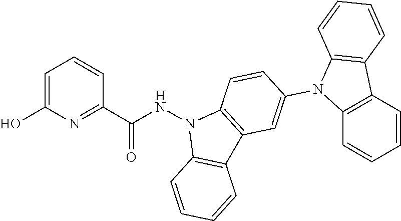 [SMILES: O=C(Nn1c2ccccc2c2cc(-n3c4ccccc4c4ccccc43)ccc21)c1cccc(O)n1] | ¹H NMR (500 MHz, DMSO-d₆) δ 11.94 (brs, 1H), 11.39 (brs, 1H), 8.50 (t, J = 1.1 Hz, 1H), 8.29 (t, J = 7.0 Hz, 3H), 8.16 (brs, 1H), 7.87 (brs, 1H), 7.63 (brs, 2H), 7.52 (t, J = 7.3 Hz, 1H), 7.45-7.42 (m, 3H), 7.35 (d, J = 8.1 Hz, 2H), 7.30 (t, J = 7.4 Hz, 3H), 6.96 (brs, 1H). HRMS (ESI) m/z calculated for C₃₀H₂₀N₄NaO₂⁺ 491.1470 (M + Na)⁺, found 491.1478. | I |
| L-23, yield 66% | | |
| 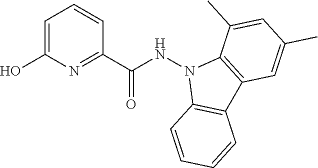 [SMILES: Cc1cc(C)c2c(c1)c1ccccc1n2NC(=O)c1cccc(O)n1] | ¹H NMR (500 MHz, DMSO-d₆) δ 11.68 (brsm 2H), 8.10 (d, J = 7.7 Hz, 1H), 7.83- 7.79 (m, 2H), 7.42-7.38 (m, 2H), 7.30 (d, J = 8.1 Hz, 1H), 7.22 (t, J = 7.42, 1H), 7.00 (s, 1H), 6.91 (d, J = 8.4 Hz, 1H), 2.49 (s, 3H), 2.43 (s, 3H). HRMS (ESI) m/z calculated for C₂₀H₁₇N₃NaO₂⁺ 354.1216 (M + Na)⁺, found 354.1213. | I |
| L-24, yield 32% | | |
| 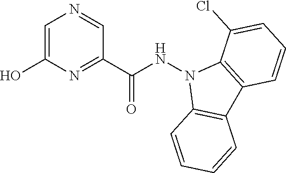 [SMILES: O=C(Nn1c2ccccc2c2cccc(Cl)c21)c1cncc(O)n1] | ¹H NMR (500 MHz, DMSO-d₆) δ 11.92 (brs, 1H), 11.37 (brs, 1H), 8.26-8.23 (m, 2H), 7.82 (t, J = 7.4 Hz, 1H), 7.62 (d, J = 7.6 Hz, 1H), 7.50 (t, J = 7.4 Hz, 1H), 7.46-7.40 (m, 2H), 7.33 (t, J = 7.4 Hz, 1H), 7.20 (t, J = 7.7 Hz, 1H), 6.90 (brs, 1H). | I |
| L-25, yield 22% | | |
| 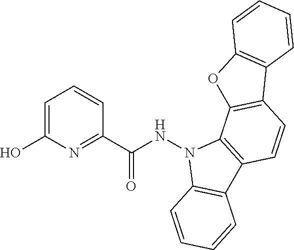 [SMILES: O=C(Nn1c2ccccc2c2ccc3c4ccccc4oc3c21)c1cccc(O)n1] | ¹H NMR (500 MHz, DMSO-d₆) δ 12.20 (brs, 1H), 11.45 (brs, 1H), 8.30 (d, J = 7.8 Hz, 1H), 8.25-8.21 (m, 2H), 8.01 (d, J = 8.2 Hz, 1H), 7.91-7.88 (m, 1H), 7.62 (d, J = 7.6 Hz, 1H), 7.50-7.42 (m, 5H), 7.35 (t, J = 7.8 Hz, 1H), 6.98 (brs, 1H). HRMS (ESI) m/z calculated for C₂₄H₁₅N₃NaO₃⁺ 416.0999 (M + Na)⁺, found 416.1006. | I |
| L-26, yield 34% | | |
| 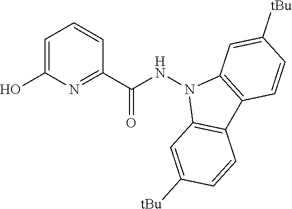 [SMILES: CC(C)(C)c1ccc2c3ccc(C(C)(C)C)cc3n(NC(=O)c3cccc(O)n3)c2c1] | ¹H NMR (500 MHz, DMSO-d₆) δ 11.71 (brs, 1H), 11.39 (brs, 1H), 8.03 (d, J = 8.2 Hz, 2H), 7.84 (t, J = 6.7 Hz, 1H), 7.47 (brs, 1H), 7.32 (dd, J = 1.4, 8.3 Hz, 2H), 7.24 (s, 2H), 6.93 (brs, 1H), 1.34 (s, 18H). HRMS (ESI) m/z calculated for C₂₆H₂₉N₃NaO₂⁺ 438.2153 (M + Na)⁺, found 428.2152. | I |
| L-27, yield 47% | | |
| 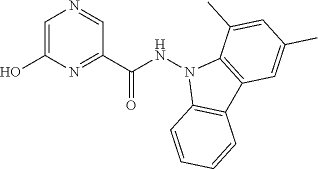 [SMILES: Cc1cc(C)c2c(c1)c1ccccc1n2NC(=O)c1cncc(O)n1] | ¹H NMR (500 MHz, DMSO-d₆) δ 11.82 (brs, 2H), 8.31 (s, 1H), 8.18 (s, 1H), 8.10 (d, J = 7.7 Hz, 1H), 7.81 (s, 1H), 7.39 (t, J = 7.5 Hz, 1H), 7.26 (d, J = 8.1 Hz, 1H), 7.21 (t, J = 7.4 Hz, 1H), 7.00 (s, 1H). | I |
| L-28, yield 21% | | |
| 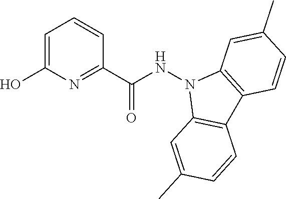 [SMILES: Cc1ccc2c3ccc(C)cc3n(NC(=O)c3cccc(O)n3)c2c1] | ¹H NMR (500 MHz, DMSO-d₆) δ 11.71 (brs, 1H), 11.36 (brs, 1H), 7.98 (d, J = 7.9 Hz, 2H), 7.83 (t, J = 7.8 Hz, 1H), 7.46 (brs, 1H), 7.13 (s, 2H), 7.06 (d, J = 8.0 Hz, 2H), 6.95-6.91 (m, 1H), 2.45 (s, 6H). HRMS (ESI) m/z calculated for C₂₀H₁₇N₃NaO₂⁺ 354.1216 (M + Na)⁺, found 354.1213. | I |
| L-29, yield 55% | | |
| 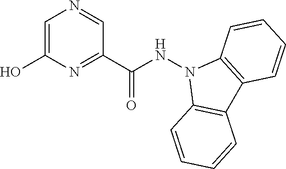 [SMILES: O=C(Nn1c2ccccc2c2ccccc21)c1cncc(O)n1] | ¹H NMR (500 MHz, DMSO-d₆) δ 12.34 (s, 1H), 11.95 (s, 1H), 8.70 (s, 1H), 8.45 (s, 1H), 8.20 (dt, J = 7.7, 0.9 Hz, 2H), 7.46 (ddd, J = 8.2, 7.1, 1.2 Hz, 2H), 7.40 (d, J = 8.1 Hz, 2H), 7.28 (ddd, J = 8.0, 7.0, 1.1 Hz, 2H). ¹³C NMR (500 MHz, DMSO-d₆) δ 162.88, 158.99, 139.75, 126.20, 120.83, 120.46, 120.10, 108.85. HRMS (ESI) m/z calculated for C₁₇H₁₂N₄NaO₂⁺ 327.0849 (M + Na)⁺, found 327.0852. | I |
| L-30, yield 70% | | |
| 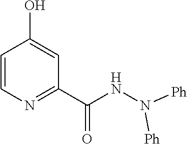 [SMILES: O=C(NN(c1ccccc1)c1ccccc1)c1cc(O)ccn1] | ¹H NMR (500 MHz, DMSO-d₆) δ 11.38 (s, 1H), 11.10 (s, 1H), 8.37 (d, J = 5.5 Hz, 1H), 7.40 (d, J = 2.5 Hz, 1H), 7.26 (t, J = 7.8 Hz, 4H), 7.11 (d, J = 7.2 Hz, 4H), 7.00-6.92 (m, 3H). ¹³C NMR (126 MHz, DMSO-d₆) δ 165.56, 164.21, 151.60, 150.59, 146.13, 129.41, 122.48, 119.32, 114.76, 110.54. HRMS (ESI) m/z calcd. for C₁₈H₁₆N₃O₂ [M + H]⁺ 306.1237 found 306.1246. | III |
| L-31, yield 53% | | |
| 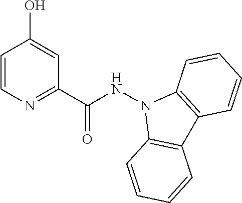 [SMILES: O=C(Nn1c2ccccc2c2ccccc21)c1cc(O)ccn1] | ¹H NMR (500 MHz, DMSO-d₆) δ 12.05 (s, 1H), 11.22 (s, 1H), 8.50 (d, J = 5.6 Hz, 1H), 8.18 (d, J = 7.8 Hz, 2H), 7.51 (d, J = 2.5 Hz, 1H), 7.42 (t, J = 7.6 Hz, 2H), 7.30 (d, J = 8.1 Hz, 2H), 7.25 (t, J = 7.4 Hz, 2H), 7.09 (dd, J = 5.6, 2.5 Hz, 1H). ¹³C NMR (126 MHz, dmso) δ 165.70, 164.52, 150.89, 150.83, 140.25, 126.54, 121.17, 120.82, 120.32, 115.19, 110.92, 109.20. HRMS (ESI) m/z calcd. for C₁₈H₁₄N₃O₂ [M + H]⁺ 304.1081 found 304.1088. | I |
| L-32, yield 66% | | |
| 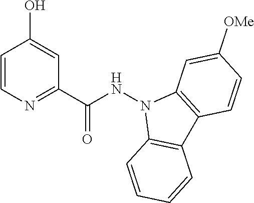 [SMILES: COc1ccc2c3ccccc3n(NC(=O)c3cc(O)ccn3)c2c1] | ¹H NMR (500 MHz, DMSO-d₆) δ 11.99 (s, 1H), 11.20 (s, 1H), 8.50 (d, J = 5.6 Hz, 1H), 8.06 (t, J = 8.0 Hz, 2H), 7.50 (d, J = 2.6 Hz, 1H), 7.33 (t, J = 7.6 Hz, 1H), 7.21 (t, J = 8.2 Hz, 2H), 7.08 (dd, J = 5.6, 2.5 Hz, 1H), 6.86 (dd, J = 8.5, 2.2 Hz, 1H), 6.78 (d, J = 2.3 Hz, 1H), 3.80 (s, 3H). ¹³C NMR (500 MHz, DMSO) δ 165.67, 164.42, 159.47, 150.90, 150.81, 141.82, 140.26, 125.09, 121.74, 121.40, 120.37, 119.91, 115.15, 114.78, 110.92, 109.17, 108.96, 93.01, 55.91. HRMS (ESI) m/z calcd. for C₁₉H₁₆N₃O₃ [M + H]⁺ 334.1186 found 334.1193. | I |
| L-33, yield 56% | | |
| 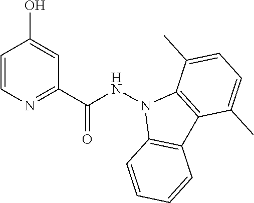 [SMILES: Cc1ccc(C)c2c1c1ccccc1n2NC(=O)c1cc(O)ccn1] | ¹H NMR (500 MHz, DMSO-d₆) δ 12.16 (s, 1H), 11.19 (s, 1H), 8.51 (d, J = 5.6 Hz, 1H), 8.16 (d, J = 7.8 Hz, 1H), 7.50 (d, J = 2.5 Hz, 1H), 7.42 (t, J = 7.6 Hz, 1H), 7.35-7.20 (m, 2H), 7.14-7.01 (m, 2H), 6.94 (d, J = 7.3 Hz, 1H), 2.80 (s, 3H), 2.48 (s, 3H). ¹³C NMR (126 MHz, dmso) δ 165.22, 164.27, 150.48, 140.36, 138.08, 130.38, 128.24, 125.35, 122.12, 121.35, 121.31, 119.99, 119.72, 117.53, 114.64, 110.49, 108.48, 20.33, 17.14. HRMS (ESI) m/z calcd. for C₂₀H₁₈N₃O₂ [M + H]⁺ 332.1394 found 332.1401. | I |
| L-34, yield 21% | | |
| 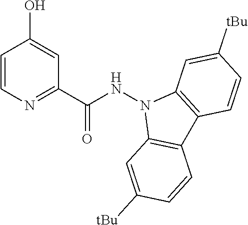 [SMILES: CC(C)(C)c1ccc2c3ccc(C(C)(C)C)cc3n(NC(=O)c3cc(O)ccn3)c2c1] | ¹H NMR (500 MHz, DMSO-d₆) δ 11.91 (s, 1H), 11.17 (s, 1H), 8.50 (d, J = 5.6 Hz, 1H), 8.01 (d, J = 8.2 Hz, 2H), 7.50 (d, J = 2.5 Hz, 1H), 7.30 (d, J = 8.2 Hz, 2H), 7.18 (s, 2H), 7.11-7.06 (m, 1H), 1.32 (s, 18H). ¹³C NMR (500 MHz, DMSO) δ 165.72, 164.46, 150.98, 150.88, 149.12, 140.59, 120.13, 118.75, 118.17, 115.17, 110.94, 105.03, 35.25, 31.99. For C₂₆H₃₀N₃O₂ (M + 1)⁺ requires 416.2343; found: 416.2333. | II |
| L-35, yield 20% | | |
| 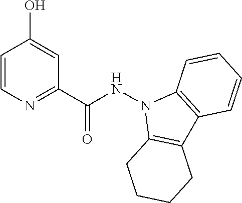 [SMILES: O=C(Nn1c2c(c3ccccc31)CCCC2)c1cc(O)ccn1] | ¹H NMR (500 MHz, DMSO-d₆) δ 11.84 (s, 1H), 11.17 (s, 1H), 8.44 (d, J = 5.6 Hz, 1H), 7.47 (d, J = 2.5 Hz, 1H), 7.39-7.34 (m, 1H), 7.14-6.96 (m, 4H), 2.81 (dd, J = 8.6, 5.4 Hz, 2H), 2.73 (t, J = 7.1 Hz, 2H), 2.69 (s, 2H), 2.50-2.42 (m, 2H). ¹³C NMR (500 MHz, DMSO-d₆) δ 165.21, 163.97, 150.41, 150.32, 145.59, 140.47, 122.54, 120.46, 119.58, 118.25, 116.03, 114.63, 110.39, 109.36, 38.28, 27.58, 24.58, 24.40. HRMS (ESI) m/z calculated for C₁₈H₁₇N₃NaO₂⁺ 330.1215 (M + Na)⁺, found 330.1213. | III |
| L-36, yield 56% | | |
| 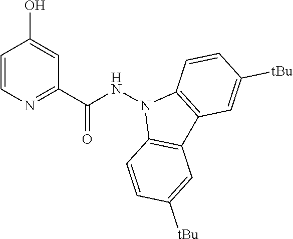 [SMILES: CC(C)(C)c1ccc2c(c1)c1cc(C(C)(C)C)ccc1n2NC(=O)c1cc(O)ccn1] | ¹H NMR (500 MHz, DMSO-d₆) δ 11.96 (s, 1H), 11.19 (s, 1H), 8.49 (d, J = 5.5 Hz, 1H), 8.23 (s, 2H), 7.50-7.42 (m, 3H), 7.17 (d, J = 8.5 Hz, 2H), 7.07 (s, 1H), 1.40 (s, 18H). ¹³C NMR (500 MHz, DMSO) δ 165.69, 164.46, 150.99, 150.79, 142.69, 138.90, 123.92, 121.26, 116.98, 115.12, 110.84, 108.60, 34.95, 32.34. HRMS (ESI) m/z calculated for C₂₆H₂₉N₃NaO₂⁺ 438.2153 (M + Na)⁺, found 428.2152. | I |
| L-37, yield 33% | | |
| 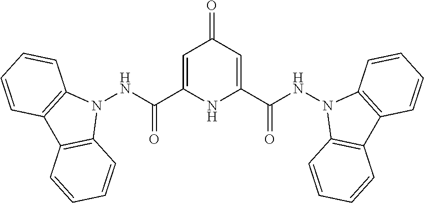 [SMILES: O=C(Nn1c2ccccc2c2ccccc21)c1cc(=O)cc(C(=O)Nn2c3ccccc3c3ccccc32)[nH]1] | ¹H NMR (500 MHz, DMSO-d₆) δ 12.39 (s, 2H), 12.00 (s, 1H), 8.21 (dt, J = 7.8, 1.0 Hz, 4H), 7.75 (s, 2H), 7.52-7.45 (m, 8H), 7.29 (ddd, J = 8.0, 5.9, 2.3 Hz, 4H). ¹³C NMR (500 MHz, DMSO-d₆) δ 162.87, 149.32, 139.80, 126.41, 120.86, 120.62, 120.35, 113.08, 108.93. HRMS (ESI) m/z calcd. for C₃₁H₂₂N₅O₃ [M + H]⁺ 512.1717, found 512.1724. | IV |
| L-38, yield 75% | | |
| 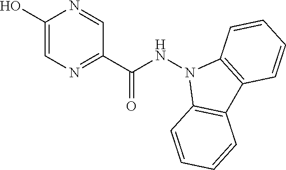 [SMILES: O=C(Nn1c2ccccc2c2ccccc21)c1cnc(O)cn1] | ¹H NMR (500 MHz, DMSO-d₆) δ 13.01 (s, 1H), 11.77 (s, 1H), 8.19 (d, J = 7.7 Hz, 2H), 8.16-8.12 (m, 2H), 7.48-7.41 (m, 2H), 7.32 (d, J = 8.1 Hz, 2H), 7.26 (t, J = 7.4 Hz, 2H). ¹³C NMR (500 MHz, DMSO-d₆) δ 162.69, 156.36, 147.13, 139.96, 131.47, 126.11, 125.05, 120.73, 120.42, 119.91, 108.79. HRMS (ESI) m/z calculated for C₁₇H₁₂N₄NaO₂⁺ 327.0849 (M + Na)⁺, found 327.0852. HRMS (ESI) m/z calcd. for C₁₇H₁₃N₄O₂ [M + H]⁺ 305.1033, found | I |
| L-39, Yield 54% | 305.1041. | |
| 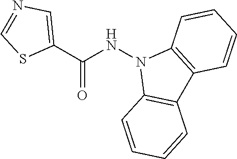 [SMILES: O=C(Nn1c2ccccc2c2ccccc21)c1cncs1] | ¹H NMR (500 MHz, DMSO-d₆) δ 12.12 (s, 1H), 9.45 (s, 1H), 8.84 (s, 1H), 8.21 (d, J = 7.8 Hz, 2H), 7.51-7.43 (m, 4H), 7.29 (ddd, J = 7.9, 5.9, 2.2 Hz, 2H). ¹³C NMR (500 MHz, DMSO-d₆) δ 160.02, 159.54, 145.52, 139.83, 132.11, 126.38, 120.80, 120.57, 120.34, 108.85. HRMS (ESI) m/z calcd. for C₁₆H₁₂N₃OS [M + H]⁺ 294.0696, found 294.0704. | III |
| L-40, yield 59% | | |
| 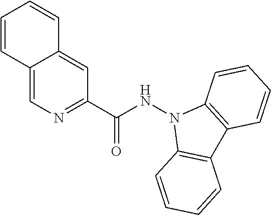 [SMILES: O=C(Nn1c2ccccc2c2ccccc21)c1cc2ccccc2cn1] | ¹H NMR (500 MHz, DMSO-d₆) δ 12.30 (s, 1H), 8.71-8.66 (m, 1H), 8.28 (dd, J = 8.5, 1.1 Hz, 1H), 8.22 (ddd, J = 8.4, 2.8, 1.9 Hz, 3H), 8.18 (dd, J = 8.3, 1.4 Hz, 1H), 7.97 (ddd, J = 8.4, 6.9, 1.5 Hz, 1H), 7.82 (ddd, J = 8.2, 6.9, 1.2 Hz, 1H), 7.45 (ddd, J = 8.1, 6.9, 1.2 Hz, 2H), 7.43-7.37 (m, 2H), 7.28 (ddd, J = 7.9, 6.9, 1.2 Hz, 2H). ¹³C NMR (500 MHz, DMSO-d₆) δ 164.22, 148.87, 146.24, 139.87, 138.18, 130.87, 129.48, 129.28, 128.68, 128.30, 126.15, 120.81, 120.43, 119.99, 119.17, 108.90. HRMS (ESI) m/z calcd. for C₂₂H₁₆N₃O [M + H]⁺ | III |
| L-41, yield 70% | 338.1288, found 338.1293. | |
| 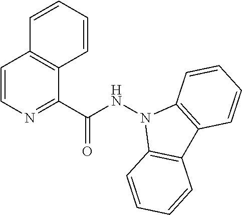 [SMILES: O=C(Nn1c2ccccc2c2ccccc21)c1nccc2ccccc12] | ¹H NMR (500 MHz, DMSO-d₆) δ 12.22 (s, 1H), 8.85 (d, J = 8.6 Hz, 1H), 8.77 (d, J = 5.5 Hz, 1H), 8.24 (d, J = 7.8 Hz, 2H), 8.21 (d, J = 5.6 Hz, 1H), 8.16 (d, J = 8.3 Hz, 1H), 7.90 (t, J = 7.5 Hz, 1H), 7.82 (t, J = 7.7 Hz, 1H), 7.54-7.46 (m, 4H), 7.34-7.28 (m, 2H). ¹³C NMR (500 MHz, DMSO-d₆) δ 166.04, 149.33, 141.26, 139.86, 136.65, 131.10, 129.17, 127.49, 126.34, 125.90, 125.82, 124.47, 120.85, 120.56, 120.11, 108.71. HRMS (ESI) m/z calcd. for C₂₂H₁₆N₃O [M + H]⁺ 338.1288, found 338.1293. | IV |
| L-42, yield 95% | | |
| 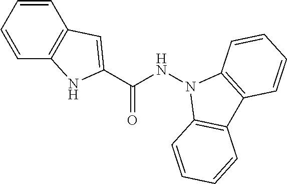 [SMILES: O=C(Nn1c2ccccc2c2ccccc21)c1cc2ccccc2[nH]1] | ¹H NMR (500 MHz, DMSO-d₆) δ 11.93 (s, 1H), 11.87 (s, 1H), 8.23 (d, J = 7.7 Hz, 2H), 7.76 (d, J = 8.0 Hz, 1H), 7.57-7.44 (m, 4H), 7.42 (d, J = 8.1 Hz, 2H), 7.33-7.25 (m, 3H), 7.17-7.10 (m, 1H). ¹³C NMR (500 MHz, DMSO-d₆) δ 161.07, 140.02, 137.07, 128.55, 126.99, 126.32, 124.29, 122.00, 120.73, 120.55, 120.27, 120.15, 112.56, 108.71, 104.57. HRMS (ESI) m/z calcd. for C₂₁H₁₆N₃O [M + H]⁺ 326.1288, found 326.1292. | III |
| L-43, yield 62% | | |
| 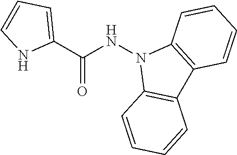 [SMILES: O=C(Nn1c2ccccc2c2ccccc21)c1ccc[nH]1] | ¹H NMR (500 MHz, DMSO-d₆) δ 11.86 (s, 1H), 11.36 (s, 1H), 8.20 (d, J = 7.7 Hz, 2H), 7.46 (ddd, J = 8.2, 7.1, 1.2 Hz, 2H), 7.36 (d, J = 8.1 Hz, 2H), 7.30-7.24 (m, 2H), 7.20 (d, J = 3.8 Hz, 1H), 7.07 (d, J = 3.2 Hz, 1H), 6.29-6.25 (m, 1H). ¹³C NMR (500 MHz, DMSO-d₆) δ 160.29, 140.20, 126.20, 123.37, 123.21, 120.62, 120.47, 119.96, 112.03, 109.29, 108.69. | II |
| L-44, yield 70% | | |
| 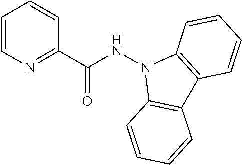 [SMILES: O=C(Nn1c2ccccc2c2ccccc21)c1ccccn1] | ¹H NMR (500 MHz, DMSO-d₆) δ 12.20 (s, 1H), 8.84 (ddd, J = 4.8, 1.7, 1.0 Hz, 1H), 8.22 (dt, J = 7.8, 1.0 Hz, 2H), 8.16 (dt, J = 7.8, 1.2 Hz, 1H), 8.11 (td, J = 7.6, 1.7 Hz, 1H), 7.76 (ddd, J = 7.6, 4.8, 1.4 Hz, 1H), 7.45 (ddd, J = 8.2, 7.0, 1.2 Hz, 2H), 7.36 (dt, J = 8.1, 0.9 Hz, 2H), 7.28 (ddd, J = 8.0, 7.1, 1.0 Hz, 2H). ¹³C NMR (500 MHz, DMSO- d₆) δ 164.07, 148.99, 148.76, 139.85, 138.11, 127.65, 126.14, 122.96, 120.79, | II |
| L-45, yield 69% | 120.43, 119.94, 108.80. HRMS (ESI) m/z | |
| | calcd. for C₁₈H₁₄N₃O [M + H]⁺ 288.1131, | |
| | found 288.1137. | |
| 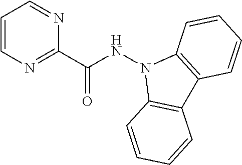 [SMILES: O=C(Nn1c2ccccc2c2ccccc21)c1ncccn1] | ¹H NMR (500 MHz, DMSO-d₆) δ 12.27 (s, 1H), 9.12 (d, J = 4.9 Hz, 2H), 8.21 (dt, J = 7.8, 0.9 Hz, 2H), 7.85 (t, J = 4.9 Hz, 1H), 7.46 (ddd, J = 8.2, 7.0, 1.1 Hz, 2H), 7.38 (dt, J = 8.2, 1.0 Hz, 2H), 7.28 (ddd, J = 8.0, 7.1, 1.1 Hz, 2H). ¹³C NMR (500 MHz, DMSO- d₆) δ 162.39, 158.12, 156.93, 139.68, 126.20, 124.01, 120.80, 120.48, 120.05, 108.81. HRMS (ESI) m/z calcd. for C₁₇H₁₃N₄O [M + H]⁺ 289.1084, found | III |
| L-46, yield 86% | 289.1090. | |
| 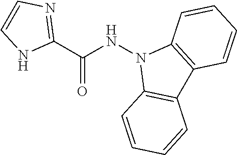 [SMILES: O=C(Nn1c2ccccc2c2ccccc21)c1ncc[nH]1] | ¹H NMR (500 MHz, DMSO-d₆) δ 13.41 (s, 1H), 11.91 (s, 1H), 8.20 (dt, J = 7.7, 0.9 Hz, 2H), 7.49-7.41 (m, 3H), 7.33 (d, J = 8.1 Hz, 2H), 7.30-7.23 (m, 3H). ¹³C NMR (500 MHz, DMSO-d₆) δ 158.18, 139.83, 138.80, 129.63, 126.18, 121.05, 120.66, 120.40, 119.94, 108.70. HRMS [ESI] m/z calcd. for [C₁₆H₁₂N₄O + Na]⁺ 299.0903, found 299.0900. | II |
| L-47, yield 67% | | |
| 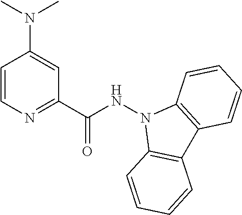 [SMILES: CN(C)c1ccnc(C(=O)Nn2c3ccccc3c3ccccc32)c1] | ¹H NMR (500 MHz, DMSO-d₆) δ 11.96 (s, 1H), 8.33 (d, J = 5.8 Hz, 1H), 8.20 (d, J = 7.7 Hz, 2H), 7.44 (t, J = 7.7 Hz, 2H), 7.33- 7.20 (m, 5H), 6.89 (dd, J = 6.1, 2.9 Hz, 1H), 3.05 (s, 6H). ¹³C NMR (500 MHz, DMSO- d₆) δ 164.84, 154.90, 148.95, 148.72, 139.85, 126.07, 120.71, 120.38, 119.82, 109.30, 108.73, 105.29, 38.88. HRMS (ESI) m/z calcd. for C₂₀H₁₉N₄O [M + H]⁺ 331.1553, found 331.1557. | IV |
| L-48, yield 83% | | |
| 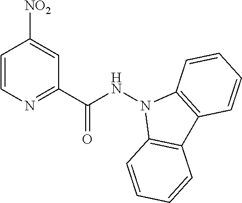 [SMILES: O=C(Nn1c2ccccc2c2ccccc21)c1cc([N+](=O)[O-])ccn1] | ¹H NMR (500 MHz, DMSO-d₆) δ 12.44 (s, 1H), 9.21 (d, J = 5.3 Hz, 1H), 8.62 (d, J = 2.2 Hz, 1H), 8.49 (dd, J = 5.3, 2.3 Hz, 1H), 8.22 (d, J = 7.8 Hz, 2H), 7.49-7.42 (m, 2H), 7.39 (d, J = 8.1 Hz, 2H), 7.29 (t, J = 7.4 Hz, 2H). ¹³C NMR (500 MHz, DMSO-d₆) δ 162.58, 154.98, 151.99, 151.37, 139.67, 126.21, 120.84, 120.49, 120.12, 120.04, 115.57, 108.85. HRMS [ESI] m/z calcd. for [C₁₈H₁₂N₄O₃ + Na]⁺ 355.0802, found 355.0806. | IV |
| L-49, yield 91% | | |
| 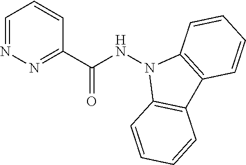 [SMILES: O=C(Nn1c2ccccc2c2ccccc21)c1cccnn1] | ¹H NMR (500 MHz, DMSO-d₆) δ 12.64 (s, 1H), 9.57 (dd, J = 5.1, 1.7 Hz, 1H), 8.32 (dd, J = 8.4, 1.7 Hz, 1H), 8.22 (d, J = 7.8 Hz, 2H), 8.03 (dd, J = 8.4, 5.1 Hz, 1H), 7.49- 7.39 (m, 4H), 7.28 (ddd, J = 8.0, 6.8, 1.3 Hz, 2H). ¹³C NMR (500 MHz, DMSO-d₆) δ 163.02, 154.09, 152.29, 139.71, 128.72, 126.43, 126.20, 120.81, 120.45, 120.07, 108.89. HRMS (ESI) m/z calcd. for C₁₇H₁₃N₄O [M + H]⁺ 298.1084, found 298.1091. | IV |
| L-50, yield 88% | | |
| 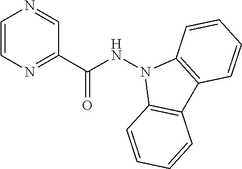 [SMILES: O=C(Nn1c2ccccc2c2ccccc21)c1cnccn1] | ¹H NMR (500 MHz, DMSO-d₆) δ 12.31 (s, 1H), 9.30 (d, J = 1.5 Hz, 1H), 9.03 (d, J = 2.5 Hz, 1H), 8.91 (dd, J = 2.5, 1.5 Hz, 1H), 8.21 (dt, J = 7.8, 1.0 Hz, 2H), 7.45 (ddd, J = 8.1, 7.0, 1.2 Hz, 2H), 7.40 (dt, J = 8.1, 1.0 Hz, 2H), 7.28 (ddd, J = 7.9, 7.0, 1.2 Hz, 2H). ¹³C NMR (500 MHz, DMSO-d₆) δ 163.15, 148.50, 144.18, 143.87, 143.81, 139.73, 126.19, 120.81, 120.46, 120.08, 108.89. HRMS (ESI) m/z calcd. for C₁₇H₁₃N₄O [M + H]⁺ 289.1084, found | II |
| L-51, yield 94% | 289.1090. | |
| 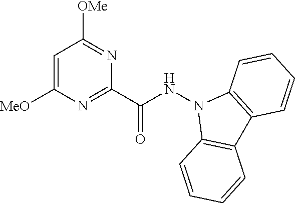 [SMILES: COc1cc(OC)nc(C(=O)Nn2c3ccccc3c3ccccc32)n1] | ¹H NMR (500 MHz, DMSO-d₆) δ 12.04 (s, 1H), 8.22 (dt, J = 7.8, 1.0 Hz, 2H), 7.47 (ddd, J = 8.2, 7.1, 1.1 Hz, 2H), 7.40 (dt, J = 8.1, 0.9 Hz, 2H), 7.29 (ddd, J = 7.9, 7.1, 1.0 Hz, 2H), 6.56 (s, 1H), 4.05 (s, 6H). ¹³C NMR (500 MHz, DMSO-d₆) δ 171.58, 161.77, 155.97, 139.69, 126.23, 120.81, 120.52, 120.15, 108.89, 91.95, 54.90. HRMS (ESI) m/z calcd. for C₁₉H₁₇N₄O₃ [M + H]⁺ 349.1295, found 349.1299. | II |
| L-52, yield 73% | | |
| 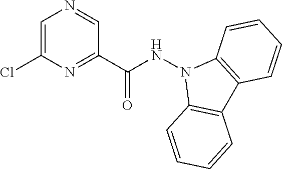 [SMILES: O=C(Nn1c2ccccc2c2ccccc21)c1cncc(Cl)n1] | ¹H NMR (500 MHz, DMSO-d₆) δ 12.32 (s, 1H), 9.28 (s, 1H), 9.18 (s, 1H), 8.21 (d, J = 7.7 Hz, 2H), 7.46 (dd, J = 6.3, 1.3 Hz, 4H), 7.29 (ddd, J = 8.0, 6.1, 2.0 Hz, 2H). ¹³C NMR (500 MHz, DMSO-d₆) δ 162.02, 148.33, 147.27, 143.54, 142.47, 139.69, 126.17, 120.85, 120.45, 120.14, 108.97. HRMS [ESI] m/z calcd. for [C₁₇H₁₁ClN₄O + Na]⁺ 345.0514, found 345.0519. | IV |
| L-53, yield 88% | | |
| 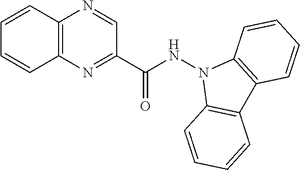 [SMILES: O=C(Nn1c2ccccc2c2ccccc21)c1cnc2ccccc2n1] | ¹H NMR (500 MHz, DMSO-d₆) δ 12.47 (s, 1H), 9.56 (s, 1H), 8.38-8.33 (m, 1H), 8.31- 8.27 (m, 1H), 8.23 (d, J = 7.8 Hz, 2H), 8.12-8.03 (m, 2H), 7.51-7.43 (m, 4H), 7.29 (ddd, J = 8.0, 6.5, 1.6 Hz, 2H). ¹³C NMR (500 MHz, DMSO-d₆) δ 163.36, 144.03, 143.43, 143.34, 139.94, 139.80, 132.53, 131.63, 129.73, 129.31, 126.19, 120.85, 120.47, 120.11, 109.05. HRMS (ESI) m/z calcd. for C₂₁H₁₅N₄O [M + H]⁺ 339.1240, found 339.1246. | IV |
| L-54, yield 91% | | |
| 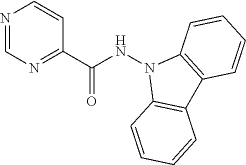 [SMILES: O=C(Nn1c2ccccc2c2ccccc21)c1ccncn1] | ¹H NMR (500 MHz, DMSO-d₆) δ 12.44 (s, 1H), 9.53 (d, J = 1.4 Hz, 1H), 9.20 (d, J = 5.1 Hz, 1H), 8.21 (dt, J = 7.8, 1.0 Hz, 2H), 8.15 (dd, J = 5.0, 1.4 Hz, 1H), 7.45 (ddd, J = 8.2, 7.0, 1.2 Hz, 2H), 7.39 (dt, J = 8.2, 0.9 Hz, 2H), 7.28 (ddd, J = 8.0, 7.1, 1.1 Hz, 2H). ¹³C NMR (500 MHz, DMSO-d₆) δ 163.00, 160.03, 158.27, 155.36, 139.62, 126.22, 120.84, 120.48, 120.13, 119.37, 108.89. HRMS (ESI) m/z calcd. for | IV |
| L-55, yield 83% | C₁₇H₁₃N₄O [M + H]⁺ 289.1084, found | |
| | 289.1089. | |
| 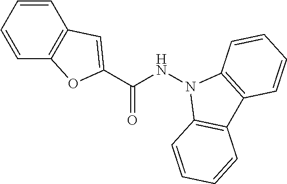 [SMILES: O=C(Nn1c2ccccc2c2ccccc21)c1cc2ccccc2o1] | ¹H NMR (500 MHz, DMSO-d₆) δ 12.18 (d, J = 2.7 Hz, 1H), 8.22 (dd, J = 8.0, 2.4 Hz, 2H), 7.93-7.87 (m, 2H), 7.79 (d, J = 8.3 Hz, 1H), 7.58 (t, J = 7.8 Hz, 1H), 7.50- 7.40 (m, 5H), 7.29 (t, J = 7.0 Hz, 2H). ¹³C NMR (500 MHz, DMSO-d₆) δ 158.49, 155.11, 147.20, 140.23, 128.15, 127.31, 126.72, 124.56, 123.65, 121.21, 120.93, 120.63, 112.54, 112.33, 109.24. HRMS (ESI) m/z calcd. For C₂₁H₁₅N₂O₂ (M + 1)⁺ requires 327.1135; found: 327.1128. | II |
| L-56, yield 55% | | |
| 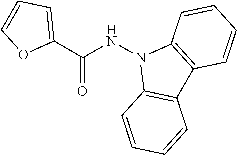 [SMILES: O=C(Nn1c2ccccc2c2ccccc21)c1ccco1] | ¹H NMR (500 MHz, DMSO-d₆) δ 11.80 (s, 1H), 8.20 (d, J = 7.7 Hz, 2H), 8.07 (s, 1H), 7.45 (t, J = 7.4 Hz, 3H), 7.37 (d, J = 8.1 Hz, 2H), 7.27 (t, J = 7.4 Hz, 2H), 6.82-6.78 (m, 1H). ¹³C NMR (500 MHz, DMSO-d₆) δ 157.82, 147.03, 145.95, 140.32, 126.69, 121.15, 120.91, 120.54, 116.46, 112.76, 109.15. HRMS (ESI) m/z calcd. for C₁₇H₁₅N₂O₂ [M + H]⁺ 279.1128 found 279.1133. | IV |
| L-57, yield 76% | | |
| 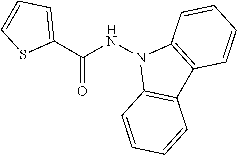 [SMILES: O=C(Nn1c2ccccc2c2ccccc21)c1cccs1] | ¹H NMR (500 MHz, DMSO-d₆) δ 11.89 (s, 1H), 8.21 (d, J = 7.8 Hz, 2H), 8.14 (q, J = 1.5 Hz, 1H), 8.02-7.98 (m, 1H), 7.47 (td, J = 7.6, 7.0, 1.2 Hz, 2H), 7.40 (d, J = 8.1 Hz, 2H), 7.34 (dd, J = 5.0, 3.7 Hz, 1H), 7.31- 7.25 (m, 2H). ¹³C NMR (500 MHz, DMSO- d₆) δ 161.45, 140.38, 136.50, 133.23, 130.60, 128.93, 126.74, 121.16, 120.95, 120.61, 109.16. HRMS (ESI) m/z calcd. for C₁₇H₁₅N₂OS [M + H]⁺ 295.0900 found | IV |
| L-58, yield 82% | 295.0902. | |
| 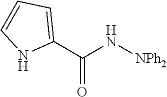 | ¹H NMR (400 MHz, DMSO-d₆) δ 11.63 (s, 1H), 10.74 (s, 1H), 7.29 (dd, J = 8.6, 7.2 Hz, 4H), 7.18-7.10 (m, 4H), 7.02-6.91 (m, 4H), 6.15 (q, J = 2.6 Hz, 1H). | VI |
| L-59, yield | | |
| 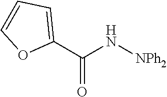 | ¹H NMR (500 MHz, DMSO-d₆) δ 11.18 (s, 1H), 7.92 (s, 1H), 7.28 (t, J = 7.6 Hz, 5H), 7.12 (d, J = 7.9 Hz, 4H), 6.97 (t, J = 7.2 Hz, 2H), 6.71-6.63 (m, 1H). ¹³C NMR (126 MHz, dmso) δ 157.87, 146.51, 146.46, 146.16, 129.54, 122.69, 119.21, 115.37, 112.44. | VII |
| L-60, yield | | |
| 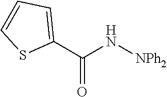 | ¹H NMR (500 MHz, DMSO-d₆) δ 11.24 (s, 1H), 7.93 (d, J = 3.7 Hz, 1H), 7.87 (d, J = 5.0 Hz, 1H), 7.29 (t, J = 7.7 Hz, 4H), 7.21 (t, J = 4.4 Hz, 1H), 7.13 (d, J = 8.0 Hz, 4H), 6.98 (t, J = 7.3 Hz, 2H). ¹³C NMR (126 MHz, dmso) δ 161.27, 146.13, 137.53, | VII |
| L61, yield | 132.52, 129.58, 128.74, 122.71, 120.20, 119.12. | |
| 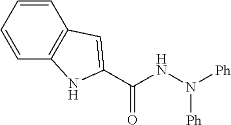 [SMILES: O=C(NN(c1ccccc1)c1ccccc1)c1cc2ccccc2[nH]1] | ¹H NMR (500 MHz, DMSO-d₆) δ 11.70 (s, 1H), 11.19 (s, 1H), 7.64 (d, J = 8.0 Hz, 1H), 7.43 (d, J = 8.3 Hz, 1H), 7.35-7.26 (m, 5H), 7.23-7.13 (m, 5H), 7.05 (t, J = 7.5 Hz, 1H), 6.98 (t, J = 7.3 Hz, 2H). | IV |
| L-62, yield 70% | | |
| 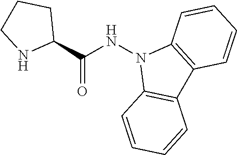 [SMILES: O=C(Nn1c2ccccc2c2ccccc21)[C@@H]1CCCN1] | ¹H NMR (500 MHz, DMSO-d₆) δ 11.12 (s, 1H), δ 8.17 (d, J = 7.7 Hz, 4H), 7.45 (t, J = 7.6 Hz, 4H), 7.29 (d, J = 8.1 Hz, 4H), 7.24 (t, J = 7.4 Hz, 4H), 3.93 (dd, J = 8.6, 5.6 Hz, 2H), 2.98 (dtd, J = 13.8, 10.1, 6.7 Hz, 4H), 2.18-2.08 (m, 2H), 1.99-1.91 (m, 2H), 1.87-1.78 (m, 2H), 1.73-1.67 (m, 1H). ¹³C NMR (500 MHz, DMSO) δ 175.24, 140.23, 126.41, 120.99, 120.77, 120.12, 109.04, 60.16, 47.36, 31.23, 26.27. HRMS | V |
| L-63, yield | (ESI) m/z calcd.For C₁₇H₁₈N₃O (M + 1)⁺ | |
| | requires 280.1446; found: 280.1444. | |
| 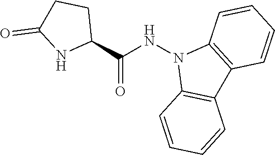 [SMILES: O=C1CC[C@@H](C(=O)Nn2c3ccccc3c3ccccc32)N1] | ¹H NMR (500 MHz, DMSO-d₆) δ 11.43 (s, 1H), 8.16 (d, J = 7.8 Hz, 2H), 8.12 (s, 1H), 7.45 (t, J = 7.6 Hz, 2H), 7.37 (d, J = 8.2 Hz, 2H), 7.24 (t, J = 7.4 Hz, 2H), 4.37 (dd, J = 8.9, 4.2 Hz, 1H), 2.56-2.49 (m, 1H), 2.31- 2.19 (m, 3H). ¹³C NMR (500 MHz, DMSO) δ 177.90, 173.07, 140.06, 126.60, 121.07, 120.90, 120.51, 109.17, 55.17, 29.76, 25.86. HRMS (ESI) m/z calcd.For C₁₇H₁₆N₃O₂ (M + 1)⁺ requires 294.1247; found: 294.1237. | V |
| L-64, yield | | |
| 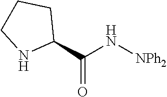 | ¹H NMR (500 MHz, DMSO-d₆) δ 11.20 (s, 1H), 7.30 (t, J = 7.8 Hz, 4H), 7.08 (d, J = 8.1 Hz, 4H), 7.01 (t, J = 7.3 Hz, 2H), 4.34 (t, J = 7.0 Hz, 1H), 3.22 (t, J = 6.9 Hz, 2H), 2.43- 2.30 (m, 1H), 1.99-1.86 (m, 3H). ¹³C NMR (126 MHz, dmso) δ 168.33, 145.79, 129.63, | V |
| L-65, yield | 123.09, 119.26, 58.49, 45.94, 29.38, 23.96. | |
| | HRMS (ESI) m/z calcd. for C₁₇H₂₀N₃O | |
| | [M + H]⁺ 282.1601 found 282.1601. | |
| 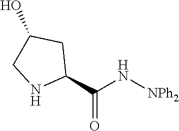 | ¹H NMR (500 MHz, DMSO-d₆) δ 10.55 (s, 1H), 7.25 (t, J = 7.9 Hz, 4H), 7.05 (d, J = 8.0 Hz, 4H), 6.94 (t, J = 7.3 Hz, 4H), 4.75 (s, 1H), 4.22 (s, 1H), 3.85 (t, J = 8.1 Hz, 1H), 2.93 (dd, J = 11.5, 4.3 Hz, 1H), 2.74 (d, J = 11.4 Hz, 1H), 1.95 (ddt, J = 11.4, 7.9, 1.8 Hz, 1H), 1.76 (ddd, J = 13.2, 8.4, 5.2 Hz, 1H). HRMS (ESI) m/z calcd. for | V |
| | C₁₇H₂₀N₃O₂ [M + H]⁺ 298.1550 found | |
| L-66, yield | 298.1557. | |

### Example 7

### Synthesis of 4,4′-Dimethoxydiphenylamine by Coupling Reaction of p-Chloroanisole and p-Methoxyaniline

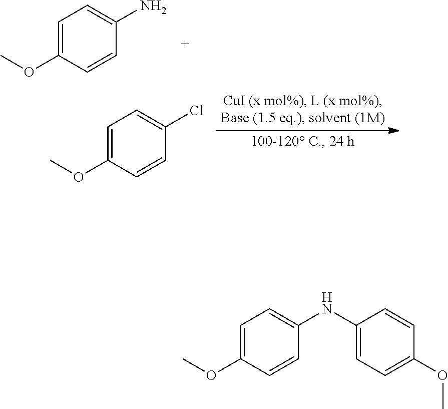

To a 10 mL sealed tube was added cuprous iodide (0.03-0.05 mmol), ligand (0.03-0.05 mmol), and base (1.5 mmol). The mixture was evacuated and replaced with argon atmosphere three times. p-Chloroanisole (1.0 mmol), p-methoxyaniline (1.5 mmol), and 1.0 mL of solvent were then added. The reaction system was stirred at 100 to 120° C. for 24 h. After cooling, the reaction system was poured into 10 mL of NH₃—NH₄Cl buffer solution with a pH of approximately 9. The mixture was extracted 3 times with 10 mL of ethyl acetate each time. The organic phases were combined, dried over anhydrous sodium sulfate, concentrated and purified by rapid column chromatography (Petroleum ether:ethyl acetate=50:1) to obtain the product 4,4′-dimethoxydiphenylamine.

¹H NMR (500 MHz, DMSO-d₆) δ 7.50 (s, 1H), 6.95-6.88 (m, 4H), 6.84-6.77 (m, 4H), 3.68 (s, 6H). ¹³C NMR (126 MHz, DMSO-d₆) δ 154.25, 137.90, 119.52, 114.77, 55.66.
the experimental results with different catalysts and ligand dosages, bases, solvents, and reaction temperatures are as follows:

| | Copper | | | Temper- | |
|---|---|---|---|---|---|
| Ligand | salt/ligand | | | ature | Yield |
| number | dosage | Base | Solvent | (° C.) | (%) |
| L10 | 5 mol %/ | K₃PO₄ | DMSO | 120 | 80 |
| | 5 mol % | | | | |
| L10 | 5 mol %/ | K₃PO₄ | DMAc | 120 | 79 |
| | 5 mol % | | | | |
| L10 | 5 mol %/ | K₃PO₄ | Sulfoane | 120 | 88 |
| | 5 mol % | | | | |
| L10 | 5 mol %/ | K₃PO₄ | NMP | 120 | 85 |
| | 5 mol % | | | | |
| L10 | 5 mol %/ | K₃PO₄ | DMI | 120 | 85 |
| | 5 mol % | | | | |
| L10 | 5 mol %/ | K₃PO₄ | Dioxane | 120 | 0 |
| | 5 mol % | | | | |
| L10 | 5 mol %/ | K₃PO₄ | t-BuOH | 120 | 10 |
| | 5 mol % | | | | |
| L10 | 5 mol %/ | K₃PO₄ | 2- | 120 | 72 |
| | 5 mol % | | Methoxyethanol | | |
| L10 | 5 mol %/ | K₃PO₄ | DMSO | 100 | 59 |
| | 5 mol % | | | | |
| L10 | 5 mol %/ | K₃PO₄ | Sulfolane | 100 | 53 |
| | 5 mol % | | | | |
| L10 | 5 mol %/ | K₃PO₄ | Glycol | 100 | 55 |
| | 5 mol % | | | | |
| L10 | 5 mol %/ | K₃PO₄ | Diglycol | 100 | 84 |
| | 5 mol % | | | | |
| L10 | 5 mol %/ | K₃PO₄ | PEG-200 | 100 | 88 |
| | 5 mol % | | | | |
| L10 | 5 mol %/ | K₃PO₄ | EtOH | 100 | 39 |
| | 5 mol % | | | | |
| L10 | 3 mol %/ | K₃PO₄ | Diglycol | 100 | 83 |
| | 3 mol % | | | | |
| L10 | 3 mol %/ | K₂CO₃ | Diglycol | 100 | 87 |
| | 3 mol % | | | | |
| L10 | 3 mol %/ | Cs₂CO₃ | Diglycol | 100 | 60 |
| | 3 mol % | | | | |
| L10 | 3 mol %/ | Na₂CO₃ | Diglycol | 100 | 60 |
| | 3 mol % | | | | |
| L10 | 3 mol %/ | Li₂CO₃ | Diglycol | 100 | 15 |
| | 3 mol % | | | | |
| L10 | 3 mol %/ | t-BuONa | Diglycol | 100 | 46 |
| | 3 mol % | | | | |
| L10 | 3 mol %/ | Na₃PO₄ | Diglycol | 100 | 61 |
| | 3 mol % | | | | |
| L10 | 3 mol %/ | Et₃N | Diglycol | 100 | 7 |
| | 3 mol % | | | | |
| L10 | 3 mol %/ | t-BuOK | Diglycol | 100 | 79 |
| | 3 mol % | | | | |
| L1 | 3 mol %/ | K₃PO₄ | Diglycol | 100 | 68 |
| | 3 mol % | | | | |
| L16 | 3 mol %/ | K₃PO₄ | Diglycol | 100 | 73 |
| | 3 mol % | | | | |
| L18 | 3 mol %/ | K₃PO₄ | Diglycol | 100 | 52 |
| | 3 mol % | | | | |
| L24 | 3 mol %/ | K₃PO₄ | Diglycol | 100 | 83 |
| | 3 mol % | | | | |
| L26 | 3 mol %/ | K₃PO₄ | Diglycol | 100 | 73 |
| | 3 mol % | | | | |
| L29 | 3 mol %/ | K₃PO₄ | Diglycol | 100 | 80 |
| | 3 mol % | | | | |
| L44 | 3 mol %/ | K₃PO₄ | Diglycol | 100 | 10 |
| | 3 mol % | | | | |
| L13 | 3 mol %/ | K₂CO₃ | PEG-200 | 100 | 96 |
| | 3 mol % | | | | |
| L10 | 3 mol %/ | K₂CO₃ | PEG-200 | 100 | 86 |
| | 3 mol % | | | | |
| L29 | 3 mol %/ | K₂CO₃ | PEG-200 | 100 | 82 |
| | 3 mol % | | | | |
| L18 | 3 mol %/ | K₂CO₃ | PEG-200 | 100 | 52 |
| | 3 mol % | | | | |
| L26 | 3 mol %/ | K₂CO₃ | PEG-200 | 100 | 73 |
| | 3 mol % | | | | |
| L27 | 3 mol %/ | K₂CO₃ | PEG-200 | 100 | 88 |
| | 3 mol % | | | | |
| L16 | 3 mol %/ | K₂CO₃ | PEG-200 | 100 | 92 |
| | 3 mol % | | | | |
| L24 | 3 mol %/ | K₂CO₃ | PEG-200 | 100 | 94 |
| | 3 mol % | | | | |
| L1 | 3 mol %/ | K₂CO₃ | PEG-200 | 100 | 64 |
| | 3 mol % | | | | |

### Example 8

### Synthesis of Corresponding Derivatives by Coupling Reaction of p-Chloroanisole and Various Aromatic Amines

To a 10 mL sealed tube was added cuprous iodide (0.03 mmol), ligand (the same as in Example 7, 0.03 mmol), and base (1.5 mmol). The mixture was evacuated and replaced with argon atmosphere three times. p-Chloroanisole (1.0 mmol), the corresponding aromatic amine (1.5 mmol), and 1.0 mL of solvent were then added. The reaction system was stirred at 100° C. for 24 h. After cooling, the reaction system was poured into 10 mL of NH₃—NH₄Cl buffer solution with a pH of approximately 9. The mixture was extracted 3 times with 10 mL of ethyl acetate each time. The organic phases were combined, dried over anhydrous sodium sulfate, concentrated and purified by rapid column chromatography to obtain the target compound.

This example uses different aromatic amines, and the results are listed in the following table:

| Product and yield | Characterization data of the product |
|---|---|
| 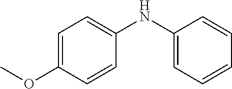 | ¹H NMR (500 MHz, DMSO-d₆) δ 7.84 (s, 1H), 7.18-7.12 (m, 2H), 7.07-7.00 (m, 2H), 6.93-6.83 (m, 4H), 6.70 (tt, J = 7.4, 1.2 Hz, 1H), 3.71 (s, 3H). ¹³C NMR (126 MHz, DMSO-d₆) δ 154.20, 145.56, 136.51, 129.54, 120.80, 118.66, 115.17, 114.94, 55.63. |
| Yield 89% | |
| 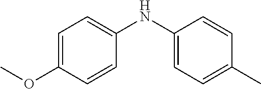 | ¹H NMR (500 MHz, DMSO-d₆) δ 7.69 (s, 1H), 6.98 (dd, J = 8.8, 2.5 Hz, 4H), 6.87-6.80 (m, 4H), 3.69 (s, 3H), 2.19 (s, 3H). ¹³C NMR (126 MHz, DMSO-d₆) δ 153.74, 142.85, 137.28, 129.96, 127.54, 119.80, 119.73, 115.91, 114.93, 55.63, 20.66. |
| Yield 90% | |
| 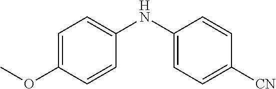 [SMILES: COc1ccc(Nc2ccc(C#N)cc2)cc1] | ¹H NMR (500 MHz, DMSO-d₆) δ 8.66 (s, 1H), 7.55-7.49 (m, 2H), 7.16-7.09 (m, 2H), 6.97-6.91 (m, 2H), 6.91- 6.85 (m, 2H). ¹³C NMR (126 MHz, DMSO-d₆) δ 156.02, 150.19, 134.10, 133.59, 123.80, 120.66, 115.13, 113.74, 98.22, 55.68. |
| Yield 83% | |
| 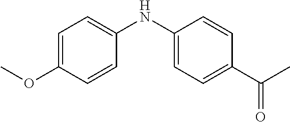 [SMILES: COc1ccc(Nc2ccc(C(C)=O)cc2)cc1] | ¹H NMR (500 MHz, DMSO-d₆) δ 8.58 (s, 1H), 7.80-7.74 (m, 2H), 7.16-7.09 (m, 2H), 6.97-6.90 (m, 2H), 6.90- 6.84 (m, 2H), 3.74 (s, 3H), 2.42 (s, 3H). ¹³C NMR (126 MHz, DMSO-d₆) δ 195.62, 155.67, 150.47, 134.18, 130.97, 127.09, 123.29, 115.07, 112.94, 55.67, 26.47. |
| Yield 70% | |
| 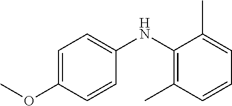 [SMILES: COc1ccc(Nc2c(C)cccc2C)cc1] | ¹H NMR (500 MHz, DMSO-d₆) δ 7.67 (s, 1H), 7.04-6.97 (m, 2H), 6.89-6.82 (m, 2H), 6.54-6.50 (m, 2H), 6.37- 6.33 (m, 1H), 3.70 (s, 3H), 2.15 (s, 6H). ¹³C NMR (126 MHz, DMSO-d₆) δ 154.10, 145.55, 138.36, 136.73, 120.95, 120.58, 114.90, 113.12, 55.62, 21.65. |
| Yield 80% | |
| 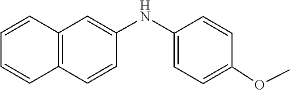 [SMILES: COc1ccc(Nc2ccc3ccccc3c2)cc1] | ¹H NMR (500 MHz, DMSO-d₆) δ 8.11 (s, 1H), 7.74-7.67 (m, 2H), 7.59 (dd, J = 8.5, 1.2 Hz, 1H), 7.33 (ddd, J = 8.2, 6.8, 1.3 Hz, 1H), 7.23 (d, J = 2.3 Hz, 1H), 7.22-7.13 (m, 4H), 6.96-6.89 (m, 2H). ¹³C NMR (126 MHz, DMSO-d₆) δ 154.64, 143.60, 136.17, 135.07, 129.22, 128.00, 127.84, 126.63, 126.29, 122.56, 121.48, 119.52, 115.06, 106.72, 55.68. |
| Yield 94% | |
| 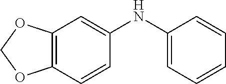 [SMILES: c1ccc(Nc2ccc3c(c2)OCO3)cc1] | ¹H NMR (500 MHz, DMSO-d₆) δ 7.88 (s, 1H), 7.21-7.13 (m, 2H), 6.97-6.90 (m, 2H), 6.80 (d, J = 8.3 Hz, 1H), 6.73 (tt, J = 7.3, 1.1 Hz, 1H), 6.68 (d, J = 2.2 Hz, 1H), 6.54 (dd, J = 8.3, 2.2 Hz, 1H), 5.95 (s, 2H). ¹³C NMR (126 MHz, DMSO-d₆) δ 148.15, 145.11, 141.71, 138.23, 129.57, 119.18, 115.88, 111.27, 108.95, 101.26, 101.14. |
| Yield 78% | |
| 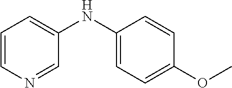 | ¹H NMR (500 MHz, DMSO-d₆) δ 8.19 (d, J = 2.8 Hz, 1H), 8.03 (s, 1H), 7.90 (dd, J = 4.6, 1.4 Hz, 1H), 7.24 (ddd, J = 8.3, 2.8, 1.4 Hz, 1H), 7.13 (dd, J = 8.3, 4.6 Hz, 1H), 7.08- 7.01 (m, 2H), 6.91-6.84 (m, 2H), 3.70 (s, 3H). ¹³C NMR (126 MHz, DMSO-d₆) δ 154.80, 142.09, 139.59, 138.03, 135.44, 124.19, 121.28, 120.44, 115.11, 55.67. |
| Yield 88% | |

### Example 9

### Synthesis of Phenol by Coupling Reaction of p-Chlorotoluene and H₂O

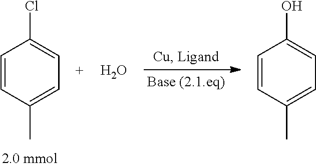

To a 10 mL sealed tube was added p-chlorotoluene (2.0 mmol), distilled water (1.0 mL), copper catalyst (0.04 mmol), ligand (0.04 mmol), and base (4.2 mmol). The mixture was evacuated and replaced with argon atmosphere three times. Then 1.0 mL of PEG-200 was added. The reaction was stirred evenly at 100° C. for 24 hours. After cooling, the pH of the system was adjusted to acidic with 3N HCl. The system was extracted with ethyl acetate, the organic phases were combined, concentrated, and then subjected to column chromatography to obtain the product 4-methylphenol: ¹H NMR (400 MHz, CDCl3) δ 7.04 (d, J=8.1 Hz, 2H), 6.74 (d, J=8.4 Hz, 2H), 4.94 (s, 1H), 2.28 (s, 3H); 13C NMR (100 MHz, CDCl3) δ 153.4, 130.2, 130.1, 115.2, 20.6.

The experimental results of different ligands are listed in the following table:

| Ligand | Yield |
|---|---|
| number | (%) |
| L-7 | 52% |
| L-8 | 58% |
| L-10 | 60% |
| L-11 | 18% |
| L-13 | 83% |
| L-16 | 94% |
| L-18 | 49% |
| L-21 | 30% |
| L-25 | 5% |
| L-32 | 2% |

The experimental results using different ligand dosages, bases, solvents, and reaction temperatures are as follows:

| | | | | Temper- | |
|---|---|---|---|---|---|
| | Copper | | | ature | Yield |
| Ligand | salt | Base | Solvent | (° C.) | (%) |
| L1 | Cu₂O | NaOH | DMSO/H₂O | 130 | 52 |
| (5 mol %) | (5 mol %) | | (1.0 mL/1.0 mL) | | |
| L1 | Cu₂O | NaOH | PEG-200/H₂O | 130 | 70 |
| (5 mol %) | (5 mol %) | | (1.0 mL/1.0 mL) | | |
| L1 | Cu₂O | NaOH | Glycol/H₂O | 130 | 29 |
| (5 mol %) | (5 mol %) | | (1.0 mL/1.0 mL) | | |
| L1 | Cu₂O | NaOH | DMF/H₂O | 130 | 43 |
| (5 mol %) | (5 mol %) | | (1.0 mL/1.0 mL) | | |
| L1 | Cu₂O | NaOH | 1-Butanol/H₂O | 130 | 5 |
| (5 mol %) | (5 mol %) | | (1.0 mL/1.0 mL) | | |
| L10 | Cu₂O | NaOH | PEG-200/H₂O | 130 | 90 |
| (5 mol %) | (5 mol %) | | (1.0 mL/1.0 mL) | | |
| L5 | Cu₂O | NaOH | PEG-200/H₂O | 130 | 83 |
| (5 mol %) | (5 mol %) | | (1.0 mL/1.0 mL) | | |
| L10 | CuI | NaOH | PEG-200/H₂O | 130 | 76 |
| (5 mol %) | (5 mol %) | | (1.0 mL/1.0 mL) | | |
| L10 | CuBr | NaOH | PEG-200/H₂O | 130 | 48 |
| (5 mol %) | (5 mol %) | | (1.0 mL/1.0 mL) | | |
| L10 | CuCl | NaOH | PEG-200/H₂O | 130 | 56 |
| (5 mol %) | (5 mol %) | | (1.0 mL/1.0 mL) | | |
| L10 | Cu(acac)₂ | NaOH | PEG-200/H₂O | 130 | 67 |
| (5 mol %) | (5 mol %) | | (1.0 mL/1.0 mL) | | |
| L10 | Cu(OAc)₂ | NaOH | PEG-200/H₂O | 130 | 82 |
| (5 mol %) | (5 mol %) | | (1.0 mL/1.0 mL) | | |
| L10 | Cu₂O | K₂CO₃ | PEG-200/H₂O | 130 | 58 |
| (5 mol %) | (5 mol %) | | (1.0 mL/1.0 mL) | | |
| L10 | Cu₂O | K₃PO₄ | PEG-200/H₂O | 130 | 75 |
| (5 mol %) | (5 mol %) | | (1.0 mL/1.0 mL) | | |
| L10 | Cu₂O | KOH | PEG-200/H₂O | 130 | 95 |
| (5 mol %) | (5 mol %) | | (1.0 mL/1.0 mL) | | |
| L10 | Cu₂O | t-BuONa | PEG-200/H₂O | 130 | 95 |
| (5 mol %) | (5 mol %) | | (1.0 mL/1.0 mL) | | |
| L10 | Cu₂O | t-BuOK | PEG-200/H₂O | 130 | 96 |
| (5 mol %) | (5 mol %) | | (1.0 mL/1.0 mL) | | |
| L24 | Cu₂O | KOH | PEG-200/H₂O | 100 | 92 |
| (2 mol %) | (2 mol %) | | (1.0 mL/1.0 mL) | | |
| L24 | Cu₂O | KOH | PEG-200/H₂O | 100 | 95 |
| (3 mol %) | (3 mol %) | | (1.0 mL/1.0 mL) | | |

### Example 10

### Synthesis of Corresponding Phenol Derivatives by Coupling Reaction of Aryl Chlorides with H₂O

To a 10 mL sealed tube was added aryl halide substrate (2.0 mmol), distilled water (1.0 mL), cuprous oxide (0.04 mmol), ligand (the same as in Example 9, 0.04 mmol), and base (4.2 mmol). The mixture was evacuated and replaced with argon atmosphere three times. Then 1.0 mL of PEG-200 was added. The reaction was stirred evenly at 100° C. for 24 hours. After cooling, the pH of the system was adjusted to acidic with 3N HCL. The system was extracted with ethyl acetate, and the organic phases were combined, concentrated, and then subjected to column chromatography to obtain the desired product.

This example uses different aryl chlorides, and the results are listed in the following table:

| Product and | |
|---|---|
| yield | Characterization data of the product |
| 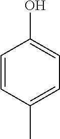 | ¹H NMR (400 MHz, CDCl₃) δ 7.04 (d, J = 8.1 Hz, 2H), 6.74 (d, J = 8.4 Hz, 2H), 4.94 (s, 1H), 2.28 (s, 3H); ¹³C NMR (100 MHz, CDCl₃) δ 153.4, 130.2, 130.1, 115.2, 20.6. |
| 92% | |
| 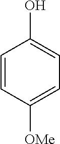 | ¹H NMR (400 MHz, CDCl₃) δ 6.87-6.69 (m, 4H), 5.05 (s, 1H), 3.77 (s, 3H); ¹³C NMR (100 MHz, CDCl₃) δ 153.8, 149.7, 116.2, 115.0, 56.0. |
| 84% | |
| 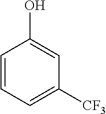 [SMILES: Oc1cccc(C(F)(F)F)c1] | ¹H NMR (400 MHz, CDCl₃) δ 7.35 (t, J = 8.0 Hz, 1H), 7.20 (d, J = 7.7 Hz, 1H), 7.12-7.07 (m, 1H), 7.01 (dd, J = 8.2, 2.2 Hz, 1H), 4.93 (br s, 1H); ¹³C NMR (100 MHz, CDCl₃) δ 155.8, 132.2 (q, J = 32.4 Hz), 130.4, 124.0 (q, J = 272.3 Hz), 119.0 (d, J = 1.0 Hz), 117.8 (q, J = 3.9 Hz), 112.5 (q, J = 3.8 Hz). |
| 56% | |
| 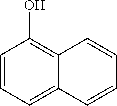 [SMILES: Oc1cccc2ccccc12] | ¹H NMR (400 MHz, CDCl₃) δ 8.20- 8.14 (m, 1H), 7.84-7.77 (m, 1H), 7.52-7.45 (m, 2H), 7.44 (d, J = 8.3 Hz, 1H), 7.33-7.26 (m, 1H), 6.80 (dd, J = 7.4, 0.5 Hz, 1H), 5.32 (s, 1H); ¹³C NMR (100 MHz, CDCl₃) δ 151.5, 134.8, 127.7, 126.5, 125.9, 125.3, 124.4, 121.6, 120.7, 108.7. |
| 82% | |
| 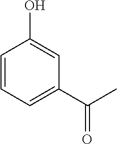 | ¹H NMR (400 MHz, CDCl₃) δ 7.56- 7.48 (m, 2H), 7.34 (t, J = 7.8 Hz, 1H), 7.10 (ddd, J = 8.1, 2.5, 0.9 Hz, 1H), 6.43 (s, 1H), 2.60 (s, 3H); ¹³C NMR (100 MHz, CDCl₃) δ 199.7, 156.6, 138.4, 130.0, 121.2, 121.1, 114.9, 26.9. |
| 88% | |
| 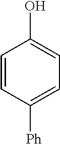 [SMILES: Oc1ccc(-c2ccccc2)cc1] | ¹H NMR (400 MHz, CDCl₃) δ 9.55 (s, 1H), 7.56 (d, J = 7.4 Hz, 2H), 7.48 (d, J = 8.6 Hz, 2H), 7.40 (t, J = 7.7 Hz, 2H), 7.27 (t, J = 7.3 Hz, 1H), 6.87 (d, J = 8.6 Hz, 2H); ¹³C NMR (100 MHz, DMSO-d₆) δ 157.2, 140.2, 131.0, 128.8, 127.7, 126.3, 126.0, 115.7. |
| 83% | |
| 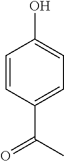 | ¹H NMR (400 MHz, CDCl₃) δ 8.67 (s, 1H), 7.92 (d, J = 8.7 Hz, 2H), 6.98 (d, J = 8.7 Hz, 2H), 2.59 (s, 3H); ¹³C NMR (100 MHz, CDCl₃) δ 199.4, 162.0, 131.5, 129.3, 115.8, 26.4. |
| 91% | |
| 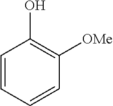 | ¹H NMR (500 MHz, CDCl₃) δ 6.93- 6.92 (m, 1H), 6.88-6.84 (m, 3H), 5.63 (s, 1H), 3.87 (s, 3H); ¹³C NMR (126 MHz, CDCl₃) δ 146.76, 145.83, 121.60, 120.29, 114.73, 110.94, 56.00. |
| 42% | |

### Example 11

### Synthesis of Phenols by Coupling Reaction of p-Bromotoluene and H₂O Under Room Temperature to Near Room Temperature Conditions

To a 10 mL sealed tube was added p-bromotoluene (4.0 mmol), cuprous oxide (1.0 mol %), ligand (1.0 mol %), and potassium tert-butoxide (8.4 mmol). The mixture was evacuated and replaced with argon atmosphere three times. Then 2.0 mL of solvent and 2.0 mL of distilled water were added. The reaction was stirred evenly at 30 to 40° C. for 24 hours. After cooling, the pH of the system was adjusted to acidic with 3N HCl. The system was extracted with ethyl acetate, and the organic phases were combined, concentrated, and then subjected to column chromatography to obtain the product 4-methylphenol: ¹H NMR (400 MHz, CDCl3) δ 7.04 (d, J=8.1 Hz, 2H), 6.74 (d, J=8.4 Hz, 2H), 4.94 (s, 1H), 2.28 (s, 3H); ¹³C NMR (100 MHz, CDCl₃) δ 153.4, 130.2, 130.1, 115.2, 20.6.

The experimental results of different ligands are listed in the following table:

| Ligand | Yield |
|---|---|
| number | (%) |
| L-8 | 42% |
| L-10 | 58% |
| L-11 | 24% |
| L-13 | 78% |
| L-15 | 60% |
| L-16 | 80% |
| L-24 | 95% |
| L-27 | 85% |
| L-31 | 3% |
| L-32 | 2% |

The experimental results using different solvents are as follows:

| | | | | Temper- | |
|---|---|---|---|---|---|
| | Copper | | | ature | Yield |
| Ligand | salt | Base | Solvent 1 | (° C.) | (%) |
| L27 | Cu₂O | NaOH | 2-methoxyethanol | 40 | 66 |
| (1 mol %) | (1 mol %) | | | | |
| L27 | Cu₂O | NaOH | EtOH | 40 | 68 |
| (1 mol %) | (1 mol %) | | | | |
| L27 | Cu₂O | NaOH | PEG-200 | 40 | 78 |
| (1 mol %) | (1 mol %) | | | | |
| L27 | Cu₂O | NaOH | THF | 40 | 43 |
| (1 mol %) | (1 mol %) | | | | |
| L27 | Cu₂O | NaOH | acetone | 40 | 50 |
| (1 mol %) | (1 mol %) | | | | |
| L27 | Cu₂O | NaOH | 1,4-dioxane | 40 | 47 |
| (1 mol %) | (1 mol %) | | | | |
| L27 | Cu₂O | NaOH | tBuOH | 40 | 55 |
| (1 mol %) | (1 mol %) | | | | |
| L27 | Cu₂O | NaOH | Methyl isobutyl | 40 | 66 |
| (1 mol %) | (1 mol %) | | ketone | | |
| L27 | Cu₂O | NaOH | MeOH | 40 | 51 |
| (1 mol %) | (1 mol %) | | | | |
| L27 | Cu₂O | NaOH | NMP | 40 | 34 |
| (1 mol %) | (1 mol %) | | | | |
| L27 | Cu₂O | NaOH | sulfolane | 40 | 56 |
| (1 mol %) | (1 mol %) | | | | |
| L27 | Cu₂O | NaOH | DMA | 40 | 32 |
| (1 mol %) | (1 mol %) | | | | |
| L27 | Cu₂O | NaOH | propylene | 40 | 57 |
| (1 mol %) | (1 mol %) | | carbonate | | |
| L27 | Cu₂O | NaOH | ethyl acetate | 40 | 33 |
| (1 mol %) | (1 mol %) | | | | |
| L24 | Cu₂O | NaOH | PEG-200 | 80 | 95 |
| (1 mol %) | (1 mol %) | | | | |
| L24 | Cu₂O | tBuOK | PEG-200 | 30 | 88 |
| (1 mol %) | (1 mol %) | | | | |

### Example 12

### Synthesis of Corresponding Phenol Derivatives by Coupling Reaction of Aryl Bromide and H₂O at Room Temperature

To a 10 mL sealed tube were added aryl bromide (4.0 mmol), cuprous oxide (1.0 mol %), ligand (the same as in Example 11, 1.0 mol %), and potassium tert-butoxide (8.4 mmol). The mixture was evacuated and replaced with argon atmosphere three times. 2.0 mL of PEG-200 and 2.0 mL of distilled water were added. The reaction was stirred evenly at 30° C. for 24 hours. After cooling, the pH of the system was adjusted to acidic with 3N HCl, extracted with ethyl acetate, and the organic phases were combined, concentrated, and purified by column chromatography to obtain the target compound.

| Product and yield | Characterization data of the product |
|---|---|
| 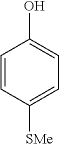 | ¹H NMR (400 MHz, CDCl₃) δ 7.25-7.20 (m, 2H), 6.81-6.76 (m, 2H), 5.09 (s, 1H), 2.44 (s, 3H); ¹³C NMR (100 MHz, CDCl₃) δ 154.2, 130.5, 129,0, 116.2, 18.2. |
| 80% | |
| 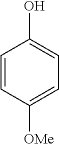 | ¹H NMR (400 MHz, CDCl₃) δ 6.87-6.69 (m, 4H), 5.05 (s, 1H), 3.77 (s, 3H); ¹³C NMR (100 MHz, CDCl₃) δ 153.8, 149.7, 116.2, 115.0, 56.0. |
| 93% | |
| 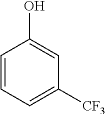 [SMILES: Oc1cccc(C(F)(F)F)c1] | ¹H NMR (400 MHz, CDCl₃) δ 7.35 (t, J = 8.0 Hz, 1H), 7.20 (d, J = 7.7 Hz, 1H), 7.12-7.07 (m, 1H), 7.01 (dd, J = 8.2, 2.2 Hz, 1H), 4.93 (br s, 1H); ¹³C NMR (100 MHz, CDCl₃) δ 155.8, 132.2 (q, J = 32.4 Hz), 130.4, 124.0 (q, J = 272.3 Hz), 119.0 (d, J = 1.0 Hz), 117.8 (q, J = 3.9 Hz), 112.5 (q, J = 3.8 Hz). |
| 58% | |
| 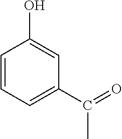 | ¹H NMR (400 MHz, CDCl₃) δ 7.56-7.48 (m, 2H), 7.34 (t, J = 7.8 Hz, 1H), 7.10 (ddd, J = 8.1, 2.5, 0.9 Hz, 1H), 6.43 (s, 1H), 2.60 (s, 3H); ¹³C NMR (100 MHz, CDCl₃) δ 199.7, 156.6, 138.4, 130.0, 121.2, 121.1, 114.9, 26.9. |
| 78% | |
| 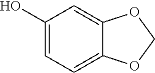 | ¹H NMR (400 MHz, CDCl₃) δ 6.65 (d, J = 8.3 Hz, 1H), 6.43 (d, J = 2.5 Hz, 1H), 6.25 (dd, J= 8.3, 2.5 Hz, 1H), 5.91 (s, 2H), 4.69 (s, 1H); ¹³C NMR (100 MHz, CDCl₃) δ 150.6, 148.2, 141.5, 108.3, 106.9, 101.2, 98.4. |
| 84% | |
| 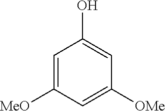 [SMILES: COc1cc(O)cc(OC)c1] | ¹H NMR (400 MHz, CDCl₃) δ 6.08(t, J = 2.1 Hz, 1H),6.04(d, J= 2.2 Hz,2H), 5.83 (s, 1H), 3.74 (s, 6H); ¹³C NMR (100 MHz, CDCl₃) δ 161.7,157.6, 94.5, 93.3, 55.5. |
| 84% | |
| 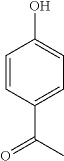 | ¹H NMR (400 MHz, CDCl₃) δ 8.67 (s, 1H), 7.92 (d, J = 8.7 Hz, 2H), 6.98 (d, J = 8.7 Hz, 2H), 2.59 (s, 3H); ¹³C NMR (100 MHz, CDCl₃) δ 199.4, 162.0, 131.5, 129.3, 115.8, 26.4. |
| 80% | |
| 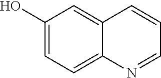 [SMILES: Oc1ccc2ncccc2c1] | ¹H NMR (400 MHz, DMSO-d₆) δ 10.03 (s, 1H), 8.65 (dd, J = 4.1, 1.5 Hz, 1H), 8.12 (d, J = 7.7 Hz, 1H), 7.87 (d, J = 9.1 Hz, 1H), 7.38 (dd, J = 8.3, 4.2 Hz, 1H), 7.32 (dd, J = 9.1, 2.7 Hz, 1H), 7.15 (d, J = 2.6 Hz, 1H); ¹³C NMR (100 MHz, |
| | DMSO-d₆) δ 155.5, 147.1, 143.0, 134.1, |
| 25% | 130.4, 129.3, 122.0, 121.4, 108.4. |

### Example 13

### Synthesis of Corresponding Phenol Derivatives by Coupling Reaction of Aryl Iodides with H₂O at Room Temperature

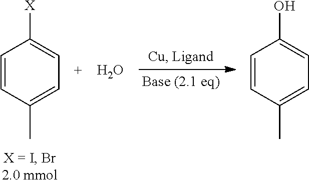

To a 10 mL sealed tube was added 4-methyliodobenzene (4.0 mmol), distilled water (2.0 mL), copper catalyst (0.05 to 1.0 mol %), ligand (0.05 to 1.0 mol %), and potassium tert-butoxide (8.4 mmol). The mixture was evacuated and replaced with argon atmosphere three times. Then 2.0 mL of PEG-200 was added. The reaction was stirred evenly at 30° C. for 24 hours. After cooling, the 10 pH of the system was adjusted to acidic with 3N HCl, extracted with ethyl acetate. The organic phases were combined, concentrated, and then subjected to column chromatography to obtain the product 4-methylphenol: ¹H NMR (400 MHz, CDCl₃) δ 7.04 (d, J=8.1 Hz, 2H), 6.74 (d, J=8.4 Hz, 2H), 4.94 (s, 1H), 2.28 (s, 3H); ¹³C NMR (100 MHz, CDCl₃) δ 153.4, 130.2, 130.1, 115.2, 20.6.

The experimental results of different ligands are listed in the following table:

| | | Copper | | | |
|---|---|---|---|---|---|
| Ligand | | salt/ligand | | | Yield |
| number | Aryl halides | dosage | Base | Solvent | (%) |
| L24 | 4- | 1 mol %/ | tBuOK | PEG-200/H₂O | 94 |
| | methyliodo- | 1 mol % | | (2.0 mL/2.0 mL) | |
| | benzene | | | | |
| L27 | 4- | 1 mol %/ | tBuOK | PEG-200/H₂O | 98 |
| | methyliodo- | 1 mol % | | (2.0 mL/2.0 mL) | |
| | benzene | | | | |
| L24 | 4- | 0.5 mol %/ | tBuOK | PEG-200/H₂O | 90 |
| | methyliodo- | 0.5 mol % | | (2.0 mL/2.0 mL) | |
| | benzene | | | | |

### Example 14

### Coupling of p-Chloroanisole with Sodium Phenylsulfinate

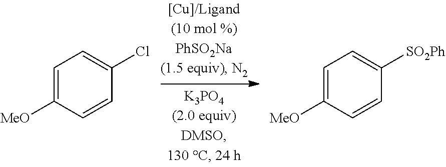

To a 10 mL sealed tube was added sodium phenylsulfinate (1.5 mmol), copper catalyst (0.1 mmol), ligand (0.1 mmol), and potassium phosphate (2.0 mmol). The mixture was vacuumed and replaced with argon atmosphere three times. Then p-chloroanisole (1.0 mmol) and 1 mL DMSO were added. The reaction was stirred evenly at 130° C. for 24 hours. After cooling, the contents of the sealed tube were washed out with ethyl acetate and filtered through a silica gel and diatomaceous earth column. The filtrate was concentrated and subjected to column chromatography to obtain the product diphenyl sulfone: ¹H NMR (500 MHz, Chloroform-d) δ 7.84 (d, J=8.9 Hz, 2H), 7.00 (d, J=8.9 Hz, 2H), 3.86 (s, 3H), 3.01 (s, 3H). ¹³C NMR (126 MHz, cdcl₃) δ 163.68, 132.25, 129.51, 114.49, 55.70, 44.83.

The results obtained by screening with different copper salts and ligands are listed in the table below.

| Ligand | Copper | |
|---|---|---|
| number | catalyst | Yield |
| L32 | CuI | 79% |
| L32 | CuCl | 60% |
| L32 | CuBr | 63% |
| L32 | Cu₂O | 15% |
| L32 | CuCN | 9% |
| L32 | CuTc | 83% |
| L32 | CuBr₂ | 13% |
| L32 | Cu(acac)₂ | 6% |
| L32 | Cu(OTf)₂ | 10% |
| L32 | Cu | 66% |
| L33 | CuTc | 68% |
| L34 | CuTc | 80% |
| L35 | CuTc | 81% |
| L37 | CuTc | 78% |

### Example 15

### Coupling of Aryl Chlorides with Sodium Arylsulfinate and Sodium Alkylsulfinate

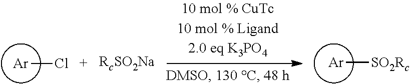

To a 10 mL sealed tube was add sodium alkyl- or aryl-sulfinate (1.5 mmol), CuTc (0.1 mmol), ligand (the same as in Example 14, 0.1 mmol), and potassium phosphate (2.0 mmol). The mixture was vacuumed and replaced with argon atmosphere three times. Then, aryl chloride (1.0 mmol) and 1 mL DMSO were added. The reaction was stirred evenly at 130° C. for 48 hours. After cooling, the contents of the sealed tube were washed out with ethyl acetate and filtered through a silica gel and diatomaceous earth column. The filtrate was concentrated and then subjected to column chromatography to obtain the corresponding product.

| Structure and yield of the | |
|---|---|
| product | Characterization data of the product |
| 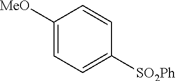 | ¹H NMR (500 MHz, Chloroform-d) δ 7.84 (d, J = 8.9 Hz, 2H), 7.00 (d, J = 8.9 Hz, 2H), 3.86 (s, 3H), 3.01 (s, 3H). ¹³C NMR (126 MHz, CDCl₃) δ 163.68, 132.25, 129.51, 114.49, 55.70, 44.83. |
| Chemical Formula: C₁₃H₁₂O₃S | |
| Molecular Weight: 248.2960 | |
| 93% | |
| 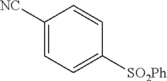 | ¹H NMR (500 MHz, Chloroform-d) δ 8.05 (d, J= 8.5 Hz, 2H), 7.94 (d, J = 7.1 Hz, 2H), 7.79 (d, J = 8.5 Hz, 2H), 7.62 (t, J = 7.4 Hz, 1H), 7.54 (t, J = 7.8 Hz, 2H). ¹³C NMR (126 MHz, CDCl₃) δ 145.85, 140.12, 134.03, 133.08, 129.65, 128.27, 127.98, 117.14, 116.92. |
| Chemical Formula: C₁₃H₉NO₂S | |
| Molecular Weight: 243.2800 | |
| 80% | |
| 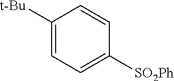 | ¹H NMR (500 MHz, Chloroform-d) δ 7.95 (d, J = 7.1 Hz, 2H), 7.86 (d, J = 8.6 Hz, 2H), 7.54 (t, J = 7.3 Hz, 1H), 7.49 (td, J = 8.5, 1.7 Hz, 4H), 1.30 (s, 9H). ¹³C NMR (126 MHz, CDCl₃) δ 157.06, 141.93, 138.52, 132.99, 129.20, 127.58, 127.51, 126.30, 35.16, 31.01. |
| Chemical Formula: C₁₆H₁₈O₂S | |
| Molecular Weight: 274.3780 | |
| 96% | |
| 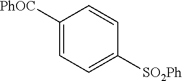 | ¹H NMR (500 MHz, Chloroform-d) δ 8.05 (d, J = 8.5 Hz, 2H), 7.98 (dt, J = 7.3, 1.3 Hz, 2H), 7.87 (d, J = 8.5 Hz, 2H), 7.76 (d, J = 7.0 Hz, 2H), 7.66-7.57 (m, 2H), 7.54 (t, J = 7.6 Hz, 2H), 7.49 (t, J = 7.8 Hz, 2H). ¹³C NMR (126 MHz, CDCl₃) δ 195.19, 144.69, 141.71, 140.85, 136.36, 133.64, 133.31, 130.48, |
| Chemical Formula: C₁₉H₁₄O₃S | 130.10, 129.48, 128.59, 127.90, 127.65. |
| Molecular Weight: 322.3780 | |
| 81% | |
| 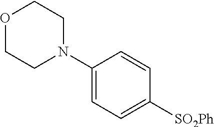 | ¹H NMR (500 MHz, Chloroform-d) δ 7.88 (dd, J = 7.9, 2.9 Hz, 2H), 7.78 (dd, J = 9.0, 3.1 Hz, 2H), 7.46 (ddt, J = 15.9, 7.8, 4.1 Hz, 3H), 6.87 (d, J = 8.4 Hz, 2H), 3.80 (d, J = 3.8 Hz, 4H), 3.25 (d, J = 3.7 Hz, 4H). ¹³C NMR (126 MHz, CDCl₃) δ 154.04, 142.79, 132.58, 129.97, 129.47, 129.11, 127.10, 113.82, 66.42, 47.34. |
| Chemical Formula: C₁₆H₁₇NO₃S | |
| Molecular Weight: 303.3760 | |
| 85% | |
| 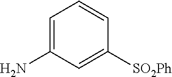 | ¹H NMR (500 MHz, Chloroform-d) δ 7.91 (dd, J = 7.3, 1.8 Hz, 2H), 7.53 (t, J = 7.2 Hz, 1H), 7.47 (t, J = 7.4 Hz, 2H), 7.29-7.13 (m, 3H), 6.79 (d, J = 7.4 Hz, 1H), 3.91 (s, 2H). ¹³C NMR (126 MHz, CDC13) 8 147.42, 142.14, 141.68, 133.06, 130.18, 129.19, 127.54, 119.37, 117.12, 113.12. |
| Chemical Formula: C₁₂H₁₁NO₂S | |
| Molecular Weight: 233.2850 | |
| 89% | |
| 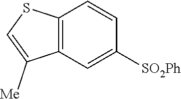 | ¹H NMR (500 MHz, Chloroform-d) δ 8.35 (t, J = 1.7 Hz, 1H), 7.98 (d, J = 8.1 Hz, 2H), 7.90 (d, J = 8.5 Hz, 1H), 7.82 (dt, J = 8.5, 1.6 Hz, 1H), 7.55-7.43 (m, 3H), 7.18 (s, 1H), 2.46 (s, 3H). ¹³C NMR (126 MHz, CDCl₃) δ 144.98, 142.03, 139.58, 137.30, 133.05, 132.89, 129.28, 127.52, 124.36, 123.75, 122.25, 121.69, 13.84. |
| Chemical Formula: C₁₅H₁₂O₂S₂ | |
| Molecular Weight: 288.3790 | |
| 93% | |
| 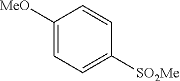 [SMILES: COc1ccc(S(C)(=O)=O)cc1] | ¹H NMR (500 MHz, Chloroform-d) δ 7.84 (d, J = 8.9 Hz, 2H), 7.00 (d, J = 8.9 Hz, 2H), 3.86 (s, 3H), 3.01 (s, 3H). ¹³C NMR (126 MHz, CDCl₃) δ 163.68, 132.25, 129.51, 114.49, 55.70, 44.83. |
| Chemical Formula: C₈H₁₀O₃S | |
| Molecular Weight: 186.2250 | |
| 90% | |
| 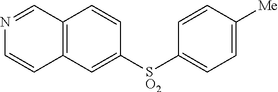 [SMILES: Cc1ccc(S(=O)(=O)c2ccc3cnccc3c2)cc1] | ¹H NMR (500 MHz, Chloroform-d) δ 9.30 (s, 1H), 8.64 (d, J = 5.7 Hz, 1H), 8.52 (d, J = 1.8 Hz, 1H), 8.05 (d, J = 8.6 Hz, 1H), 7.96 (dd, J = 8.7, 1.7 Hz, 1H), 7.88 (d, J = 8.3 Hz, 2H), 7.77 (d, J = 5.7 Hz, 1H), 7.30 (d, J = 8.1 Hz, 2H), 2.37 (s, 3H). ¹³C NMR (126 MHz, CDCl₃) δ 152.52, 144.83, 144.64, 143.43, 137.68, 134.90, 130.13, 129.45, 129.30, 128.01, |
| Chemical Formula: C₁₆H₁₃NO₂S | 127.14, 124.24, 121.33, 21.59. |
| Molecular Weight: 283.3450 | |
| 78% | |
| 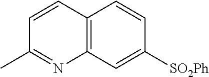 | ¹H NMR (500 MHz, Chloroform-d) δ 8.64 (s, 1H), 8.05 (d, J = 8.5 Hz, 1H), 8.00 (d, J = 7.2 Hz, 2H), 7.92 (dd, J = 8.5, 1.8 Hz, 1H), 7.86 (d, J = 8.6 Hz, 1H), 7.54 (t, J = 7.4 Hz, 1H), 7.48 (t, J = 7.5 Hz, 2H), 7.39 (d, J = 8.5 Hz, 1H), 2.74 (s, 3H). ¹³C NMR (126 MHz, CDCl₃) δ 161.25, 146.80, 142.17, 141.19, |
| Chemical Formula: C₁₆H₁₃NO₂S | 135.98, 133.37, 129.44, 129.34, 129.23, 128.68, |
| Molecular Weight: 283.3450 | 127.92, 124.66, 122.57, 25.39. |
| 77% | |
| 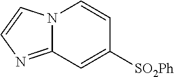 | ¹H NMR (500 MHz, Chloroform-d) δ 8.30 (s, 1H), 8.20 (d, J = 7.1 Hz, 1H), 7.95 (d, J = 8.1 Hz, 2H), 7.80 (s, 1H), 7.70 (s, 1H), 7.58 (t, J = 7.4 Hz, 1H), 7.51 (t, J = 7.6 Hz, 2H), 7.18 (dd, J = 7.2, 1.8 Hz, 1H). ¹³C NMR (126 MHz, CDCl₃) δ 143.21, 140.46, 137.06, 136.72, 133.72, 129.50, 127.80, 126.68, |
| Chemical Formula: C₁₃H₁₀N₂O₂S | 118.95, 114.39, 109.14. |
| Molecular Weight: 258.2950 | |
| 65% | |
| 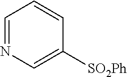 | ¹H NMR (500 MHz, Chloroform-d) δ 9.14 (s, 1H), 8.77 (d, J = 4.7 Hz, 1H), 8.21 (d, J = 8.1 Hz, 1H), 7.96 (d, J = 7.8 Hz, 2H), 7.60 (t, J = 7.4 Hz, 1H), 7.53 (t, J = 7.6 Hz, 2H), 7.44 (dd, J = 8.1, 4.8 Hz, 1H). ¹³C NMR (126 MHz, CDCl₃) δ 153.62, 148.66, 140.71, 138.31, 135.26, 133.84, 129.59, 127.79, 123.88. |
| Chemical Formula: C₁₁H₉NO₂S | |
| Molecular Weight: 219.2580 | |
| 63% | |
| 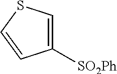 | ¹H NMR (500 MHz, Chloroform-d) δ 8.09 (d, J = 3.1 Hz, 1H), 7.96 (d, J = 7.4 Hz, 2H), 7.58 (t, J = 7.4 Hz, 1H), 7.51 (t, J = 7.6 Hz, 2H), 7.38 (dd, J = 5.2, 3.1 Hz, 1H), 7.33 (d, J = 5.1 Hz, 1H). ¹³C NMR (126 MHz, CDCl₃) δ 141.98, 141.59, 133.29, 131.58, 129.30, 128.40, 127.44, 125.84. |
| Chemical Formula: C₁₀H₈O₂S₂ | |
| Molecular Weight: 224.2920 | |
| 61% | |

All references mentioned in the present invention are cited as references in this application, as if each reference was cited separately. In addition, it should be understood that after reading the above teaching of the present invention, those skilled in the art can make various modifications or changes to the present invention, and these equivalent forms also fall within the scope of the appended claims of the present invention.

1. The method of a compound as shown in formula I, and isomers thereof: 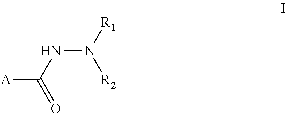
wherein, A is a saturated or unsaturated C5-C6 carbocyclyl, or a saturated or unsaturated 5 - or 6-membered carbon heterocarbocyclyl, or a benzo saturated or unsaturated 5- or 6-membered carbon heterocarbocyclyl, wherein the carbon heterocarbocyclyl has 1 to 5 heteroatoms selected from the group consisting of: N, O, and S; A is optionally substituted by substituents selected from the group consisting of: hydroxyl, nitro,—NRaRb, halogen, C1-4 alkyl, C1-4 alkoxy, phenyl, and oxo (i.e., two hydrogen atoms on the same carbon atom in the group are replaced by ═O);
Ra and Rb are each independently selected from the group consisting of: H, hydroxyl, amino, halogen, and C1-4 alkyl;
R₁ and R₂ are each independently selected from the group consisting of: H, substituted or unsubstituted C1-C6 alkyl, substituted or unsubstituted C1-C6 alkoxy, substituted or unsubstituted C3-C6 cycloalkyl, substituted or unsubstituted 3- to 6-membered heterocyclyl, substituted or unsubstituted C6-C20 aryl, substituted or unsubstituted 5- to 20-membered heteroaryl, and substituted or unsubstituted benzyl; or, R₁ and R₂, together with the N atom to which they are attached, form a 3- to 20-membered saturated or unsaturated carbocycle or heterocycle (such as substituted or unsubstituted pyrrole, indole, azoles, benzazole, and aromatic heterocycle);
characterized in that it is used as a ligand in copper-catalyzed coupling reactions of aryl halide; wherein, the aryl halide is selected from the group consisting of: aryl chloride, aryl bromide, aryl iodide, or a combination thereof;
unless otherwise specified, the “substituted” refers to one or more hydrogen atoms in the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl (alkyl-CO—), sulfonate, or two hydrogen atoms on two adjacent carbon atoms in the group are replaced by —(CH₂)ₙ— (n is 1, 2, 3, 4, 5 or 6).

2. The method according to claim 1, characterized in that A is selected from the group consisting of: substituted or unsubstituted indolyl, substituted or unsubstituted benzimidazolyl, substituted or unsubstituted imidazolyl, substituted or unsubstituted thiazolyl, substituted or unsubstituted benzothiazolyl, substituted or unsubstituted thienyl, substituted or unsubstituted benzothienyl, substituted or unsubstituted furyl, substituted or unsubstituted benzofuranyl, substituted or unsubstituted saturated or unsaturated 5- to 6-membered heterocyclyl containing 1 or 2 N atoms (such as pyridyl, pyrazinyl, pyrimidinyl, pyridazinyl, azolyl, tetrahydropyrrolyl, hexahydropiperidinyl), substituted or unsubstituted phenyl, substituted or unsubstituted naphthyl, substituted or unsubstituted benzyl, substituted or unsubstituted quinolinyl, and substituted or unsubstituted isoquinolinyl;
the “substituted” refers to one or more hydrogen atoms being substituted with substituents selected from the group consisting of: hydroxyl, nitro, —NRaRb, halogen, C1-4 alkyl, C1-4 alkoxy, phenyl, and oxo (i.e., two hydrogen atoms on the same carbon atom in the group are replaced by ═O), wherein, Ra and Rb are each independently selected from the group consisting of: H, hydroxyl, amino, halogen, and C1-4 alkyl.

3. The method according to claim 1, characterized in that the structure of the compound is as shown in formula II: 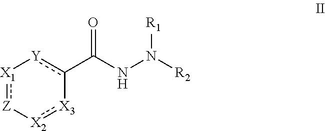
R₁ and R₂ are each independently selected from the group consisting of: H, substituted or unsubstituted C1-C6 alkyl, substituted or unsubstituted C6-C10 aryl, substituted or unsubstituted benzyl; R₁ and R₂, together with the N atom to which they attached form a 3-20 membered saturated or unsaturated carbocycle or heterocycle;
X₁ is selected from CR₃, N, and NH;
X₂ is selected from CR₄, and N;
X₃ is selected from CR₅, and N;
Y is selected from CR₆, NH, N, O, and S;
Z is selected from CₙR₇, N, and NH, wherein n is 0 or 1;
R₃, R₄, R₅, R₆, and R₇ are each independently H, halogen, hydroxyl, nitro, C₁₋₃ alkoxy (C₁₋₃ alkyl-O—), —NRaRb, oxo (i.e. two hydrogen atoms on the same carbon atom in the group are replaced by ═O); wherein, Ra and Rb are each independently selected from the group consisting of: H, hydroxyl, amino, halogen, and C1-4 alkyl;
or, any two of R₃, R₄, R₅, R₆, and R₇ that are located on adjacent ring atoms together with the carbon atom to which they are attached form a substituted or unsubstituted C6-C10 aryl, or a substituted or unsubstituted 5- to 8-membered heteroaryl;
the “substituted” refers to one or more hydrogen atoms in the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl group (alkyl-CO—), sulfonate, oxo (i.e. two hydrogen atoms on the same carbon atom in the group are replaced by ═O), or two hydrogen atoms on two adjacent carbon atoms in the group are replaced by —(CH₂)ₙ— (n is 1, 2, 3, 4, 5 or 6).

4. The method according to claim 1, characterized in that the compound is selected from:

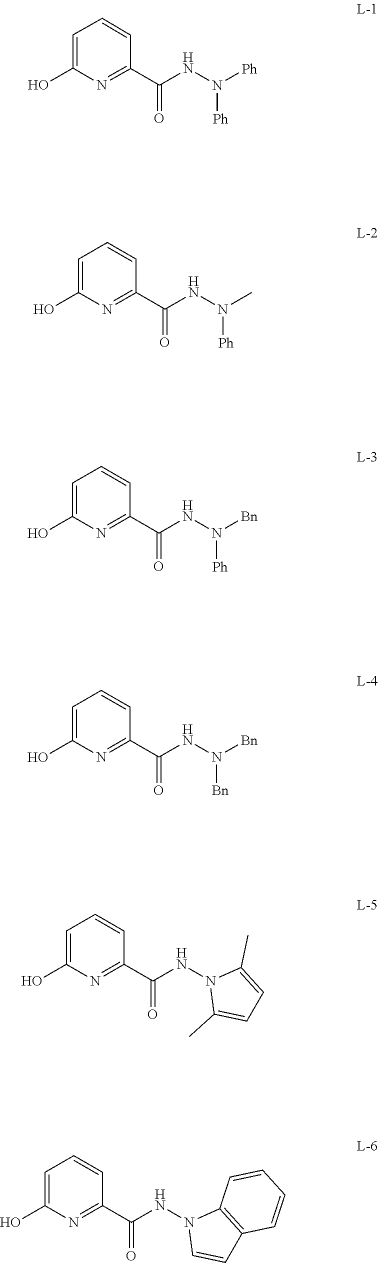 [SMILES: O=C(NN(c1ccccc1)c1ccccc1)c1cccc(O)n1 ; CN(NC(=O)c1cccc(O)n1)c1ccccc1 ; O=C(NN(Cc1ccccc1)c1ccccc1)c1cccc(O)n1 ; O=C(NN(Cc1ccccc1)Cc1ccccc1)c1cccc(O)n1 ; Cc1ccc(C)n1NC(=O)c1cccc(O)n1 ; O=C(Nn1ccc2ccccc21)c1cccc(O)n1]

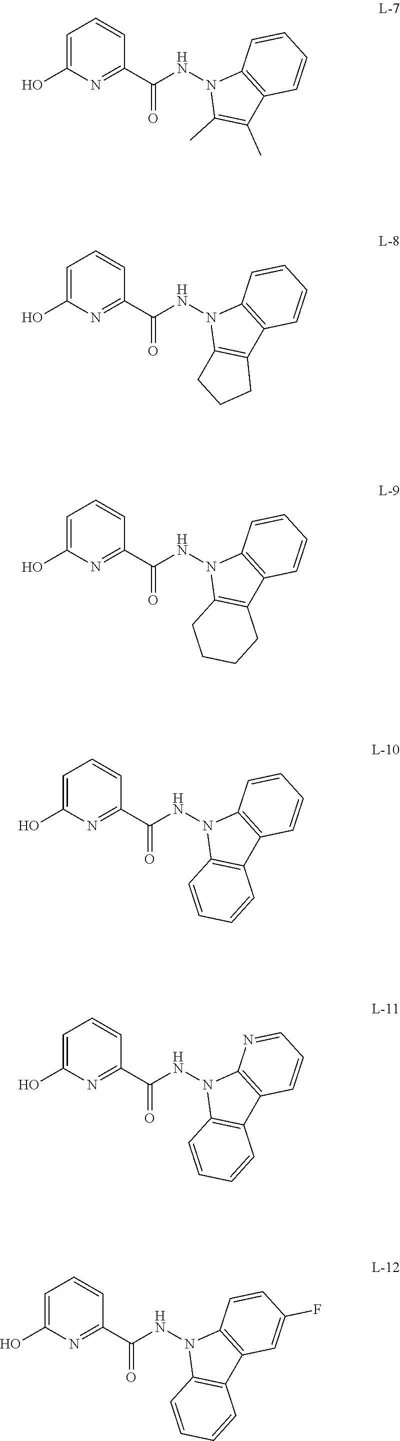 [SMILES: Cc1c(C)n(NC(=O)c2cccc(O)n2)c2ccccc12 ; O=C(Nn1c2c(c3ccccc31)CCC2)c1cccc(O)n1 ; O=C(Nn1c2c(c3ccccc31)CCCC2)c1cccc(O)n1 ; O=C(Nn1c2ccccc2c2ccccc21)c1cccc(O)n1 ; O=C(Nn1c2ccccc2c2cccnc21)c1cccc(O)n1 ; O=C(Nn1c2ccccc2c2cc(F)ccc21)c1cccc(O)n1]

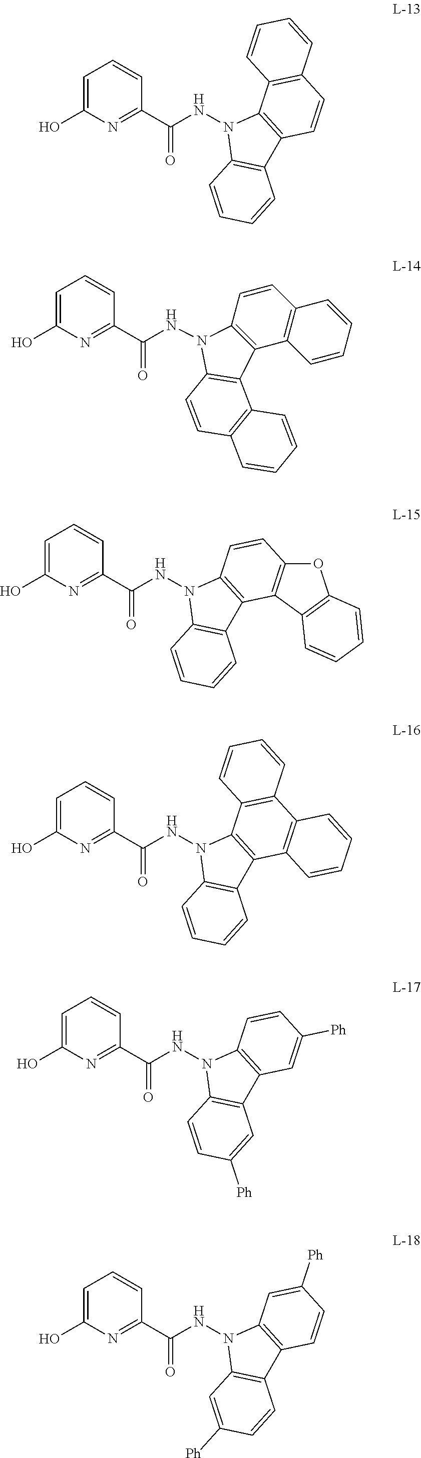 [SMILES: O=C(Nn1c2ccccc2c2ccc3ccccc3c21)c1cccc(O)n1 ; O=C(Nn1c2ccc3ccccc3c2c2c3ccccc3ccc21)c1cccc(O)n1 ; O=C(Nn1c2ccccc2c2c3c(ccc21)oc1ccccc13)c1cccc(O)n1 ; O=C(Nn1c2ccccc2c2c3ccccc3c3ccccc3c21)c1cccc(O)n1 ; O=C(Nn1c2ccc(-c3ccccc3)cc2c2cc(-c3ccccc3)ccc21)c1cccc(O)n1 ; O=C(Nn1c2cc(-c3ccccc3)ccc2c2ccc(-c3ccccc3)cc21)c1cccc(O)n1]

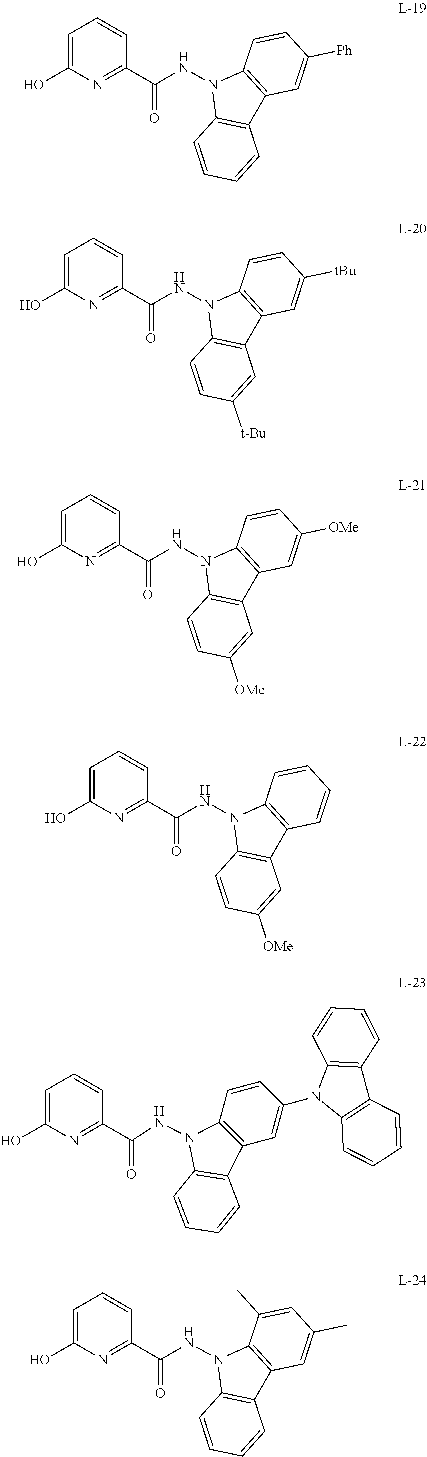 [SMILES: O=C(Nn1c2ccccc2c2cc(-c3ccccc3)ccc21)c1cccc(O)n1 ; CC(C)(C)c1ccc2c(c1)c1cc(C(C)(C)C)ccc1n2NC(=O)c1cccc(O)n1 ; COc1ccc2c(c1)c1cc(OC)ccc1n2NC(=O)c1cccc(O)n1 ; COc1ccc2c(c1)c1ccccc1n2NC(=O)c1cccc(O)n1 ; O=C(Nn1c2ccccc2c2cc(-n3c4ccccc4c4ccccc43)ccc21)c1cccc(O)n1 ; Cc1cc(C)c2c(c1)c1ccccc1n2NC(=O)c1cccc(O)n1]

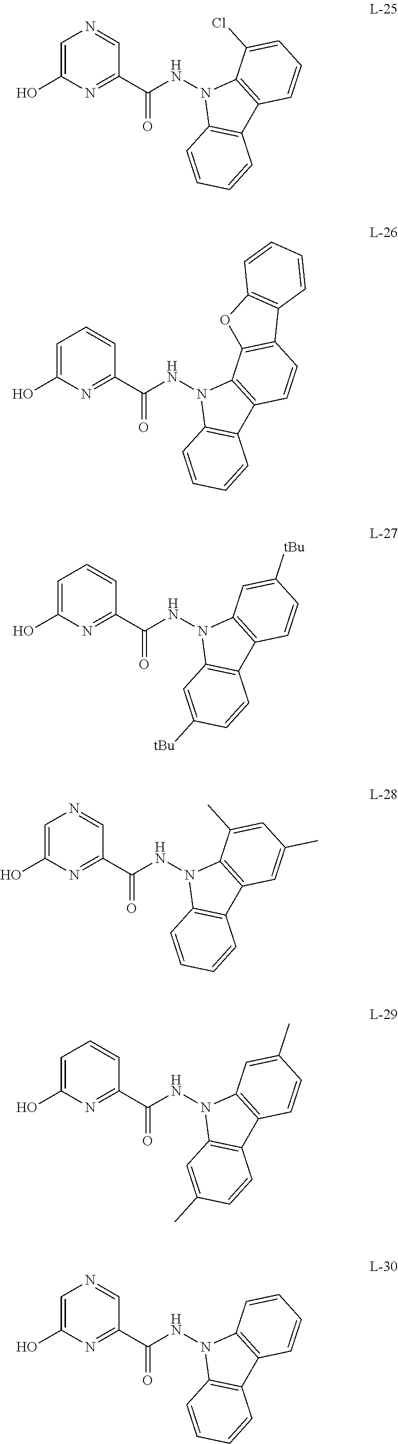 [SMILES: O=C(Nn1c2ccccc2c2cccc(Cl)c21)c1cncc(O)n1 ; O=C(Nn1c2ccccc2c2ccc3c4ccccc4oc3c21)c1cccc(O)n1 ; CC(C)(C)c1ccc2c3ccc(C(C)(C)C)cc3n(NC(=O)c3cccc(O)n3)c2c1 ; Cc1cc(C)c2c(c1)c1ccccc1n2NC(=O)c1cncc(O)n1 ; Cc1ccc2c3ccc(C)cc3n(NC(=O)c3cccc(O)n3)c2c1 ; O=C(Nn1c2ccccc2c2ccccc21)c1cncc(O)n1]

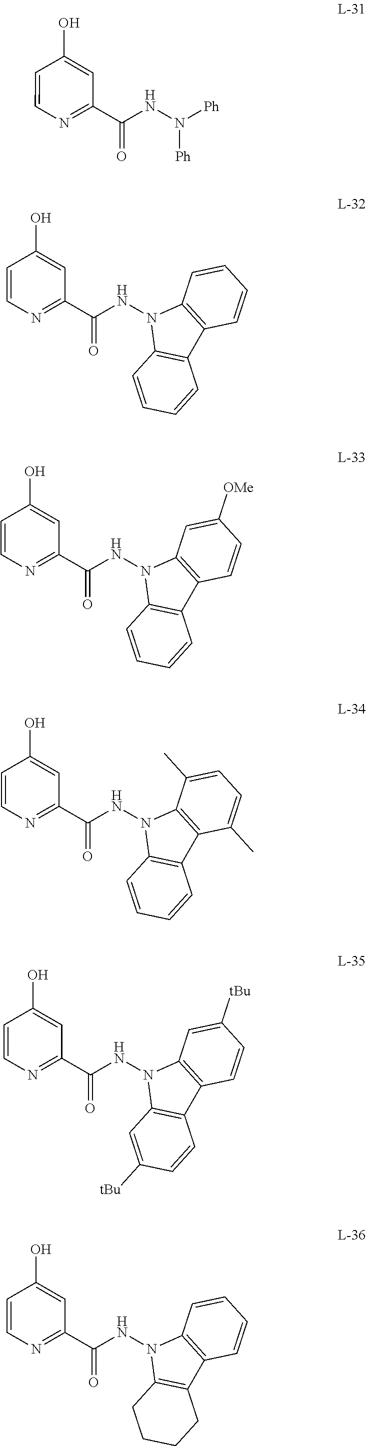 [SMILES: O=C(NN(c1ccccc1)c1ccccc1)c1cc(O)ccn1 ; O=C(Nn1c2ccccc2c2ccccc21)c1cc(O)ccn1 ; COc1ccc2c3ccccc3n(NC(=O)c3cc(O)ccn3)c2c1 ; Cc1ccc(C)c2c1c1ccccc1n2NC(=O)c1cc(O)ccn1 ; CC(C)(C)c1ccc2c3ccc(C(C)(C)C)cc3n(NC(=O)c3cc(O)ccn3)c2c1 ; O=C(Nn1c2c(c3ccccc31)CCCC2)c1cc(O)ccn1]

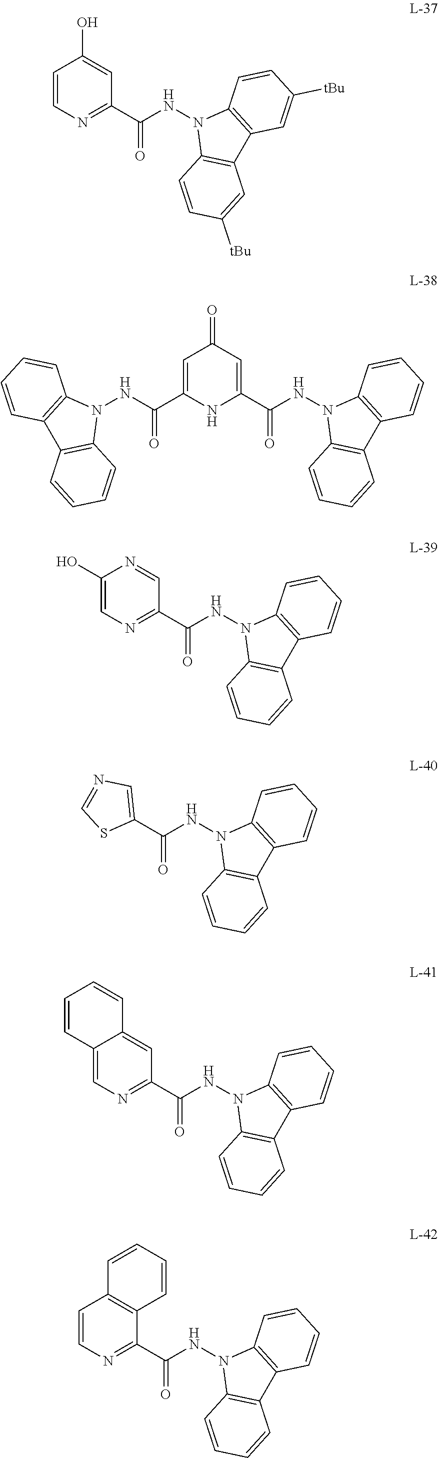 [SMILES: CC(C)(C)c1ccc2c(c1)c1cc(C(C)(C)C)ccc1n2NC(=O)c1cc(O)ccn1 ; O=C(Nn1c2ccccc2c2ccccc21)c1cc(=O)cc(C(=O)Nn2c3ccccc3c3ccccc32)[nH]1 ; O=C(Nn1c2ccccc2c2ccccc21)c1cnc(O)cn1 ; O=C(Nn1c2ccccc2c2ccccc21)c1cncs1 ; O=C(Nn1c2ccccc2c2ccccc21)c1cc2ccccc2cn1 ; O=C(Nn1c2ccccc2c2ccccc21)c1nccc2ccccc12]

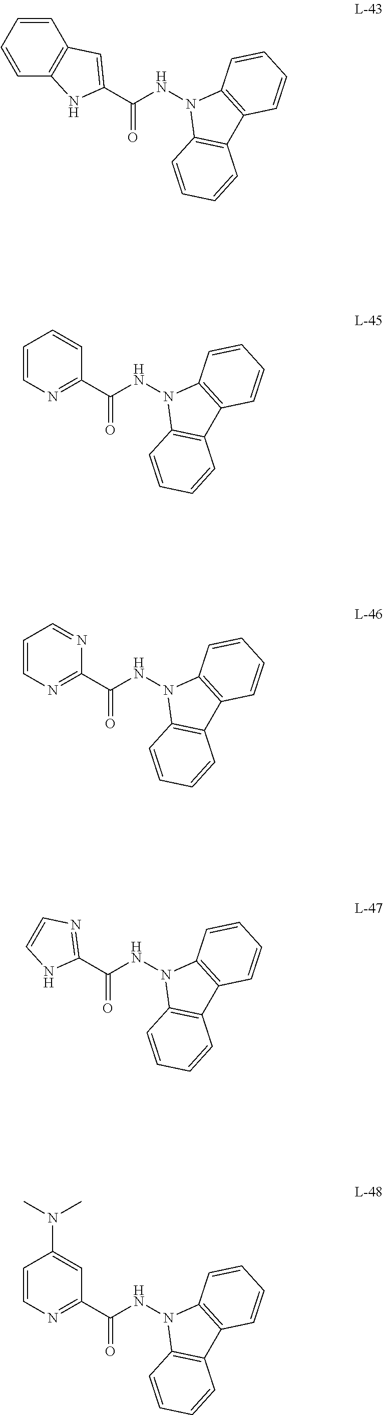 [SMILES: O=C(Nn1c2ccccc2c2ccccc21)c1cc2ccccc2[nH]1 ; O=C(Nn1c2ccccc2c2ccccc21)c1ccccn1 ; O=C(Nn1c2ccccc2c2ccccc21)c1ncccn1 ; O=C(Nn1c2ccccc2c2ccccc21)c1ncc[nH]1 ; CN(C)c1ccnc(C(=O)Nn2c3ccccc3c3ccccc32)c1]

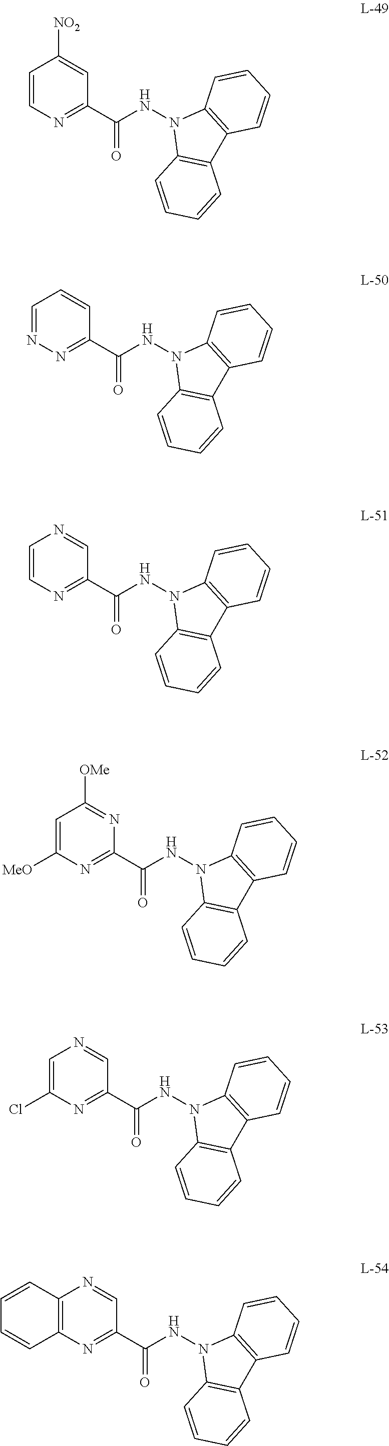 [SMILES: O=C(Nn1c2ccccc2c2ccccc21)c1cc([N+](=O)[O-])ccn1 ; O=C(Nn1c2ccccc2c2ccccc21)c1cccnn1 ; O=C(Nn1c2ccccc2c2ccccc21)c1cnccn1 ; COc1cc(OC)nc(C(=O)Nn2c3ccccc3c3ccccc32)n1 ; O=C(Nn1c2ccccc2c2ccccc21)c1cncc(Cl)n1 ; O=C(Nn1c2ccccc2c2ccccc21)c1cnc2ccccc2n1]

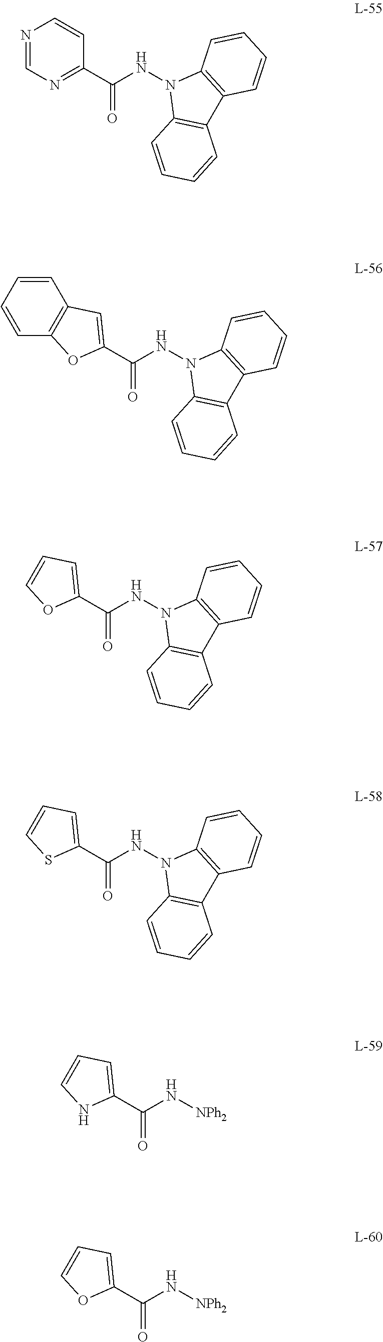

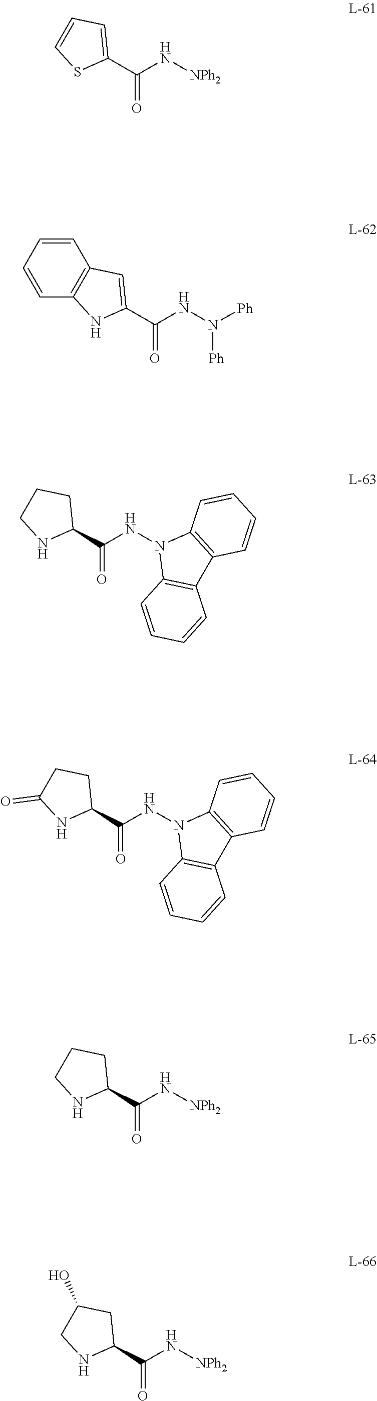

5. A method for coupling reaction of an aryl halide, characterized in that comprising: using a copper catalyst and using a compound as shown in formula I as a ligand, carrying out a coupling reaction of the aryl halide with a coupling reagent: 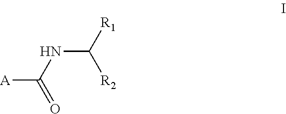
wherein, A, R₁, and R₂ are respectively as described in claim 1;
the aryl halide is selected from the group consisting of: aryl chloride, aryl bromide, or a combination thereof.

6. The method according to claim 5, characterized in that the reaction comprises: 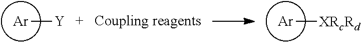
reacting 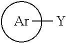
with a coupling reagent in an inert solvent to obtain a compound of 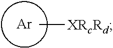
wherein, X is selected from the group consisting of: N, O, and S;
Y is selected from the group consisting of: Cl, Br, and I; 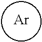
is selected from the group consisting of: substituted or unsubstituted C6-C20 aryl, substituted or unsubstituted 3- to 20-membered heteroaryl; wherein, the “substituted” refers to one or more hydrogen atoms in 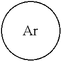
being substituted with substituents selected from the group consisting of: halogen, nitro, cyano, amino that is unsubstituted or substituted with one or two C1-C6 alkyl or C2-C10 acyl (alkyl-CO—), hydroxyl, unsubstituted or halogenated C1-C6 alkyl, C1-C6 alkoxy, C6-C10 aryl, 3- to 20-membered heteroaryl, C6-C10 aryl-O—, C2-C10 ester group (alkyl-COO—), C2-C10 acyl (alkyl-CO—), C2-C10 acyl-alkoxy (alkyl-OOC—), C2-C10 amido (alkyl-NHC(O)—, aryl-NHC(O)—), —COOH, hydroxy-C1-C10 alkylene, MeS—, sulfonyl and sulfonamide; wherein, two hydrogen atoms on two adjacent carbon atoms in 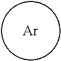
can be replaced by —(CH₂)ₙ— (n is 1, 2, 3, 4, 5 or 6);
the coupling reagent is selected from the group consisting of: ammonia water, ammonia gas, ammonium salt (preferably ammonium chloride, ammonium carbonate, ammonium sulfate, ammonium hydrogen phosphate, or a combination thereof)/hydroxide solution (preferably potassium hydroxide solution), 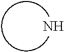
(the number of carbon atoms is 2 to 20), RₑC(O)NHR_{d}, R_{c}SO₂M (preferably, M is sodium or potassium), sodium azide, NHR_{c}R_{d}, R_{c}OH, R_{c}SH, hydroxides and salts that can hydrolyze to produce hydroxide, cyanides and cyanide salts that can ionize to form cyanide ions, aryl/heteroaryl terminal alkynes, and alkyl terminal alkynes;
R_{c}, R_{d}, and Rₑ are each independently selected from the group consisting of: none, H, substituted or unsubstituted C1-C6 alkyl, substituted or unsubstituted C1-C6 alkenyl, substituted or unsubstituted C6-C20 aryl, substituted or unsubstituted 3- to 20-membered heteroaryl, substituted or unsubstituted C7-C25 alkyl-aryl, substituted or unsubstituted C1-C5 alkyl-3- to 20-membered heteroaryl, substituted or unsubstituted C3-C20 cycloalkyl, substituted or unsubstituted C1-C5 alkyl-C3-C20 cycloalkyl, substituted or unsubstituted 3- to 20-membered heterocyclyl, or substituted or unsubstituted C1-C5 alkyl-3- to 20-membered heterocyclyl;
or R_{c} and R_{d} together form a substituted or unsubstituted C3-C20 cycloalkyl, a substituted or unsubstituted 3- to 20-membered heterocyclyl;
or Rₑ and R_{d} together form a substituted or unsubstituted C3-C20 cycloalkyl, a substituted or unsubstituted 3- to 20-membered heterocyclyl;
wherein, the heteroaryl or heterocyclyl contains 1 to 5 heteroatoms selected from the group consisting of: N, O, and S; the cycloalkyl or heterocyclyl may be a monocyclic, polycyclic, spirocyclic, or bridged ring structure;
the “substituted” refers to one or more hydrogen atoms in the group being substituted with substituents selected from the group consisting of: halogen, cyano, oxo (i.e. two hydrogen atoms on the same carbon atom on the group are replaced by ═O), C1-C6 alkyl, halogenated C1-C6 alkyl, C1-C6 alkoxy, C6-C10 aryl, C6-C10 aryl-O—, C2-C10 ester group (alkyl-COO—), C2-C10 acyl-alkoxy (alkyl-OOC—), C2-C10 acyl (alkyl-CO—), C2-C10 amido (alkyl/aryl-NHC(O)—), —COOH, nitro, hydroxyl, amino, amino that is substituted by one or two C1-C6 alkyl, and C1-C2 alkyl-S—.

7. The method according to claim 5, characterized in that in the reaction, the molar ratio of the ligand to the reactant aryl halide is (0.01 to 50): 100, preferably (0.01 to 5): 100; and/or
the molar ratio of the ligand to the copper catalyst is (1 to 5): 1, preferably (1 to 2): 1.

8. The method according to claim 5, characterized in that the reaction comprises the following (1), (2), (3) or (4):
(1) in an inert solvent, reacting 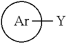
with NHR_{c}R_{d} to obtain 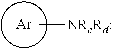 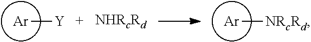
wherein, the definitions of each group are as described above;
(2) in an inert solvent, reacting 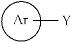
with H₂O to obtain 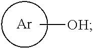 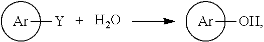
wherein, the definitions of each group are as described above;
(3) in an inert solvent, reacting 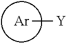
with R_{c}SO₂M to obtain 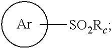 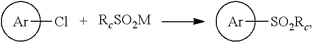
wherein, the definitions of each group are as described above.

9. A catalytic system for aryl coupling reaction, characterized in that comprising: copper catalyst, ligand, base, and inert solvent;
wherein, the copper catalyst is selected from the group consisting of: Cu, CuI, CuBr, CuCl, CuTc, Cu(OAc)₂, CuSO₄, Cu₂O, CuBr₂, CuCl₂, CuO, CuSCN, CuCN, Cu(OTf)₂, Cu(acac)₂, or a combination thereof;
the base is selected from the group consisting of: potassium carbonate, cesium carbonate, potassium phosphate, sodium bicarbonate, potassium bicarbonate, sodium carbonate, sodium hydroxide, potassium hydroxide, lithium hydroxide, sodium acetate, sodium methoxide, sodium ethoxide, sodium tert-butoxide, potassium ter-butoxide, or combinations thereof;
the inert solvent is selected from the group consisting of: DMSO, DMF, DMA, NMP, acetonitrile, ethanol, ethylene glycol, tert-butanol, polyethylene glycol-200, isopropanol, 2-methoxyethanol, toluene, methyl isobutyl ketone, propylene carbonate, water, sulfolane, 1-butanol, acetone, 1,4-dioxane, tetrahydrofuran, DME, methanol, toluene, or combinations thereof;
the ligand has a structure shown in formula (I): 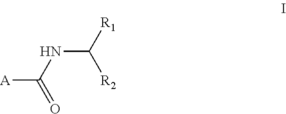
wherein, A, R₁, and R₂ are respectively as described in claim 1.

10. A compound as shown in formula I; 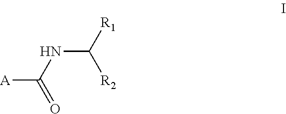
wherein, A is a saturated or unsaturated C5-C6 carbocycle, or a 5- or 6-membered carbon heterocarbocycle, or a benzo saturated or unsaturated 5- or 6-membered carbon heterocarbocycle, wherein the carbon heterocarbocycle has 1 to 5 heteroatoms selected from the group consisting of: N, O, and S; A is optionally substituted by substituents selected from the group consisting of: hydroxyl, nitro, —NRaRb, halogen, C1-4 alkyl, C1-4 alkoxy, phenyl, and oxo (i.e., two hydrogen atoms on the same carbon atom in the group are replaced by ═O);
Ra and Rb are each independently selected from the group consisting of: H, hydroxyl, amino, halogen, and C1-4 alkyl;
R₁ and R₂ are each independently selected from the group consisting of: H, substituted or unsubstituted C1-C6 alkyl, substituted or unsubstituted C1-C6 alkoxy, substituted or unsubstituted C3-C6 cycloalkyl, substituted or unsubstituted 3- to 6-membered heterocyclyl, substituted or unsubstituted C6-C20 aryl, substituted or unsubstituted 5- to 20-membered heteroaryl, substituted or unsubstituted benzyl; or, R₁ and R₂, together with the N atom to which they are attached, form a 3- to 20-membered saturated or unsaturated carbocycle or heterocycle;
unless otherwise specified, the “substituted” refers to one or more hydrogen atoms in the group being substituted with substituents selected from the group consisting of: C1-C6 alkyl, C1-C6 alkoxy, C6-C10 aryl, 3- to 20-membered heteroaryl, C6-C10 aryl-O—, halogen, nitro, hydroxyl, cyano, C2-C10 ester group (alkyl-COO—), C2-C10 acyl (alkyl-CO—), sulfonate, or two hydrogen atoms on two adjacent carbon atoms in the group are replaced by —(CH₂)ₙ— (n is 1, 2, 3, 4, 5 or 6).

11. The compound according to claim 10, characterized in that the compound of formula I is selected from the group consisting of: 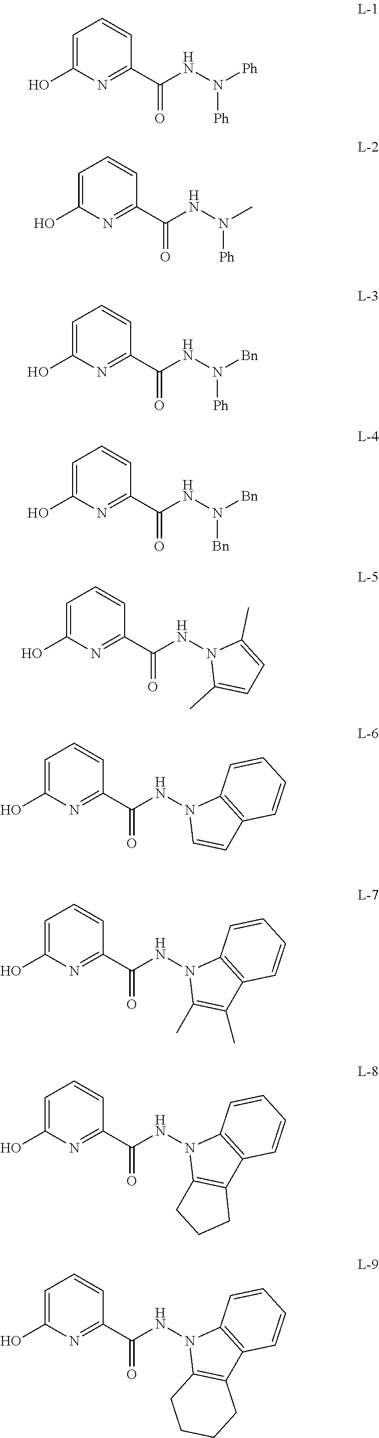 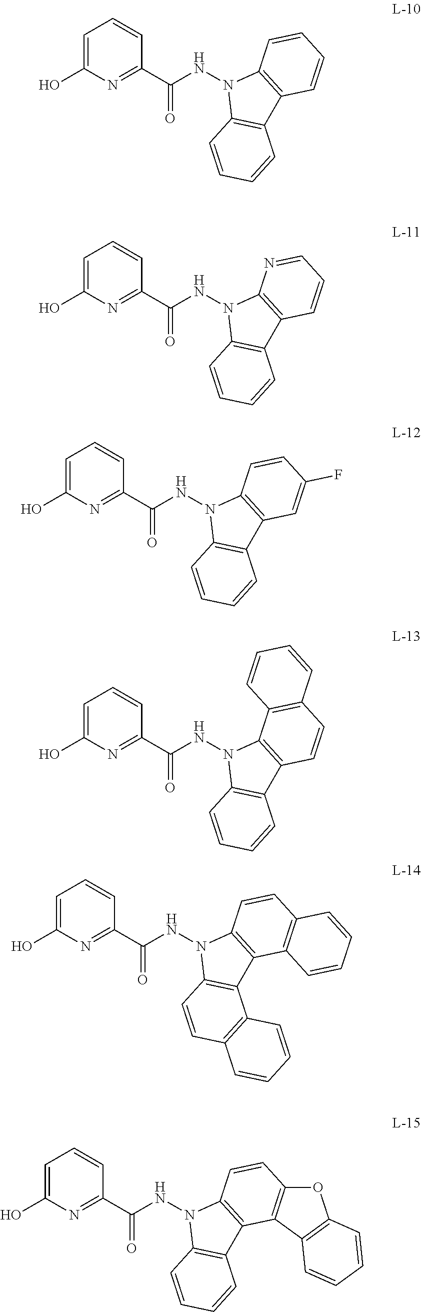 [SMILES: O=C(Nn1c2ccccc2c2ccccc21)c1cccc(O)n1 ; O=C(Nn1c2ccccc2c2cccnc21)c1cccc(O)n1 ; O=C(Nn1c2ccccc2c2cc(F)ccc21)c1cccc(O)n1 ; O=C(Nn1c2ccccc2c2ccc3ccccc3c21)c1cccc(O)n1 ; O=C(Nn1c2ccc3ccccc3c2c2c3ccccc3ccc21)c1cccc(O)n1 ; O=C(Nn1c2ccccc2c2c3c(ccc21)oc1ccccc13)c1cccc(O)n1] 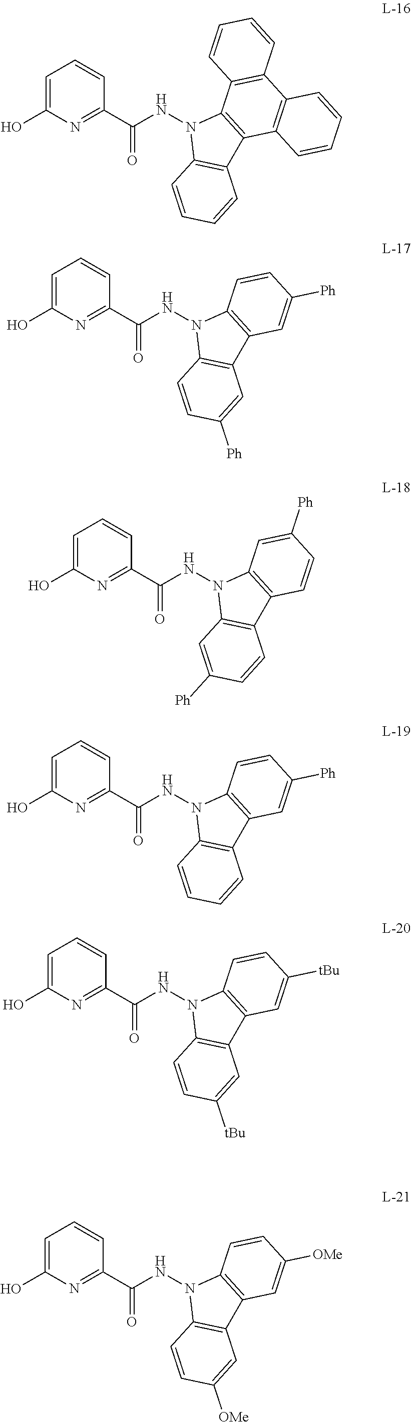 [SMILES: O=C(Nn1c2ccccc2c2c3ccccc3c3ccccc3c21)c1cccc(O)n1 ; O=C(Nn1c2ccc(-c3ccccc3)cc2c2cc(-c3ccccc3)ccc21)c1cccc(O)n1 ; O=C(Nn1c2cc(-c3ccccc3)ccc2c2ccc(-c3ccccc3)cc21)c1cccc(O)n1 ; O=C(Nn1c2ccccc2c2cc(-c3ccccc3)ccc21)c1cccc(O)n1 ; CC(C)(C)c1ccc2c(c1)c1cc(C(C)(C)C)ccc1n2NC(=O)c1cccc(O)n1 ; COc1ccc2c(c1)c1cc(OC)ccc1n2NC(=O)c1cccc(O)n1] 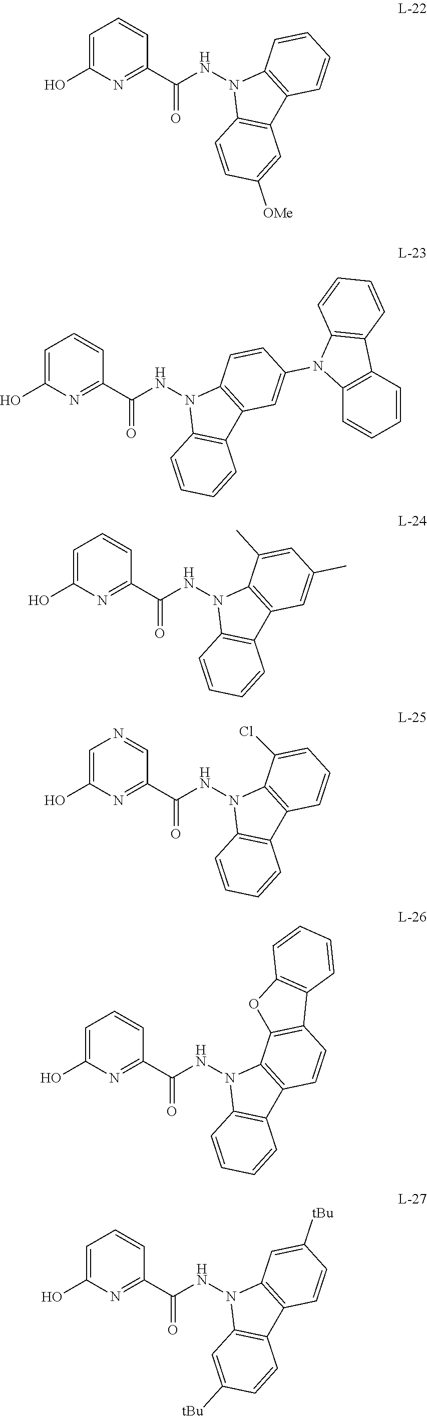 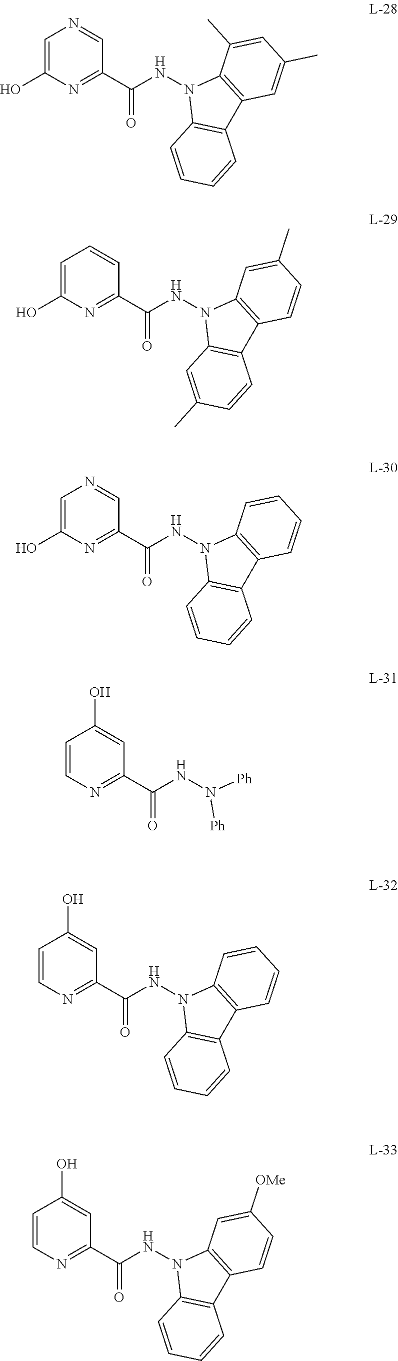 [SMILES: Cc1cc(C)c2c(c1)c1ccccc1n2NC(=O)c1cncc(O)n1 ; Cc1ccc2c3ccc(C)cc3n(NC(=O)c3cccc(O)n3)c2c1 ; O=C(Nn1c2ccccc2c2ccccc21)c1cncc(O)n1 ; O=C(NN(c1ccccc1)c1ccccc1)c1cc(O)ccn1 ; O=C(Nn1c2ccccc2c2ccccc21)c1cc(O)ccn1 ; COc1ccc2c3ccccc3n(NC(=O)c3cc(O)ccn3)c2c1] 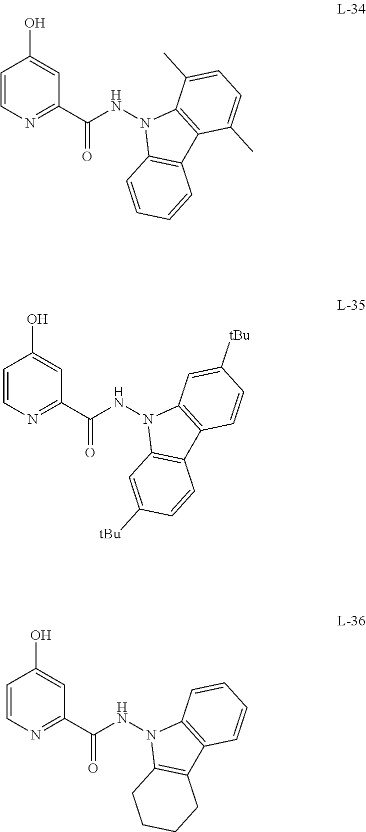 [SMILES: Cc1ccc(C)c2c1c1ccccc1n2NC(=O)c1cc(O)ccn1 ; CC(C)(C)c1ccc2c3ccc(C(C)(C)C)cc3n(NC(=O)c3cc(O)ccn3)c2c1 ; O=C(Nn1c2c(c3ccccc31)CCCC2)c1cc(O)ccn1] 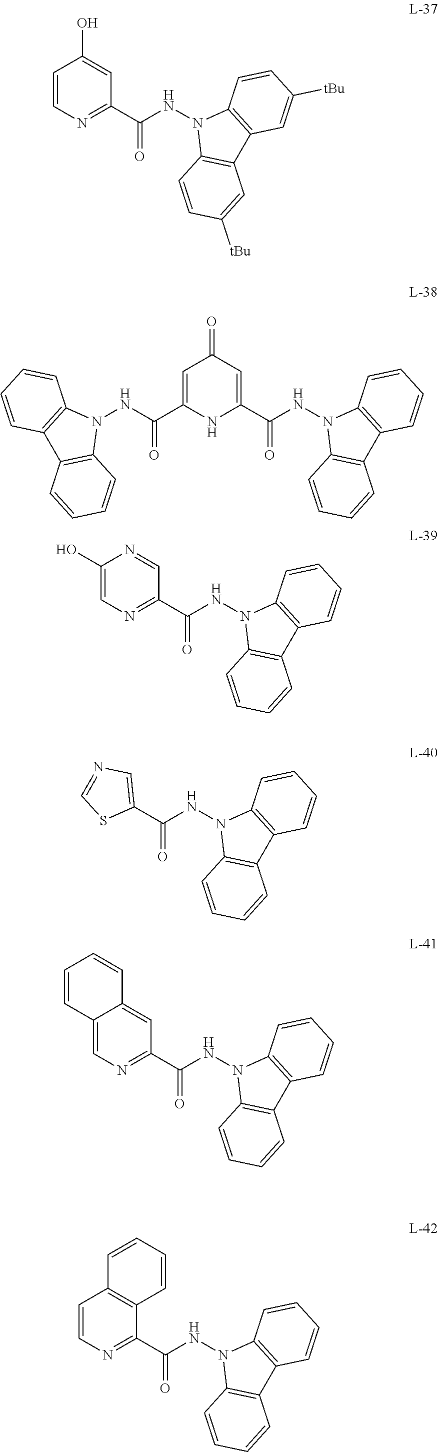 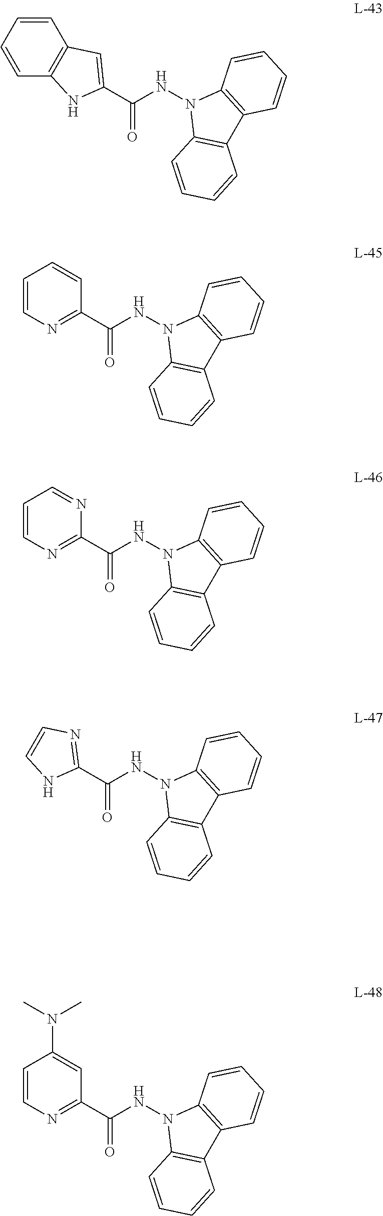 [SMILES: O=C(Nn1c2ccccc2c2ccccc21)c1cc2ccccc2[nH]1 ; O=C(Nn1c2ccccc2c2ccccc21)c1ccccn1 ; O=C(Nn1c2ccccc2c2ccccc21)c1ncccn1 ; O=C(Nn1c2ccccc2c2ccccc21)c1ncc[nH]1 ; CN(C)c1ccnc(C(=O)Nn2c3ccccc3c3ccccc32)c1] 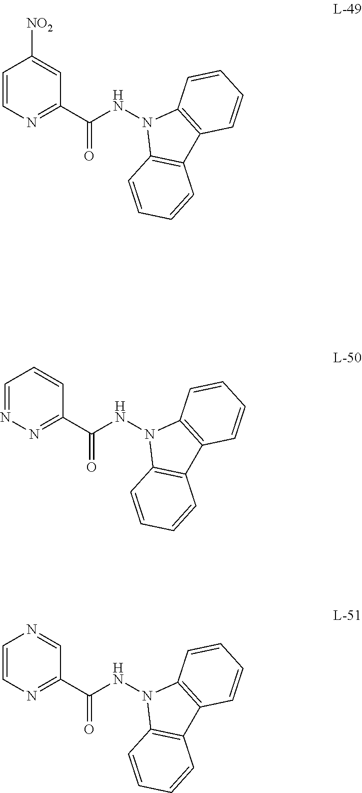 [SMILES: O=C(Nn1c2ccccc2c2ccccc21)c1cc([N+](=O)[O-])ccn1 ; O=C(Nn1c2ccccc2c2ccccc21)c1cccnn1 ; O=C(Nn1c2ccccc2c2ccccc21)c1cnccn1] 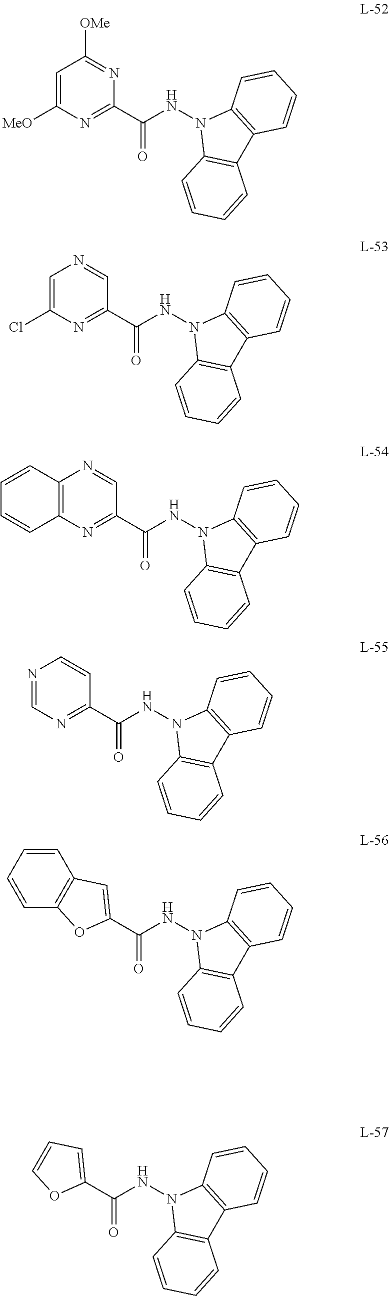 [SMILES: COc1cc(OC)nc(C(=O)Nn2c3ccccc3c3ccccc32)n1 ; O=C(Nn1c2ccccc2c2ccccc21)c1cncc(Cl)n1 ; O=C(Nn1c2ccccc2c2ccccc21)c1cnc2ccccc2n1 ; O=C(Nn1c2ccccc2c2ccccc21)c1ccncn1 ; O=C(Nn1c2ccccc2c2ccccc21)c1cc2ccccc2o1 ; O=C(Nn1c2ccccc2c2ccccc21)c1ccco1] 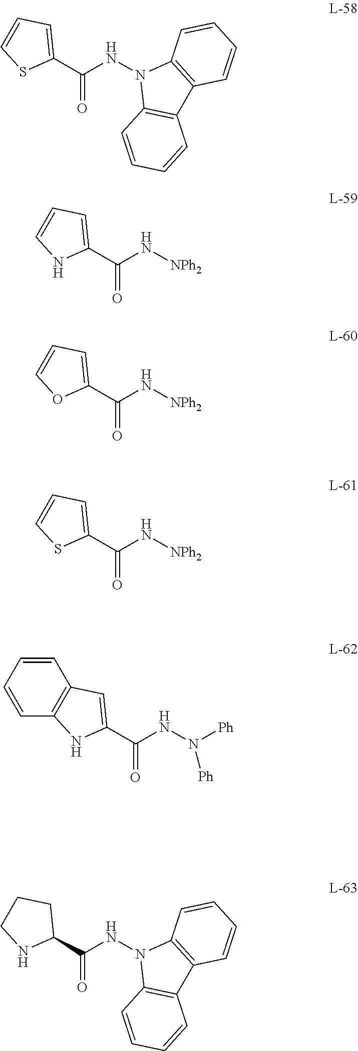 [chemical structure]
